(19) United States
(12) Patent Application Publication
(10) Pub. No.: US 2024/0315129 A1
(43) Pub. Date: Sep. 19, 2024

(54) Diarylamine Compound and Use Thereof, Light Extraction Material, Electroluminescent Device, and Display Apparatus

(71) Applicant: BOE Technology Group Co., Ltd., Beijing (CN)

(72) Inventors: Lei CHEN, Beijing (CN); Dan WANG, Beijing (CN); Bingyan LIANG, Beijing (CN); Xueqin CHEN, Beijing (CN); Dongxu ZHANG, Beijing (CN)

(21) Appl. No.: 18/273,262

(22) PCT Filed: Dec. 22, 2021
(86) PCT No.: PCT/CN2021/140537

§ 371 Date: Jul. 19, 2023

(51) Int. Cl. H10K 85/60; C07D 235/18; C07D 263/57; C07D 277/66; C07D 409/14; C07D 413/12; C07D 413/14; C07D 417/14; C09K 11/06; H10K 50/858

(52) U.S. Cl. CPC H10K 85/636; C07D 235/18; C07D 263/57; C07D 277/66; C07D 409/14; C07D 413/12; C07D 413/14; C07D 417/14; C09K 11/06; H10K 85/633; C09K 2211/1018; H10K 50/858; H10K 85/615; H10K 85/626; H10K 85/654; H10K 85/657; H10K 85/6572; H10K 85/6574; H10K 85/6576

(57) ABSTRACT

A structural general formula of a diarylamine compound is formula (I):

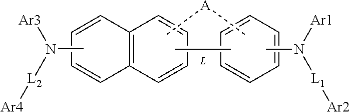

### CROSS-REFERENCE TO RELATED APPLICATION

This application is a national stage application of PCT Application No. PCT/CN2021/140537, which is filed on Dec. 22, 2021, and entitled “Diarylamine Compound and Use Thereof, Light Extraction Material, Electroluminescent Device, and Display Device”, the content of which should be regarded as being incorporated herein by reference.

### TECHNICAL FIELD

Embodiments of the present disclosure relate to, but are not limited to, the field of display technology, in particular to a diarylamine compound and use thereof, a light extraction material, an electroluminescent device and a display apparatus.

### BACKGROUND

In recent years, as a new type of flat panel display, Organic Light Emitting Devices (OLEDs) have gradually attracted more and more attention. An OLED device is composed of a light emitting layer and a pair of electrodes on both sides of the light emitting layer. When an electric field is applied between the two electrodes, electrons are injected from a negative electrode and holes are injected from a positive electrode. In the light emitting layer, the electrons and the holes recombine to form an excited state, and energy generated when the excited state returns to a ground state will emit light. OLED devices have become hot mainstream display products on the market at present because of their characteristics such as active luminescence, high luminous brightness and efficiency, high resolution, wide color gamut and viewing angle, high response speed, low energy consumption and flexibility.

In recent years, the application field of OLED devices has been extended from mobile phones to other high-quality information display apparatuses. With the continuous development of product types and the requirements of various display apparatuses, the demand for OLED devices is increasing, and there is a necessity to develop devices with higher resolution, higher efficiency, lower voltage and longer service life. Improvement and optimization of performance of OLED devices may be achieved by improving different functional layers and combinations thereof in the devices.

When light is transmitted between different media, a loss will occur at a contact surface of the media due to a difference in refractive index. A material of a light extraction layer (also referred to as a capping layer (CPL)) may effectively improve a light output efficiency of an OLED device. The light extraction layer may be a layer of an organic or inorganic transparent material with a high refractive index in the OLED device, with a low absorption intensity in a visible light range, which is close to zero. By introducing a light extraction layer into an OLED device, an external quantum efficiency of the device may be obviously improved, and a light loss inside the device may be reduced, thus improving the efficiency of the device. Moreover, the light extraction layer may absorb UV light, thereby avoiding an influence of UV light on the stability of the device.

### SUMMARY

The following is a summary of subject matters described herein in detail. The summary is not intended to limit the protection scope of the present application.

An embodiment of the present disclosure provides a diarylamine compound, having a general structural formula:

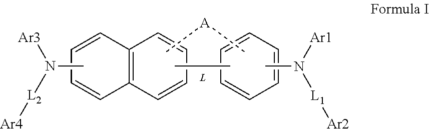

wherein A is not present or present;
i) when A is not present, a naphthalene ring is linked with a benzene ring by a bond L;
at least two of Ar₁ to Ar₄ include groups represented by General Formula II and/or General Formula III:

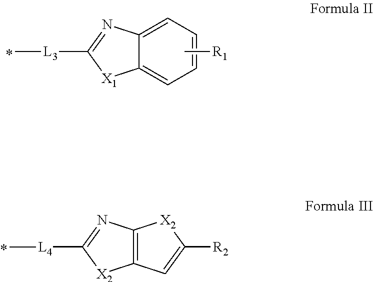

the remaining of Ar₁ to Ar₄ that do not include groups represented by General Formula II or General Formula III include any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
X₁ includes CR₃R₄, O, NR₅ or S; X₂ includes O or S;
R₁ to R₅ each independently include any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 to C50 heteroaryl, and when R₁ and R₂ each independently include substituted or unsubstituted C6 to C50 aryl and substituted or unsubstituted C2 to C50 heteroaryl, R₁ or R₂ and a benzene ring in General Formula II or III are respectively linked by a single bond or fused by sharing two atoms; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ to L₄ each independently include any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene;
ii) when A is present, A forms a five-membered ring with the bond L, and A includes any one of O, S, NR₆ and CR₇R₈;
at least one of Ar₁ to Ar₄ includes a group represented by General Formula IV or V:

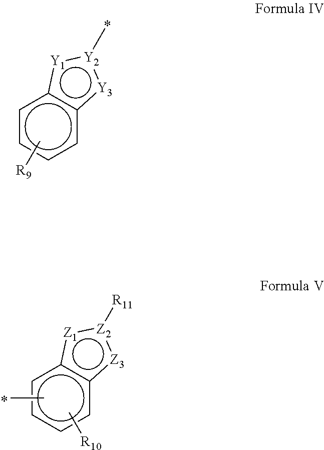

and at least one of Ar₁ to Ar₄ includes a group represented by General Formula VI:

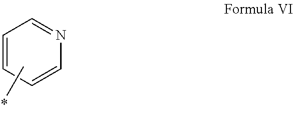

Y₁ and Z₁ each independently include any one of CR₁₂R₁₃, O, NR₁₄ and S, Y₂ and Z₂ each independently include C or N, and Y₃ and Z₃ each include N;
the remaining of Ar₁ to Ar₄ that do not include groups represented by General Formulas IV, V and VI include any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
R₆ to R₁₄ each independently include any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 to C50 heteroaryl; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ and L₂ each independently include any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene.

An embodiment of the present disclosure further provides use of the diarylamine compound described above as a light extraction material.

An embodiment of the present disclosure further provides a light extraction material, including the diarylamine compound described above.

An embodiment of the present disclosure further provides an electroluminescent device, including the diarylamine compound described above.

An embodiment of the present disclosure further provides a display apparatus, including the electroluminescent device described above.

Other aspects may be understood upon reading and understanding the drawings and detailed description.

### BRIEF DESCRIPTION OF DRAWINGS

Accompanying drawings are used for providing understanding of technical solutions of the present disclosure, and form a part of the specification. They are used for explaining the technical solutions of the present disclosure together with the embodiments of the present disclosure, but do not form a limitation on the technical solutions of the present disclosure.

FIG. 1 is a schematic diagram of structure of an electroluminescent device according to an exemplary embodiment of the present disclosure.

Meanings of reference signs in the accompanying drawings are as follows:
100—anode; 200—hole injection layer; 300—hole transport layer; 400—electron block layer; 500—light emitting layer; 600—hole block layer; 700—electron transport layer; 800—electron injection layer; 900—cathode; and 1000—light extraction layer.

### DETAILED DESCRIPTION

Implementations herein may be implemented in multiple different forms. Those of ordinary skills in the art may readily appreciate a fact that the implementations and contents may be varied into various forms without departing from the spirit and scope of the present disclosure. Therefore, the present disclosure should not be explained as being limited to contents described in following implementations only. The embodiments in the present disclosure and features in the embodiments may be combined randomly with each other without conflict.

In the accompanying drawings, a size of a constituent element, and a thickness of a layer or a region may be sometimes exaggerated for clarity. Therefore, any one implementation of the present disclosure is not necessarily limited to dimensions shown in the drawings, and the shapes and sizes of the components in the accompanying drawings do not reflect actual scales. In addition, the accompanying drawings schematically show an ideal example, and any one implementation of the present disclosure is not limited to the shapes, numerical values, or the like shown in the accompanying drawings.

An embodiment of the present disclosure provides a diarylamine compound, having a general structural formula:

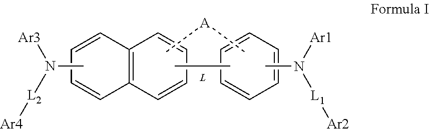

wherein A is not present or present;
i) when A is not present, a naphthalene ring is linked with a benzene ring by a bond L;
at least two of Ar₁ to Ar₄ include groups represented by General Formula II and/or General Formula III:

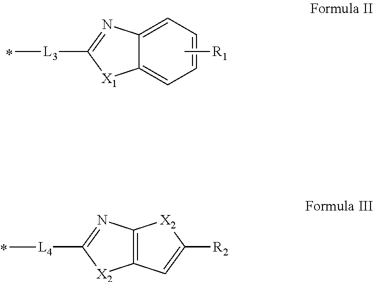

the remaining of Ar₁ to Ar₄ that do not include groups represented by General Formula II or General Formula III include any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
X₁ includes CR₃R₄, O, NR₅ or S; X₂ includes O or S;
R₁ to R₅ each independently include any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 to C50 heteroaryl, and when R₁ and R₂ each independently include substituted or unsubstituted C6 to C50 aryl and substituted or unsubstituted C2 to C50 heteroaryl, R₁ or R₂ and a benzene ring in General Formula II or III are respectively linked by a single bond or fused by sharing two atoms; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ to L₄ each independently include any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene;
ii) when A is present, A forms a five-membered ring with the bond L, and A includes any one of O, S, NR₆ and CR₇R₈;
at least one of Ar₁ to Ar₄ includes a group represented by General Formula IV or V:

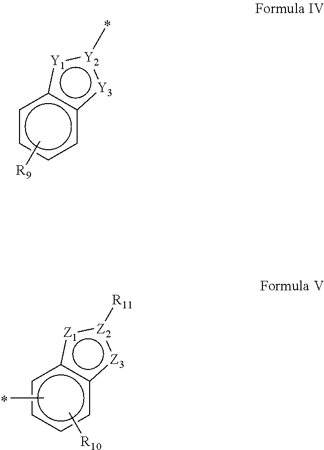

and at least one of Ar₁ to Ar₄ includes a group represented by General Formula VI:

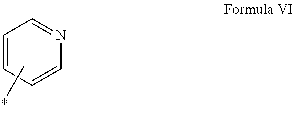

Y₁ and Z₁ each independently include any one of CR₁₂R₁₃, O, NR₁₄ and S, Y₂ and Z₂ each independently include C or N, and Y₃ and Z₃ each include N;
the remaining of Ar₁ to Ar₄ that do not include groups represented by General Formulas IV, V and VI include any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
R₆ to R₁₄ each independently include any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 to C50 heteroaryl; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ and L₂ each independently include any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene.

In an exemplary embodiment, the group represented by General Formula II may include any one of the following groups:

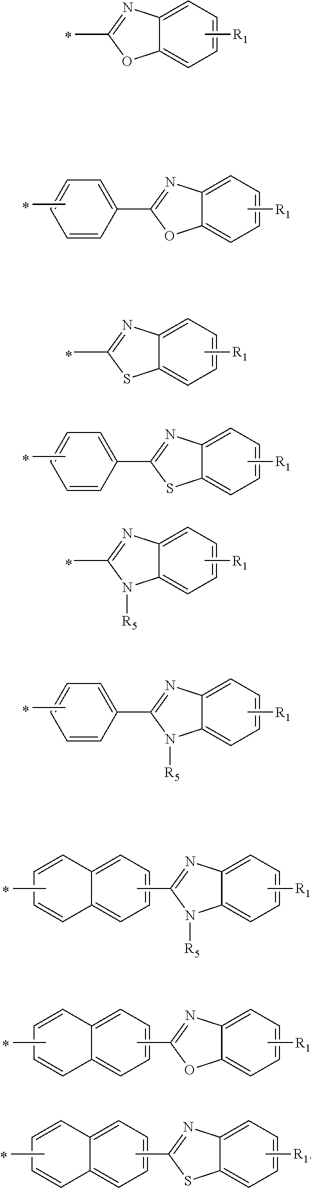

In an exemplary embodiment, the group represented by General Formula II may include any one of the following groups:

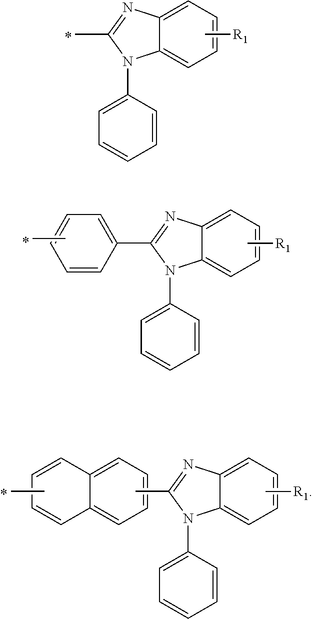

In an exemplary embodiment, the group represented by General Formula IV may include:

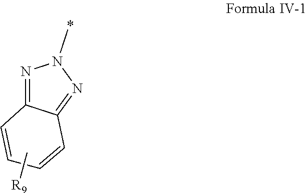

at least one of Ar₁ to Ar₄ includes a group represented by General Formula IV-1.

In an exemplary embodiment, the group represented by General Formula V may include:

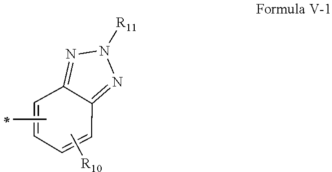

at least one of Ar₁ to Ar₄ includes a group represented by General Formula V-1.

In the groups represented by General Formula IV-1 and General Formula V-1, a five-membered ring contains three N atoms. The presence of multiple N atoms increases a density of electron clouds between lone pair electrons, increasing conjugation. Moreover, the N atom forms a hydrogen bond with H on an adjacent benzene ring, which increases planarity of two aromatic rings and increases polarizability of fragments, and compared with heterocycles with N substitution at other positions, the groups represented by General Formula IV-1 and General Formula V-1 have better conjugation and planarity.

In an exemplary embodiment, the group represented by General Formula IV may include:

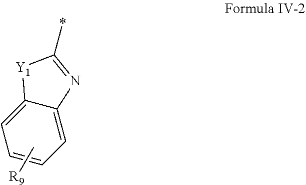

The group represented by General Formula V may include:

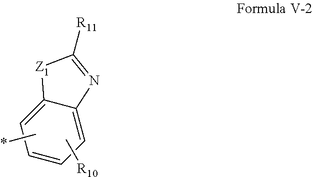

at least one of Ar₁ to Ar₄ includes a group represented by General Formula IV-2 or V-2.

In an exemplary embodiment, the group represented by General Formula IV-2 may include:

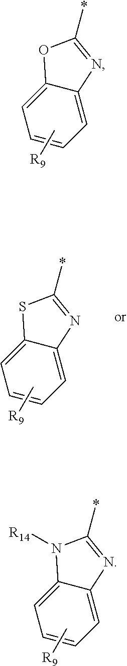

In an exemplary embodiment, the group represented by General Formula V-2 may include:

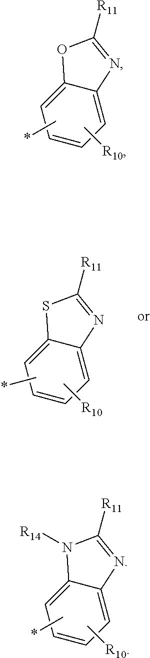

In an exemplary embodiment, the diarylamine compound may include any one of the following compounds:

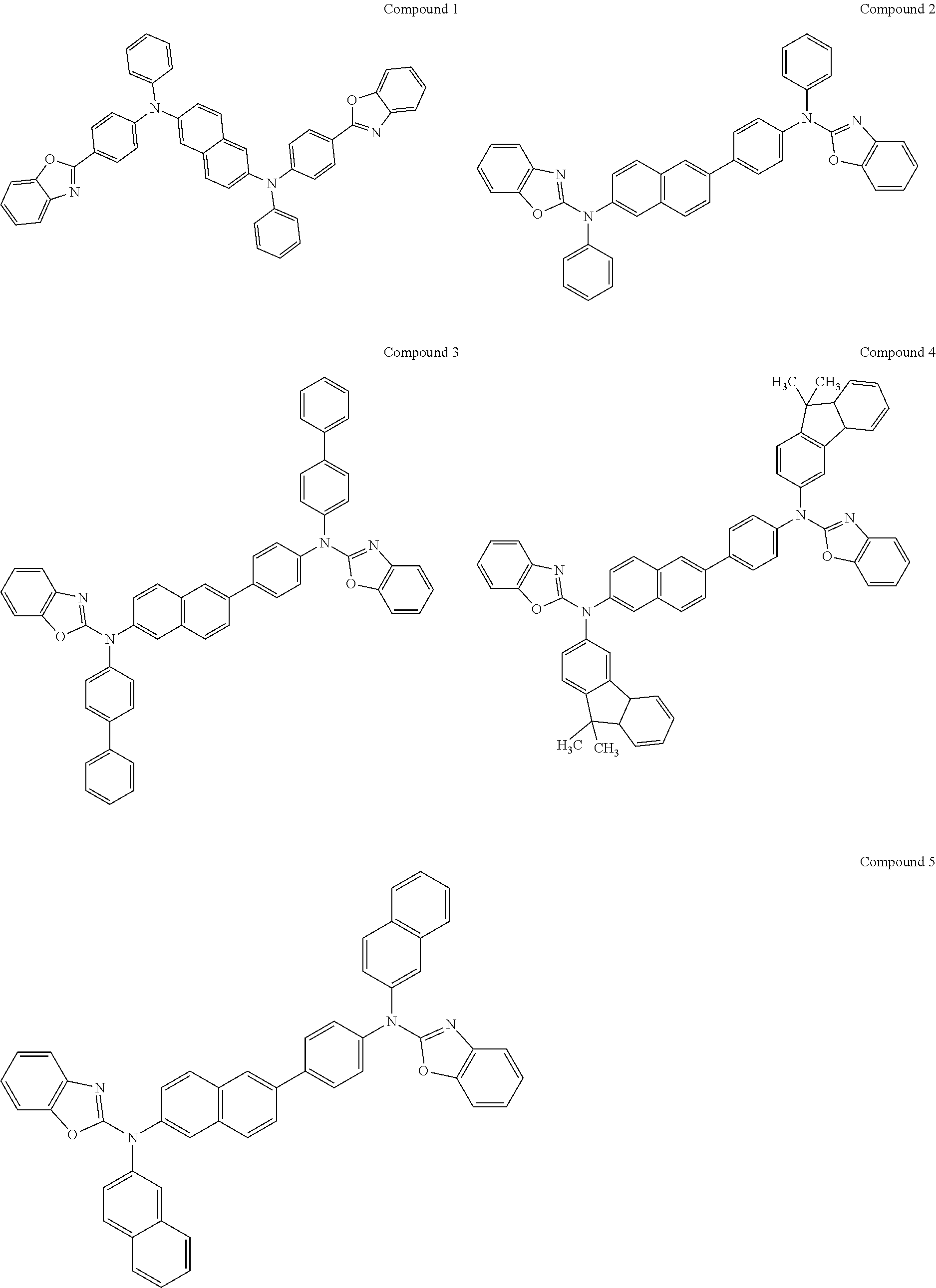 [SMILES: c1ccc(N(c2ccc(-c3nc4ccccc4o3)cc2)c2ccc3cc(N(c4ccccc4)c4ccc(-c5nc6ccccc6o5)cc4)ccc3c2)cc1 ; c1ccc(N(c2ccc(-c3ccc4cc(N(c5ccccc5)c5nc6ccccc6o5)ccc4c3)cc2)c2nc3ccccc3o2)cc1 ; c1ccc(-c2ccc(N(c3ccc(-c4ccc5cc(N(c6ccc(-c7ccccc7)cc6)c6nc7ccccc7o6)ccc5c4)cc3)c3nc4ccccc4o3)cc2)cc1 ; CC1(C)c2ccc(N(c3ccc(-c4ccc5cc(N(c6ccc7c(c6)C6C=CC=CC6C7(C)C)c6nc7ccccc7o6)ccc5c4)cc3)c3nc4ccccc4o3)cc2C2C=CC=CC21 ; c1ccc2cc(N(c3ccc(-c4ccc5cc(N(c6ccc7ccccc7c6)c6nc7ccccc7o6)ccc5c4)cc3)c3nc4ccccc4o3)ccc2c1]

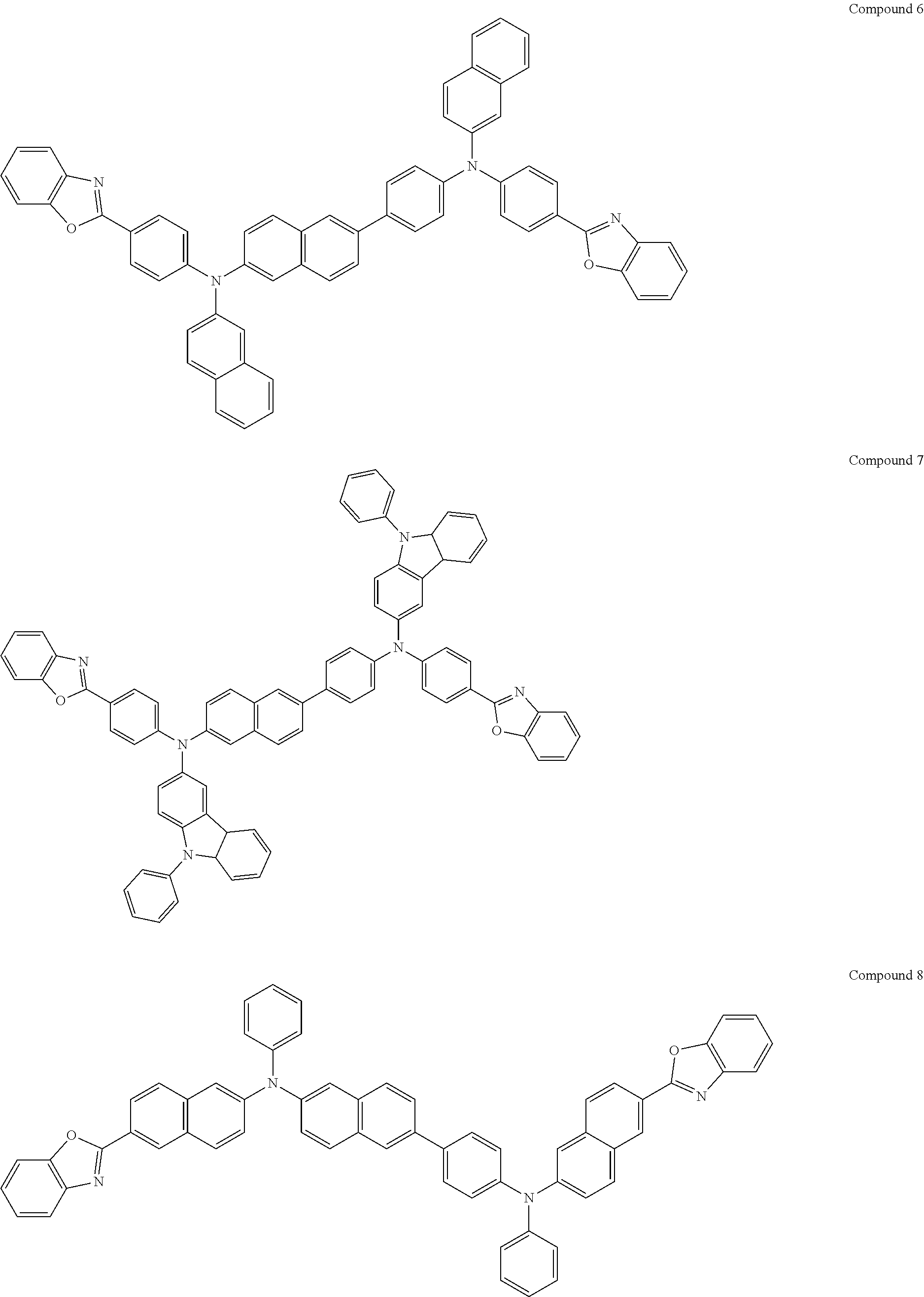 [SMILES: c1ccc2cc(N(c3ccc(-c4ccc5cc(N(c6ccc(-c7nc8ccccc8o7)cc6)c6ccc7ccccc7c6)ccc5c4)cc3)c3ccc(-c4nc5ccccc5o4)cc3)ccc2c1 ; C1=CC2c3cc(N(c4ccc(-c5ccc6cc(N(c7ccc(-c8nc9ccccc9o8)cc7)c7ccc8c(c7)C7C=CC=CC7N8c7ccccc7)ccc6c5)cc4)c4ccc(-c5nc6ccccc6o5)cc4)ccc3N(c3ccccc3)C2C=C1 ; c1ccc(N(c2ccc(-c3ccc4cc(N(c5ccccc5)c5ccc6cc(-c7nc8ccccc8o7)ccc6c5)ccc4c3)cc2)c2ccc3cc(-c4nc5ccccc5o4)ccc3c2)cc1]

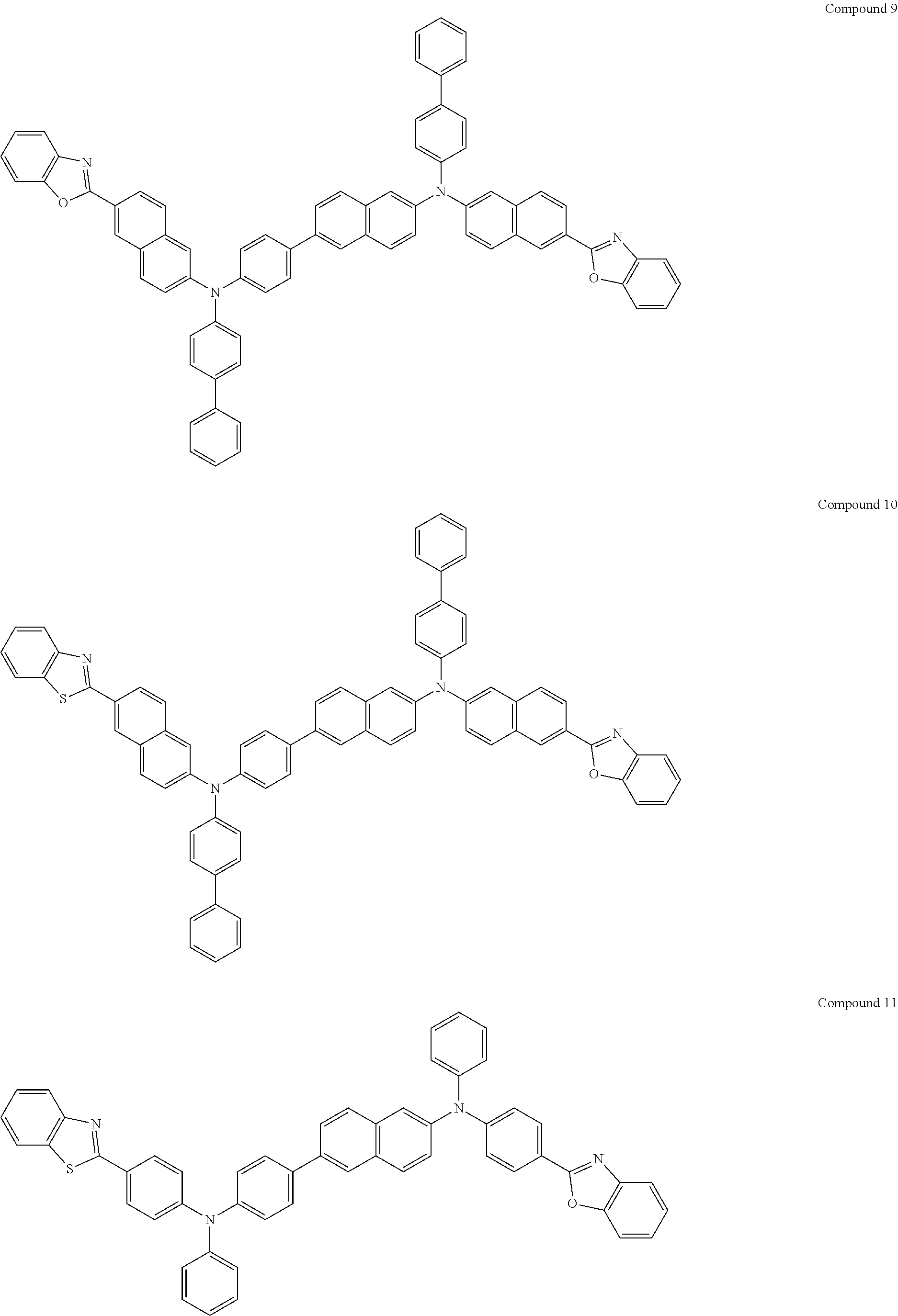 [SMILES: c1ccc(-c2ccc(N(c3ccc(-c4ccc5cc(N(c6ccc(-c7ccccc7)cc6)c6ccc7cc(-c8nc9ccccc9o8)ccc7c6)ccc5c4)cc3)c3ccc4cc(-c5nc6ccccc6o5)ccc4c3)cc2)cc1 ; c1ccc(-c2ccc(N(c3ccc4cc(-c5ccc(N(c6ccc(-c7ccccc7)cc6)c6ccc7cc(-c8nc9ccccc9s8)ccc7c6)cc5)ccc4c3)c3ccc4cc(-c5nc6ccccc6o5)ccc4c3)cc2)cc1 ; c1ccc(N(c2ccc(-c3ccc4cc(N(c5ccccc5)c5ccc(-c6nc7ccccc7o6)cc5)ccc4c3)cc2)c2ccc(-c3nc4ccccc4s3)cc2)cc1]

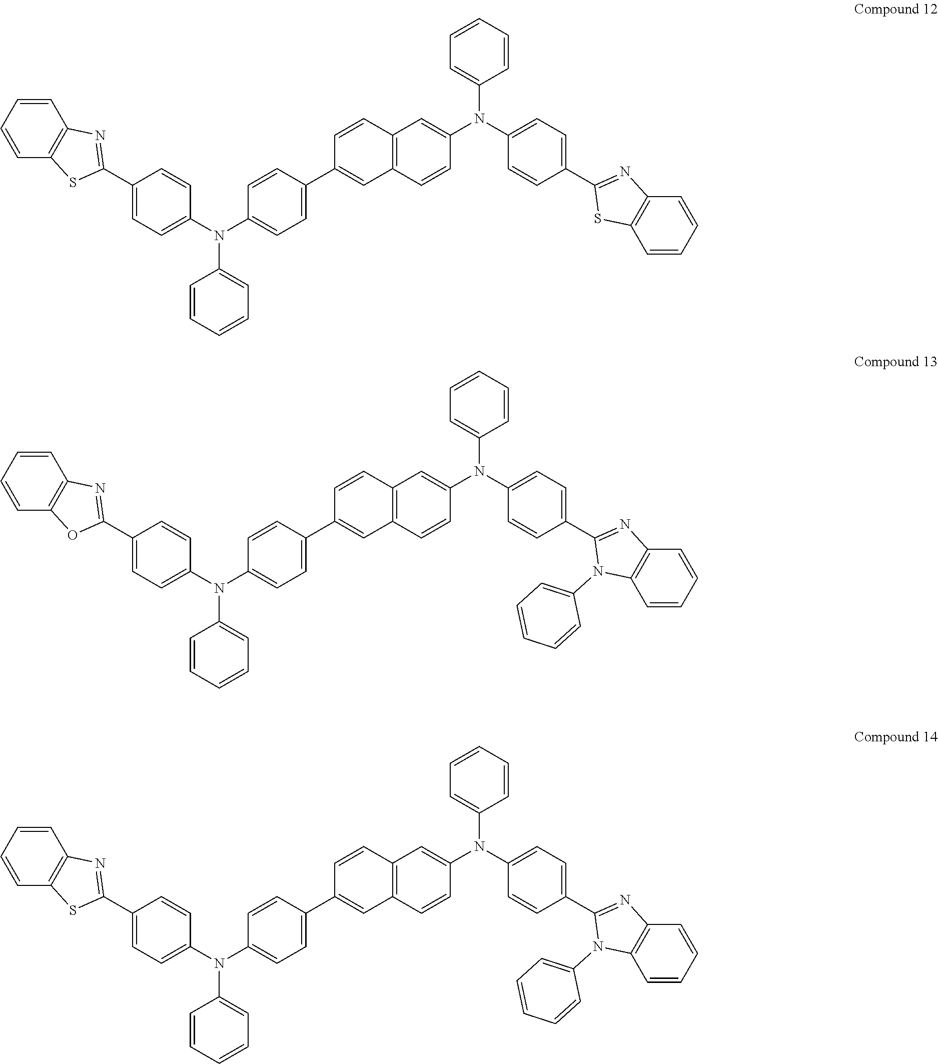 [SMILES: c1ccc(N(c2ccc(-c3ccc4cc(N(c5ccccc5)c5ccc(-c6nc7ccccc7s6)cc5)ccc4c3)cc2)c2ccc(-c3nc4ccccc4s3)cc2)cc1 ; c1ccc(N(c2ccc(-c3ccc4cc(N(c5ccccc5)c5ccc(-c6nc7ccccc7n6-c6ccccc6)cc5)ccc4c3)cc2)c2ccc(-c3nc4ccccc4o3)cc2)cc1 ; c1ccc(N(c2ccc(-c3ccc4cc(N(c5ccccc5)c5ccc(-c6nc7ccccc7n6-c6ccccc6)cc5)ccc4c3)cc2)c2ccc(-c3nc4ccccc4s3)cc2)cc1]

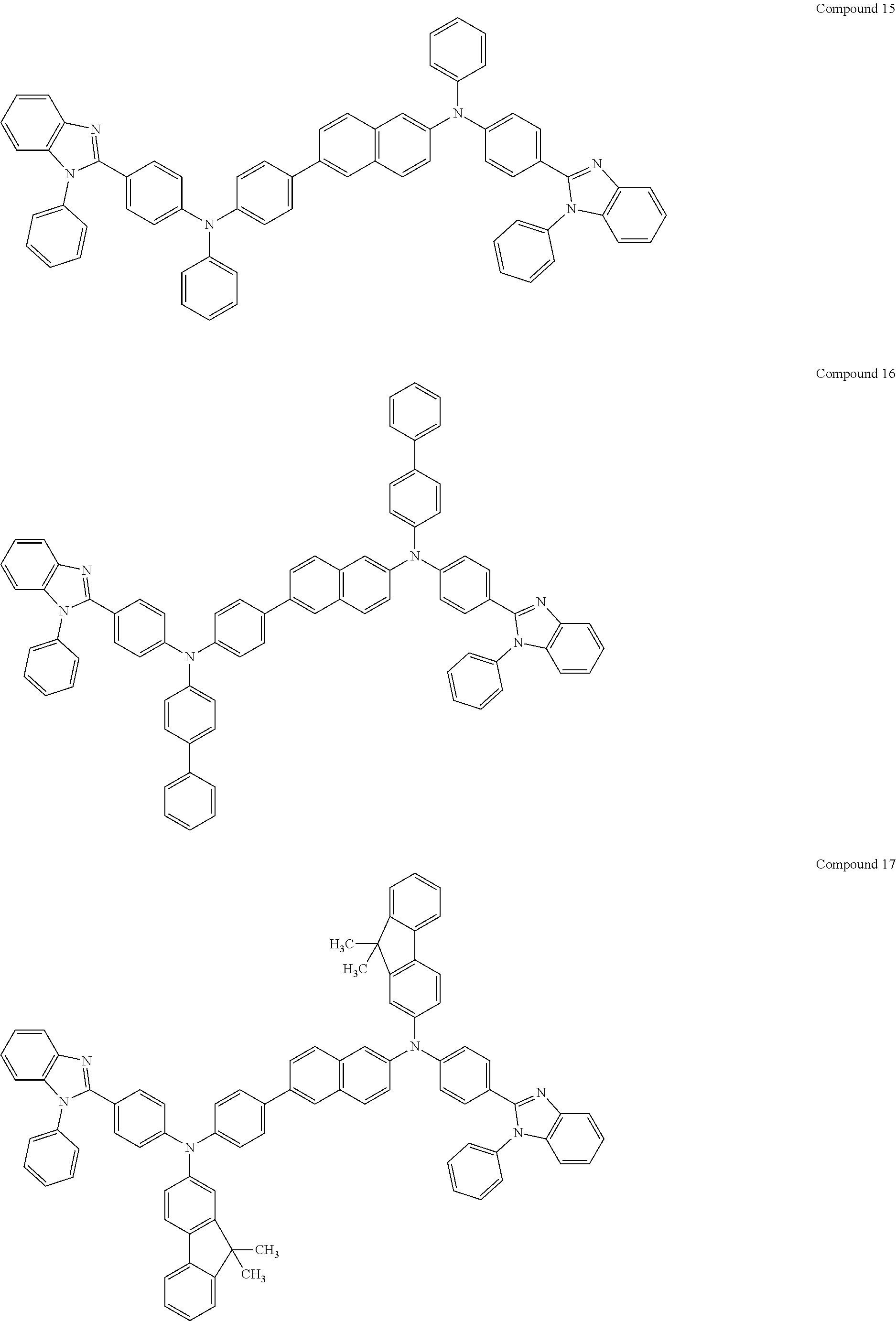 [SMILES: c1ccc(N(c2ccc(-c3ccc4cc(N(c5ccccc5)c5ccc(-c6nc7ccccc7n6-c6ccccc6)cc5)ccc4c3)cc2)c2ccc(-c3nc4ccccc4n3-c3ccccc3)cc2)cc1 ; c1ccc(-c2ccc(N(c3ccc(-c4ccc5cc(N(c6ccc(-c7ccccc7)cc6)c6ccc(-c7nc8ccccc8n7-c7ccccc7)cc6)ccc5c4)cc3)c3ccc(-c4nc5ccccc5n4-c4ccccc4)cc3)cc2)cc1 ; CC1(C)c2ccccc2-c2ccc(N(c3ccc(-c4ccc5cc(N(c6ccc(-c7nc8ccccc8n7-c7ccccc7)cc6)c6ccc7c(c6)C(C)(C)c6ccccc6-7)ccc5c4)cc3)c3ccc(-c4nc5ccccc5n4-c4ccccc4)cc3)cc21]

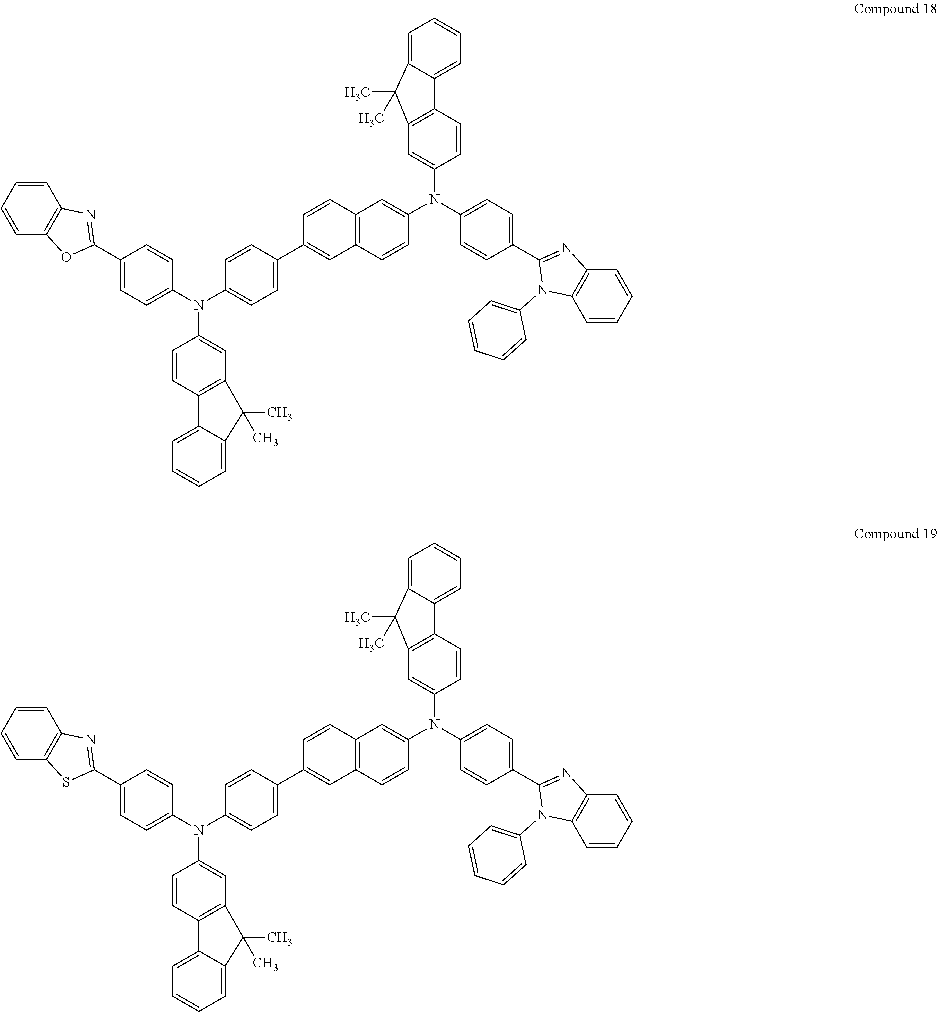 [SMILES: CC1(C)c2ccccc2-c2ccc(N(c3ccc(-c4ccc5cc(N(c6ccc(-c7nc8ccccc8n7-c7ccccc7)cc6)c6ccc7c(c6)C(C)(C)c6ccccc6-7)ccc5c4)cc3)c3ccc(-c4nc5ccccc5o4)cc3)cc21 ; CC1(C)c2ccccc2-c2ccc(N(c3ccc(-c4ccc5cc(N(c6ccc(-c7nc8ccccc8n7-c7ccccc7)cc6)c6ccc7c(c6)C(C)(C)c6ccccc6-7)ccc5c4)cc3)c3ccc(-c4nc5ccccc5s4)cc3)cc21]

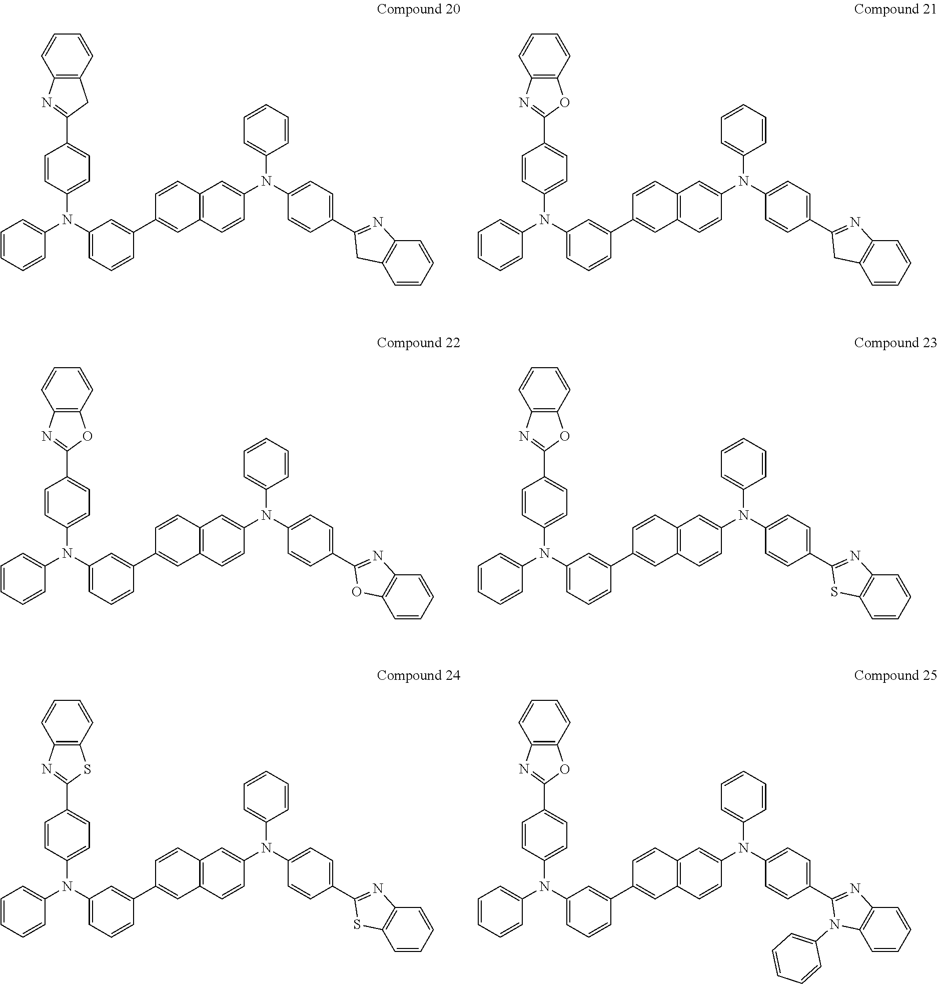

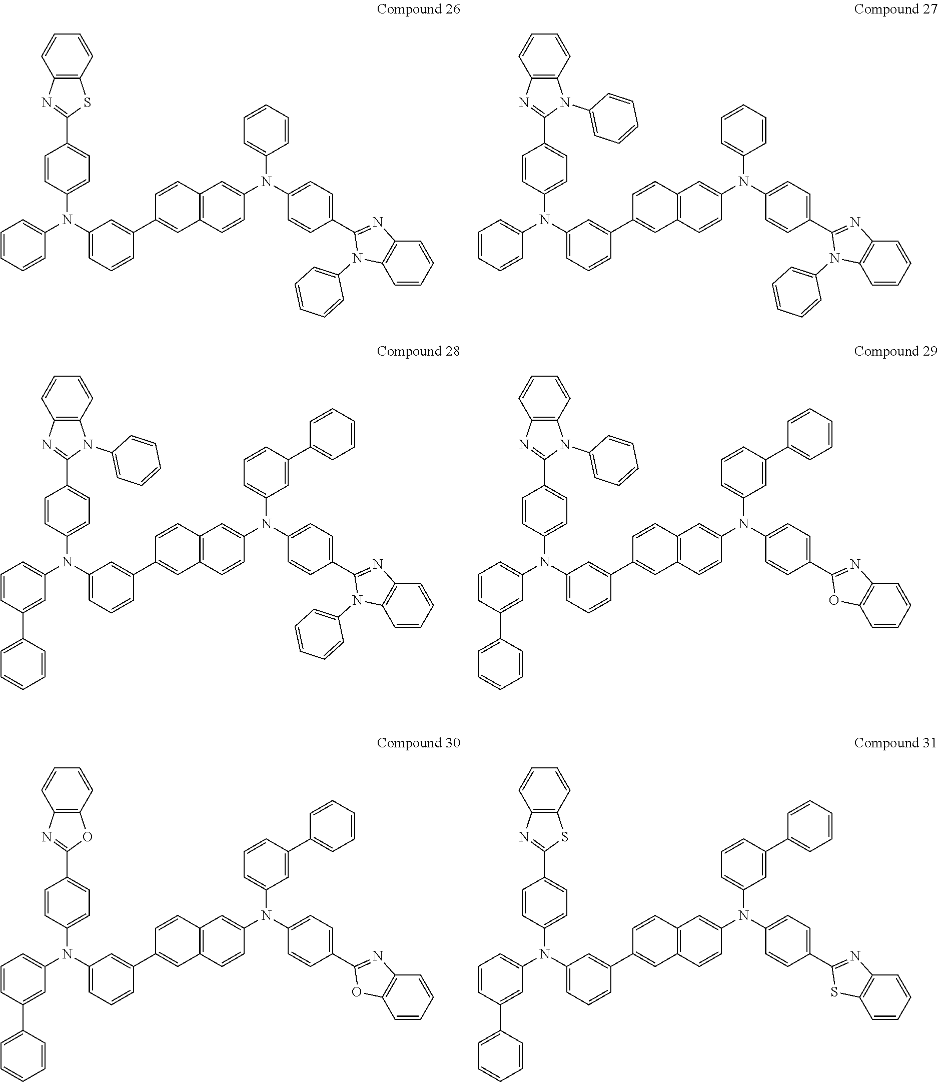

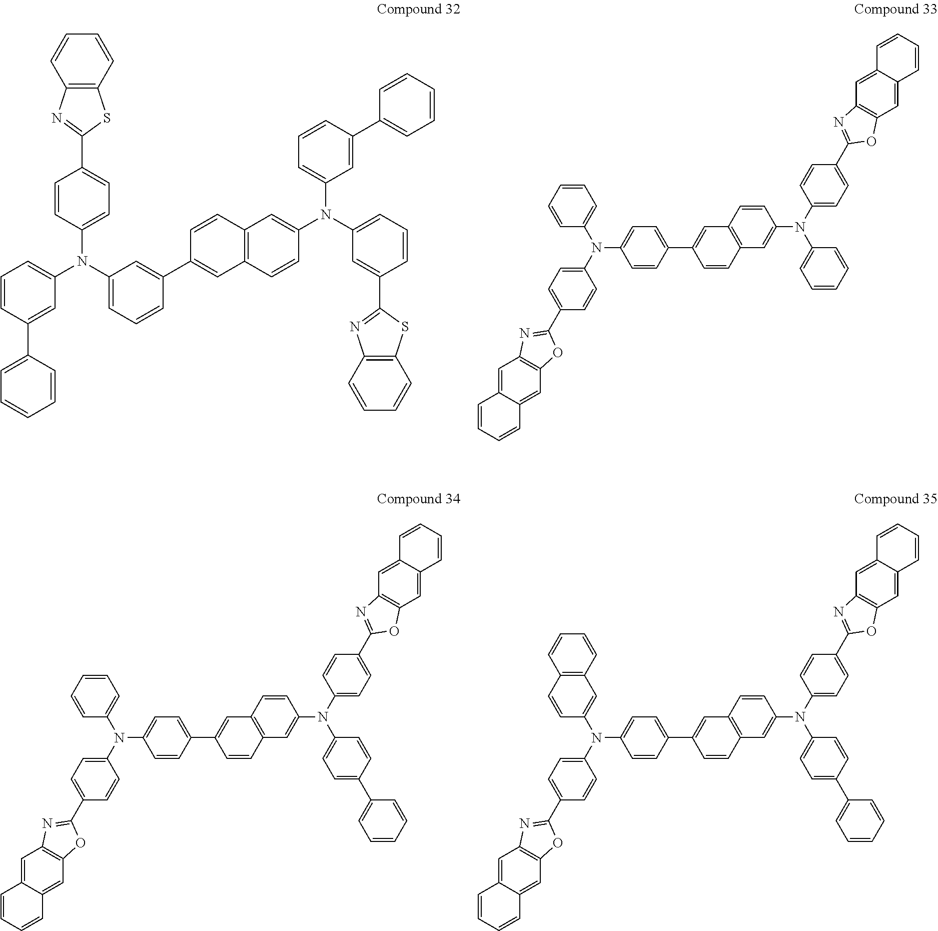 [SMILES: c1ccc(-c2cccc(N(c3ccc(-c4nc5ccccc5s4)cc3)c3cccc(-c4ccc5cc(N(c6cccc(-c7ccccc7)c6)c6cccc(-c7nc8ccccc8s7)c6)ccc5c4)c3)c2)cc1 ; c1ccc(N(c2ccc(-c3ccc4cc(N(c5ccccc5)c5ccc(-c6nc7cc8ccccc8cc7o6)cc5)ccc4c3)cc2)c2ccc(-c3nc4cc5ccccc5cc4o3)cc2)cc1 ; c1ccc(-c2ccc(N(c3ccc(-c4nc5cc6ccccc6cc5o4)cc3)c3ccc4cc(-c5ccc(N(c6ccccc6)c6ccc(-c7nc8cc9ccccc9cc8o7)cc6)cc5)ccc4c3)cc2)cc1 ; c1ccc(-c2ccc(N(c3ccc(-c4nc5cc6ccccc6cc5o4)cc3)c3ccc4cc(-c5ccc(N(c6ccc(-c7nc8cc9ccccc9cc8o7)cc6)c6ccc7ccccc7c6)cc5)ccc4c3)cc2)cc1]

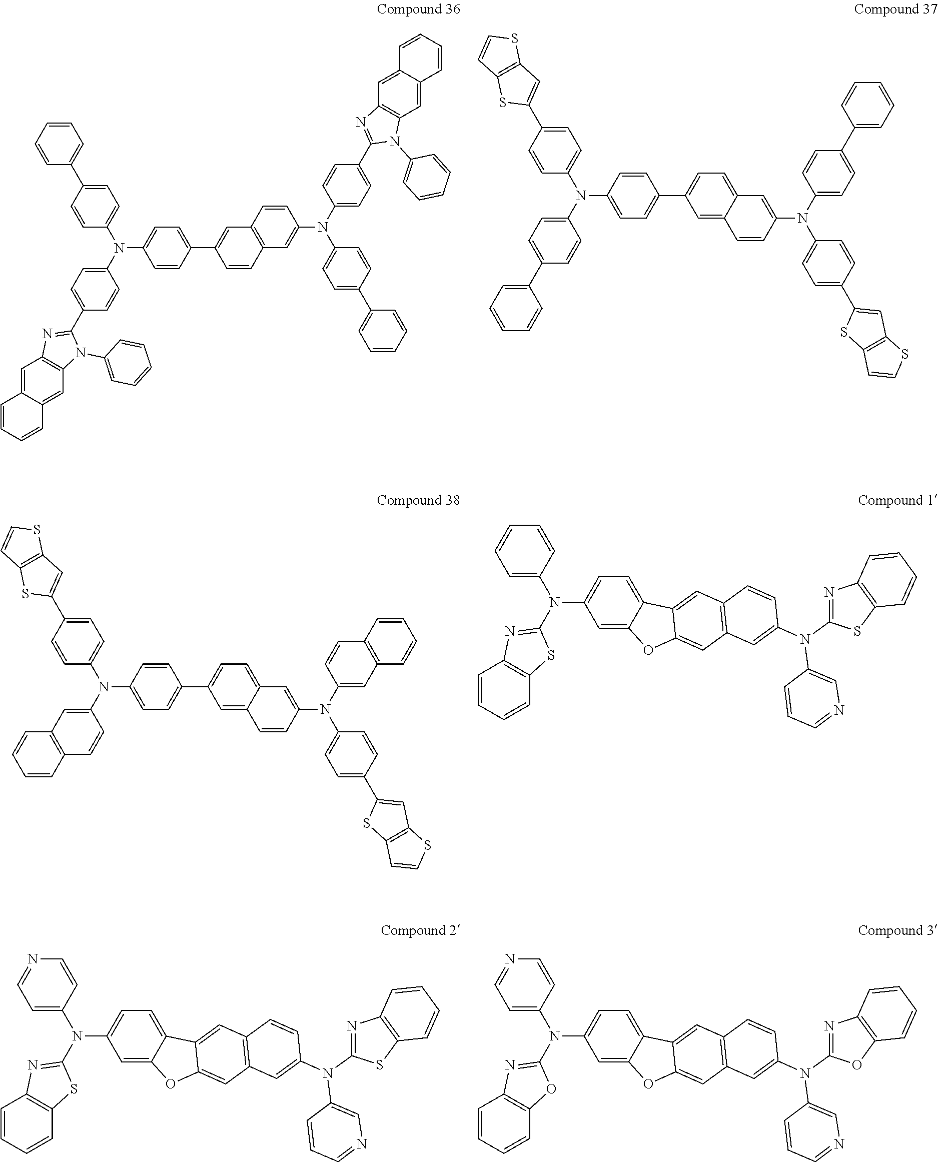

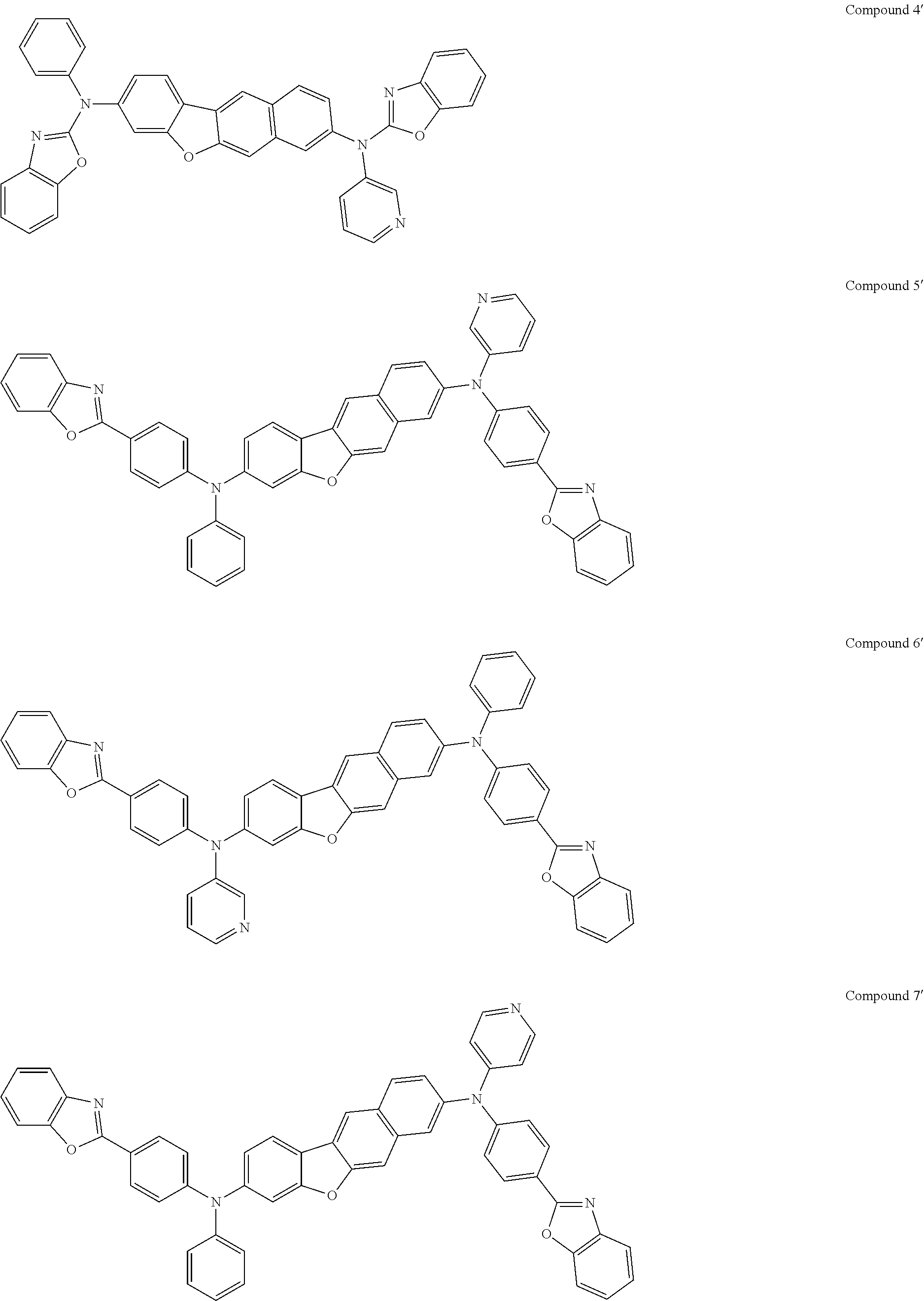 [SMILES: c1ccc(N(c2ccc3c(c2)oc2cc4cc(N(c5cccnc5)c5nc6ccccc6o5)ccc4cc23)c2nc3ccccc3o2)cc1 ; c1ccc(N(c2ccc(-c3nc4ccccc4o3)cc2)c2ccc3c(c2)oc2cc4cc(N(c5ccc(-c6nc7ccccc7o6)cc5)c5cccnc5)ccc4cc23)cc1 ; c1ccc(N(c2ccc(-c3nc4ccccc4o3)cc2)c2ccc3cc4c(cc3c2)oc2cc(N(c3ccc(-c5nc6ccccc6o5)cc3)c3cccnc3)ccc24)cc1 ; c1ccc(N(c2ccc(-c3nc4ccccc4o3)cc2)c2ccc3c(c2)oc2cc4cc(N(c5ccncc5)c5ccc(-c6nc7ccccc7o6)cc5)ccc4cc23)cc1]

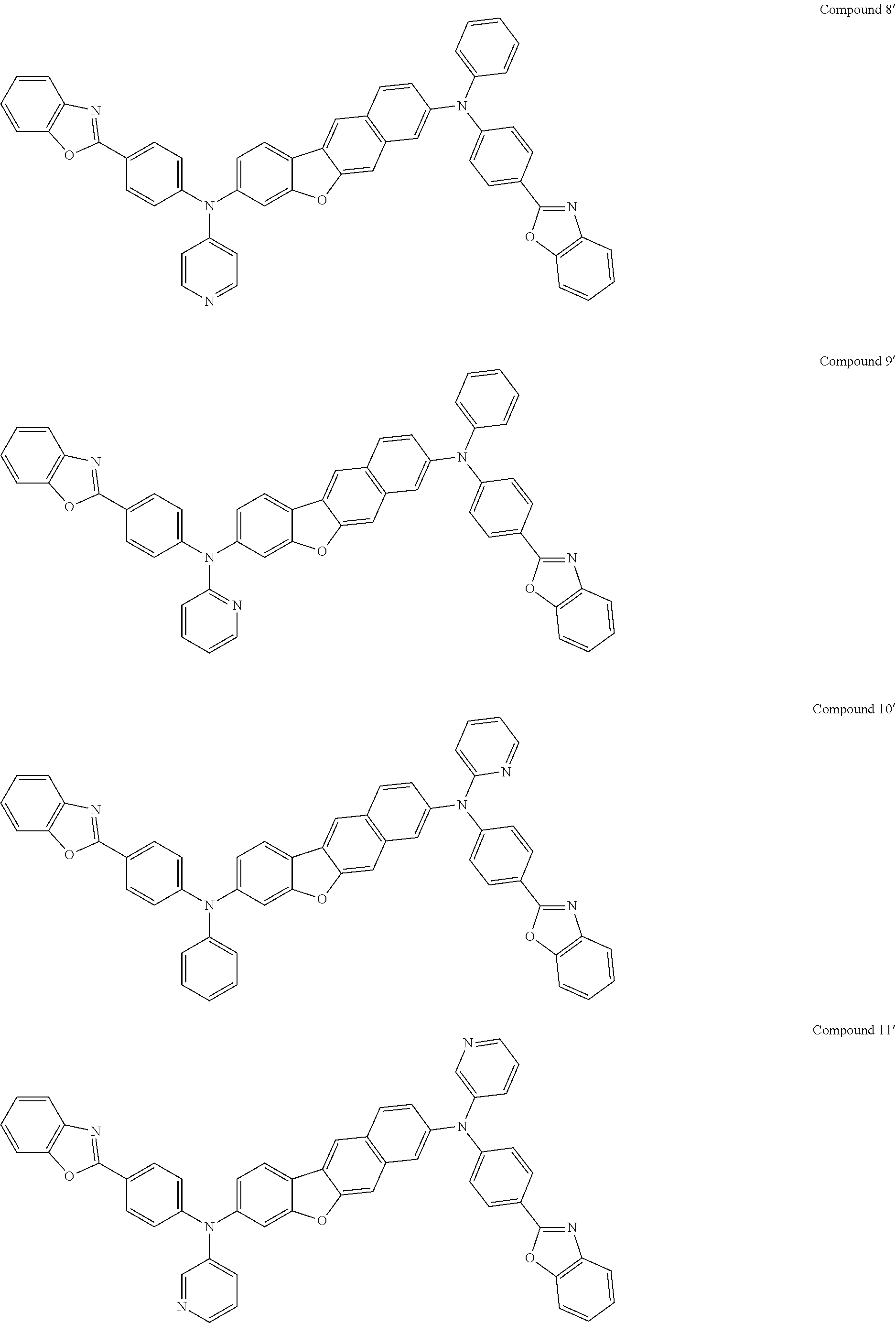 [SMILES: c1ccc(N(c2ccc(-c3nc4ccccc4o3)cc2)c2ccc3cc4c(cc3c2)oc2cc(N(c3ccncc3)c3ccc(-c5nc6ccccc6o5)cc3)ccc24)cc1 ; c1ccc(N(c2ccc(-c3nc4ccccc4o3)cc2)c2ccc3cc4c(cc3c2)oc2cc(N(c3ccc(-c5nc6ccccc6o5)cc3)c3ccccn3)ccc24)cc1 ; c1ccc(N(c2ccc(-c3nc4ccccc4o3)cc2)c2ccc3c(c2)oc2cc4cc(N(c5ccc(-c6nc7ccccc7o6)cc5)c5ccccn5)ccc4cc23)cc1 ; c1cncc(N(c2ccc(-c3nc4ccccc4o3)cc2)c2ccc3cc4c(cc3c2)oc2cc(N(c3ccc(-c5nc6ccccc6o5)cc3)c3cccnc3)ccc24)c1]

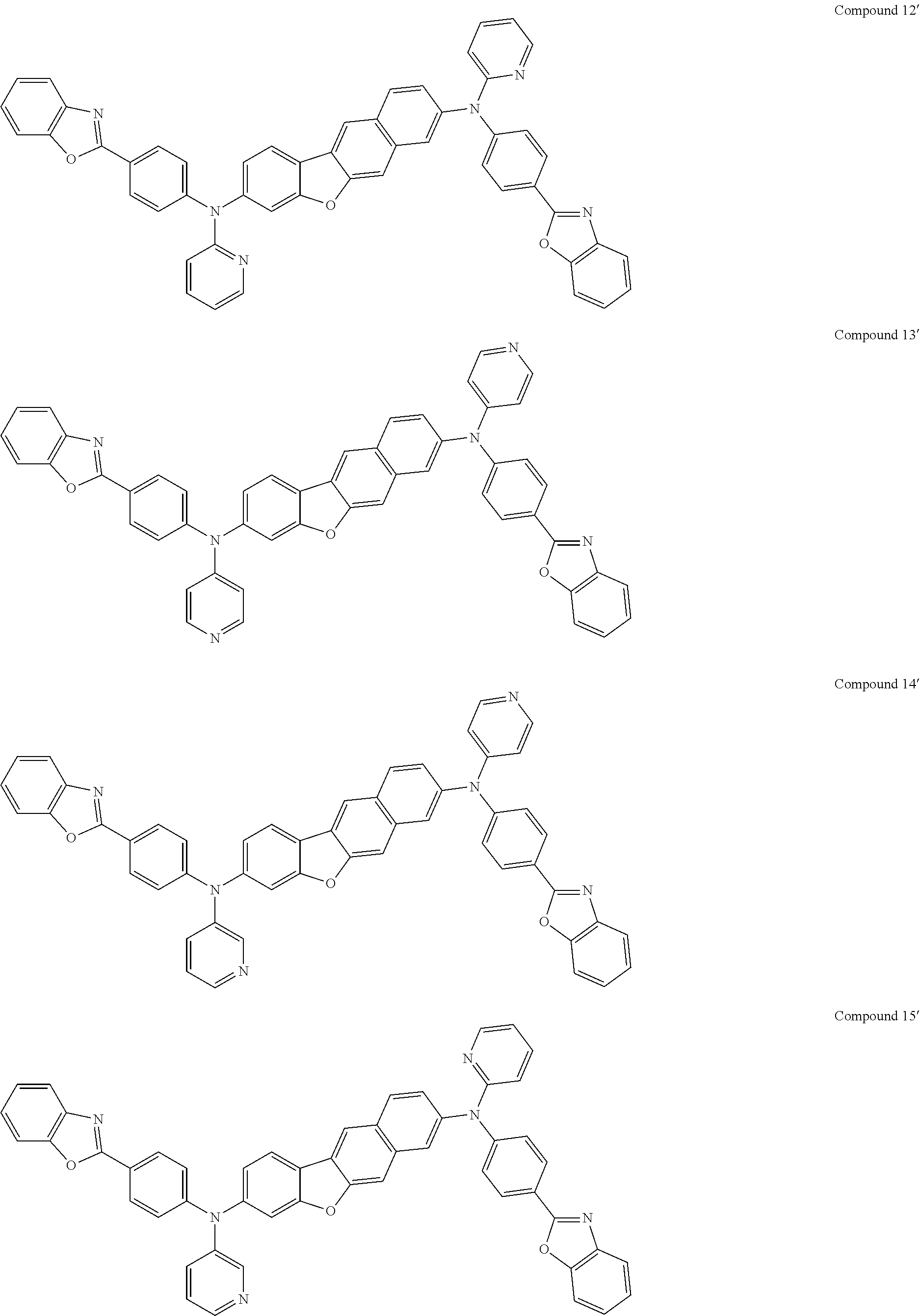 [SMILES: c1ccc(N(c2ccc(-c3nc4ccccc4o3)cc2)c2ccc3cc4c(cc3c2)oc2cc(N(c3ccc(-c5nc6ccccc6o5)cc3)c3ccccn3)ccc24)nc1 ; c1ccc2oc(-c3ccc(N(c4ccncc4)c4ccc5cc6c(cc5c4)oc4cc(N(c5ccncc5)c5ccc(-c7nc8ccccc8o7)cc5)ccc46)cc3)nc2c1 ; c1cncc(N(c2ccc(-c3nc4ccccc4o3)cc2)c2ccc3c(c2)oc2cc4cc(N(c5ccncc5)c5ccc(-c6nc7ccccc7o6)cc5)ccc4cc23)c1 ; c1ccc(N(c2ccc(-c3nc4ccccc4o3)cc2)c2ccc3cc4c(cc3c2)oc2cc(N(c3ccc(-c5nc6ccccc6o5)cc3)c3cccnc3)ccc24)nc1]

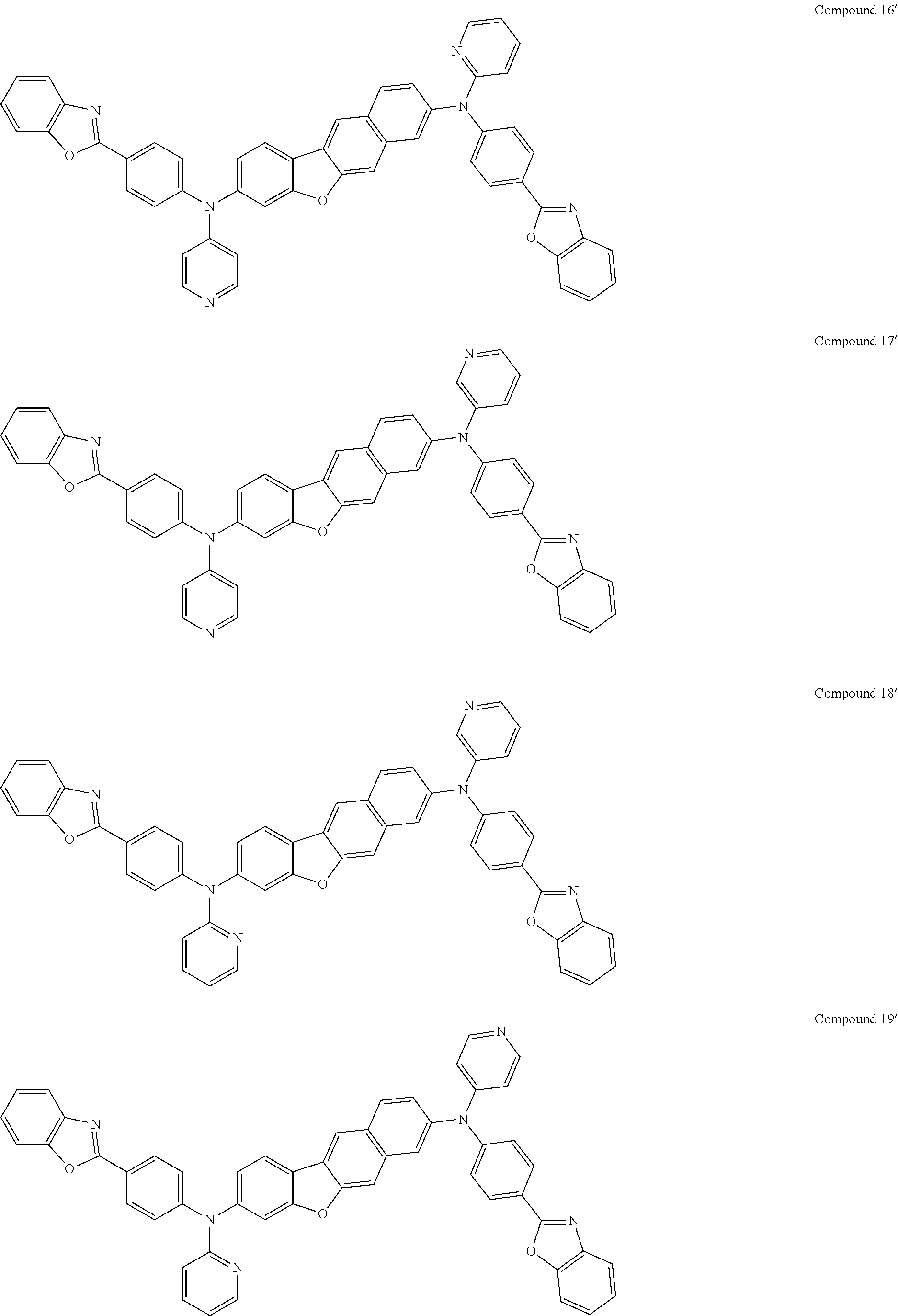 [SMILES: c1ccc(N(c2ccc(-c3nc4ccccc4o3)cc2)c2ccc3cc4c(cc3c2)oc2cc(N(c3ccncc3)c3ccc(-c5nc6ccccc6o5)cc3)ccc24)nc1 ; c1cncc(N(c2ccc(-c3nc4ccccc4o3)cc2)c2ccc3cc4c(cc3c2)oc2cc(N(c3ccncc3)c3ccc(-c5nc6ccccc6o5)cc3)ccc24)c1 ; c1ccc(N(c2ccc(-c3nc4ccccc4o3)cc2)c2ccc3c(c2)oc2cc4cc(N(c5ccc(-c6nc7ccccc7o6)cc5)c5cccnc5)ccc4cc23)nc1 ; c1ccc(N(c2ccc(-c3nc4ccccc4o3)cc2)c2ccc3c(c2)oc2cc4cc(N(c5ccncc5)c5ccc(-c6nc7ccccc7o6)cc5)ccc4cc23)nc1]

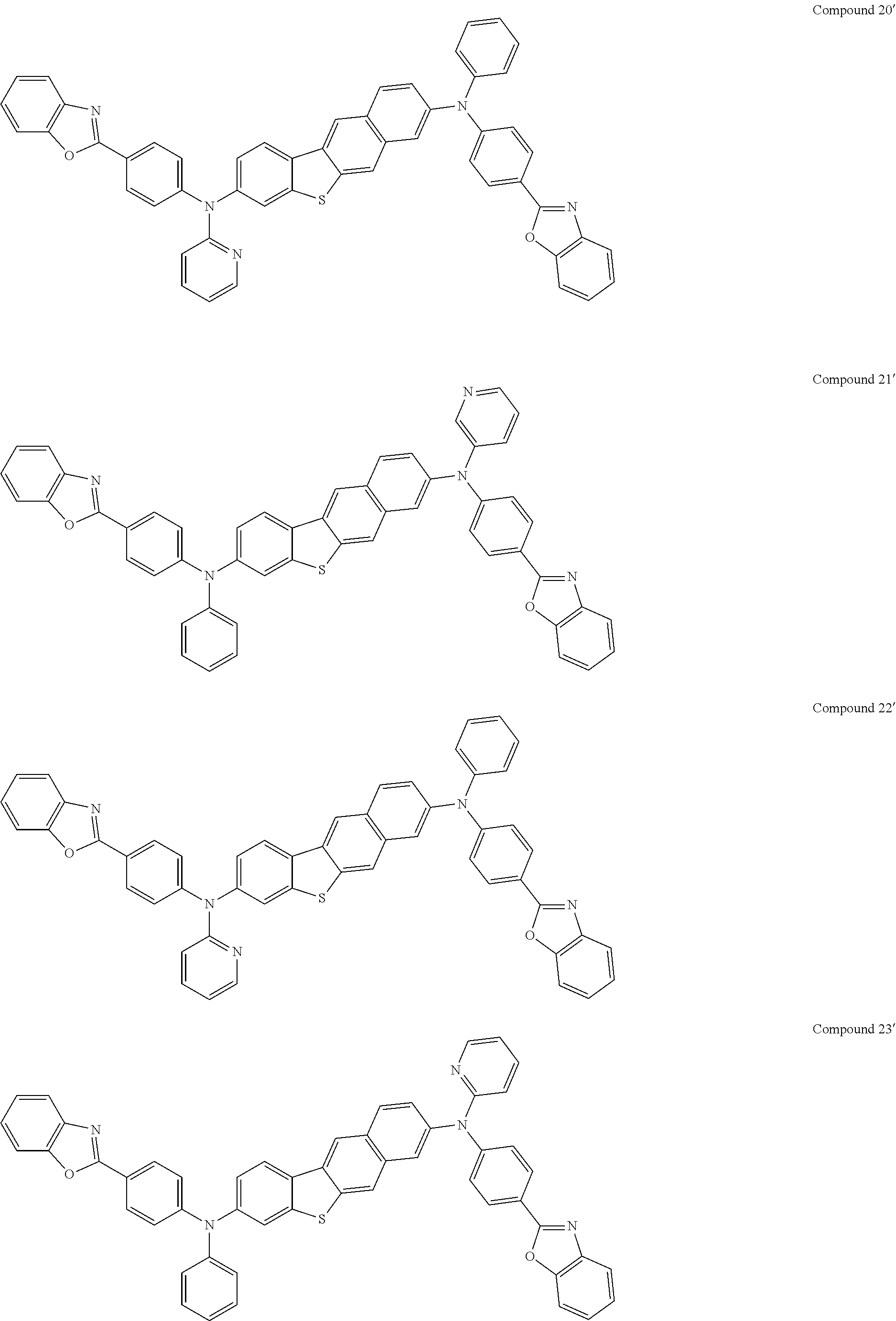 [SMILES: c1ccc(N(c2ccc(-c3nc4ccccc4o3)cc2)c2ccc3cc4c(cc3c2)sc2cc(N(c3ccc(-c5nc6ccccc6o5)cc3)c3ccccn3)ccc24)cc1 ; c1ccc(N(c2ccc(-c3nc4ccccc4o3)cc2)c2ccc3c(c2)sc2cc4cc(N(c5ccc(-c6nc7ccccc7o6)cc5)c5cccnc5)ccc4cc23)cc1 ; c1ccc(N(c2ccc(-c3nc4ccccc4o3)cc2)c2ccc3cc4c(cc3c2)sc2cc(N(c3ccc(-c5nc6ccccc6o5)cc3)c3ccccn3)ccc24)cc1 ; c1ccc(N(c2ccc(-c3nc4ccccc4o3)cc2)c2ccc3c(c2)sc2cc4cc(N(c5ccc(-c6nc7ccccc7o6)cc5)c5ccccn5)ccc4cc23)cc1]

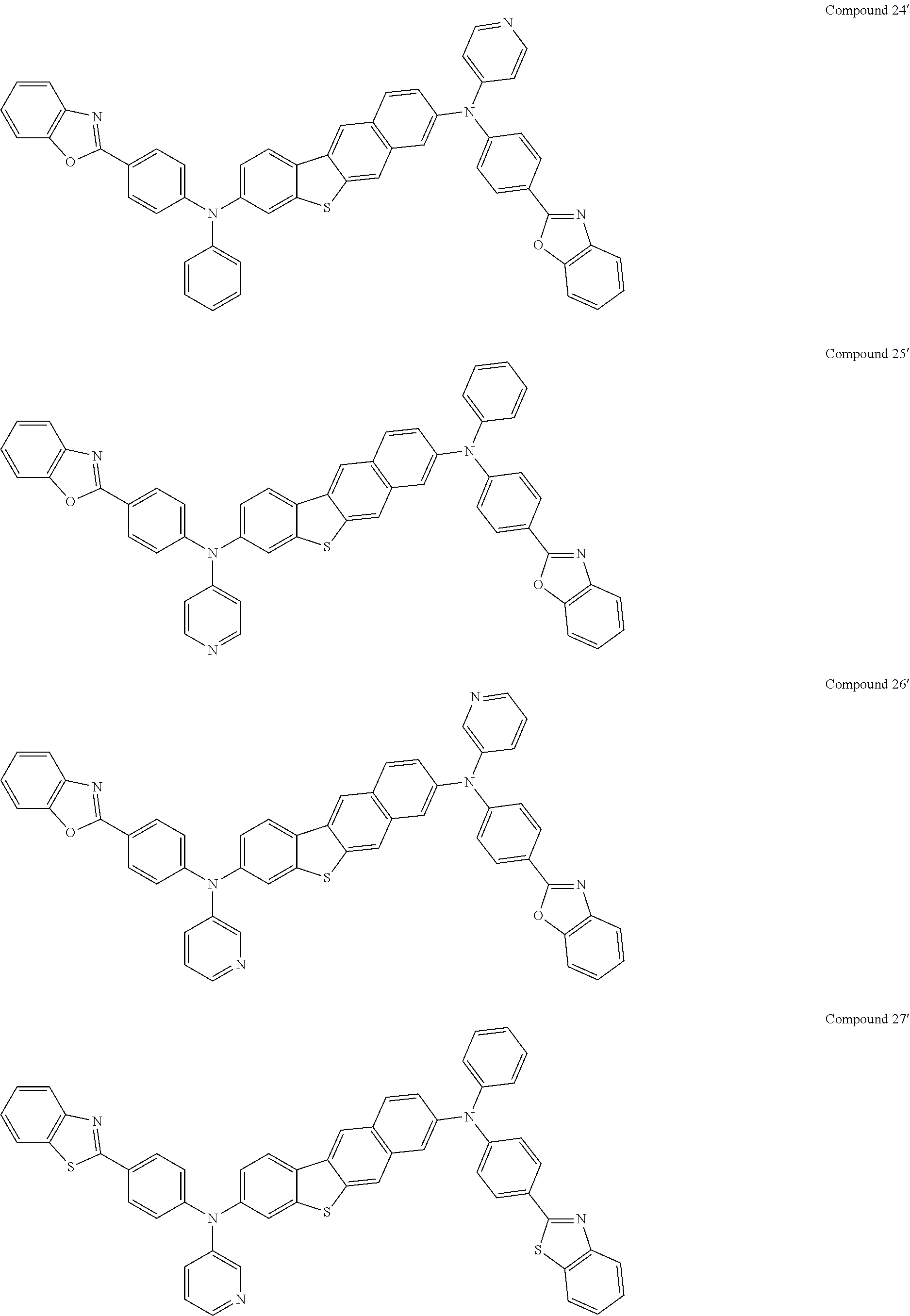 [SMILES: c1ccc(N(c2ccc(-c3nc4ccccc4o3)cc2)c2ccc3c(c2)sc2cc4cc(N(c5ccncc5)c5ccc(-c6nc7ccccc7o6)cc5)ccc4cc23)cc1 ; c1ccc(N(c2ccc(-c3nc4ccccc4o3)cc2)c2ccc3cc4c(cc3c2)sc2cc(N(c3ccncc3)c3ccc(-c5nc6ccccc6o5)cc3)ccc24)cc1 ; c1cncc(N(c2ccc(-c3nc4ccccc4o3)cc2)c2ccc3cc4c(cc3c2)sc2cc(N(c3ccc(-c5nc6ccccc6o5)cc3)c3cccnc3)ccc24)c1 ; c1ccc(N(c2ccc(-c3nc4ccccc4s3)cc2)c2ccc3cc4c(cc3c2)sc2cc(N(c3ccc(-c5nc6ccccc6s5)cc3)c3cccnc3)ccc24)cc1]

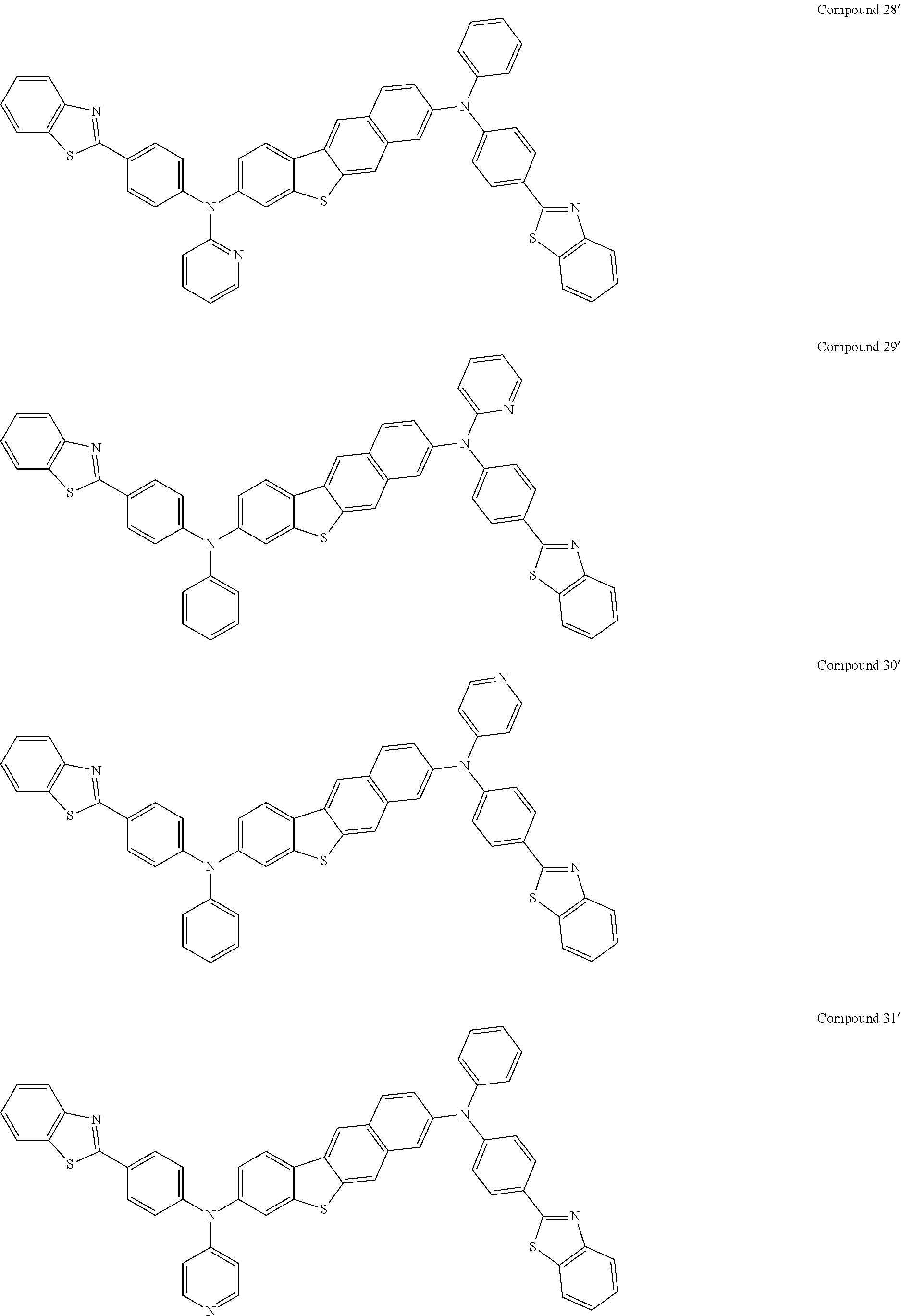 [SMILES: c1ccc(N(c2ccc(-c3nc4ccccc4s3)cc2)c2ccc3cc4c(cc3c2)sc2cc(N(c3ccc(-c5nc6ccccc6s5)cc3)c3ccccn3)ccc24)cc1 ; c1ccc(N(c2ccc(-c3nc4ccccc4s3)cc2)c2ccc3c(c2)sc2cc4cc(N(c5ccc(-c6nc7ccccc7s6)cc5)c5ccccn5)ccc4cc23)cc1 ; c1ccc(N(c2ccc(-c3nc4ccccc4s3)cc2)c2ccc3c(c2)sc2cc4cc(N(c5ccncc5)c5ccc(-c6nc7ccccc7s6)cc5)ccc4cc23)cc1 ; c1ccc(N(c2ccc(-c3nc4ccccc4s3)cc2)c2ccc3cc4c(cc3c2)sc2cc(N(c3ccncc3)c3ccc(-c5nc6ccccc6s5)cc3)ccc24)cc1]

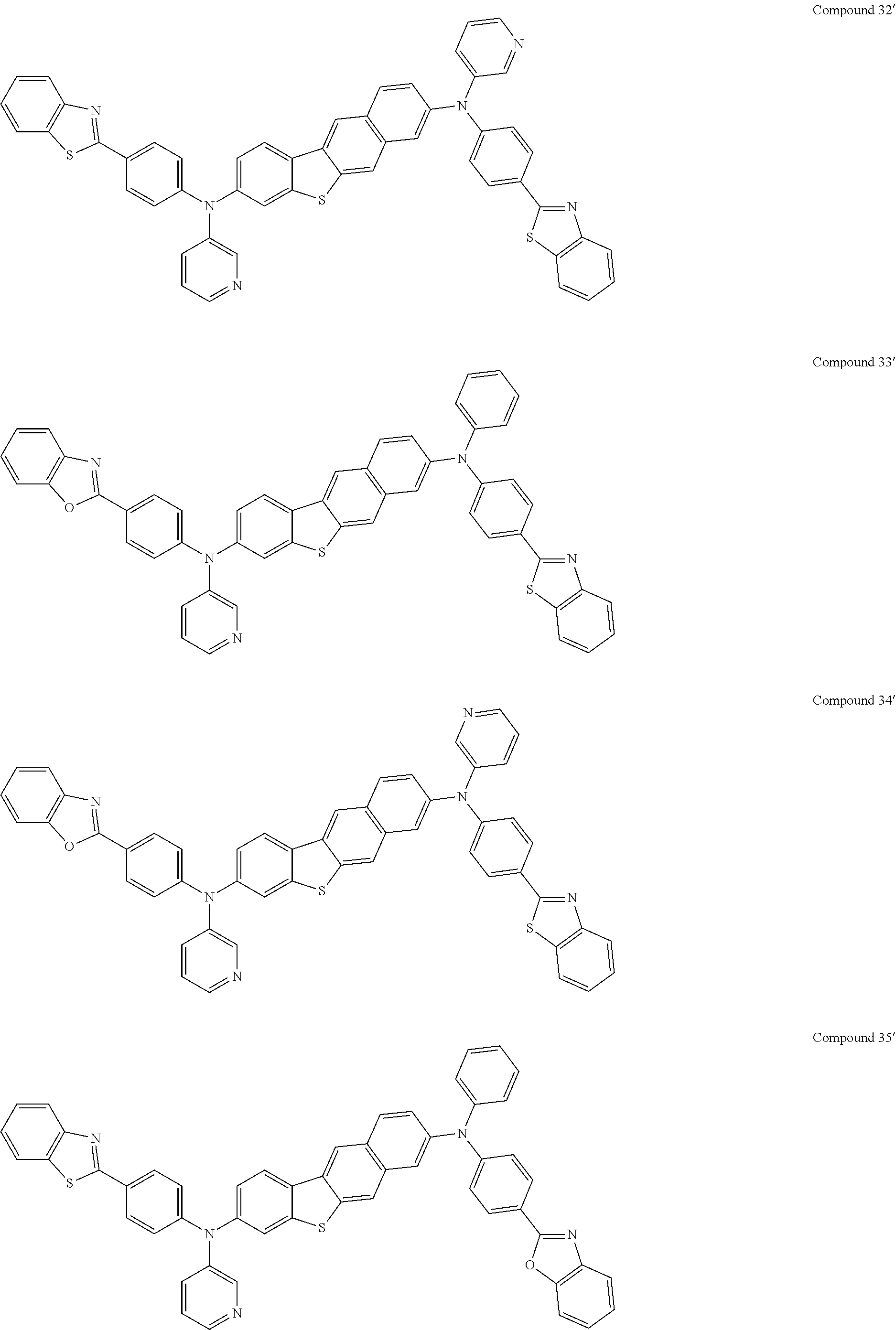 [SMILES: c1cncc(N(c2ccc(-c3nc4ccccc4s3)cc2)c2ccc3cc4c(cc3c2)sc2cc(N(c3ccc(-c5nc6ccccc6s5)cc3)c3cccnc3)ccc24)c1 ; c1ccc(N(c2ccc(-c3nc4ccccc4s3)cc2)c2ccc3cc4c(cc3c2)sc2cc(N(c3ccc(-c5nc6ccccc6o5)cc3)c3cccnc3)ccc24)cc1 ; c1cncc(N(c2ccc(-c3nc4ccccc4s3)cc2)c2ccc3cc4c(cc3c2)sc2cc(N(c3ccc(-c5nc6ccccc6o5)cc3)c3cccnc3)ccc24)c1 ; c1ccc(N(c2ccc(-c3nc4ccccc4o3)cc2)c2ccc3cc4c(cc3c2)sc2cc(N(c3ccc(-c5nc6ccccc6s5)cc3)c3cccnc3)ccc24)cc1]

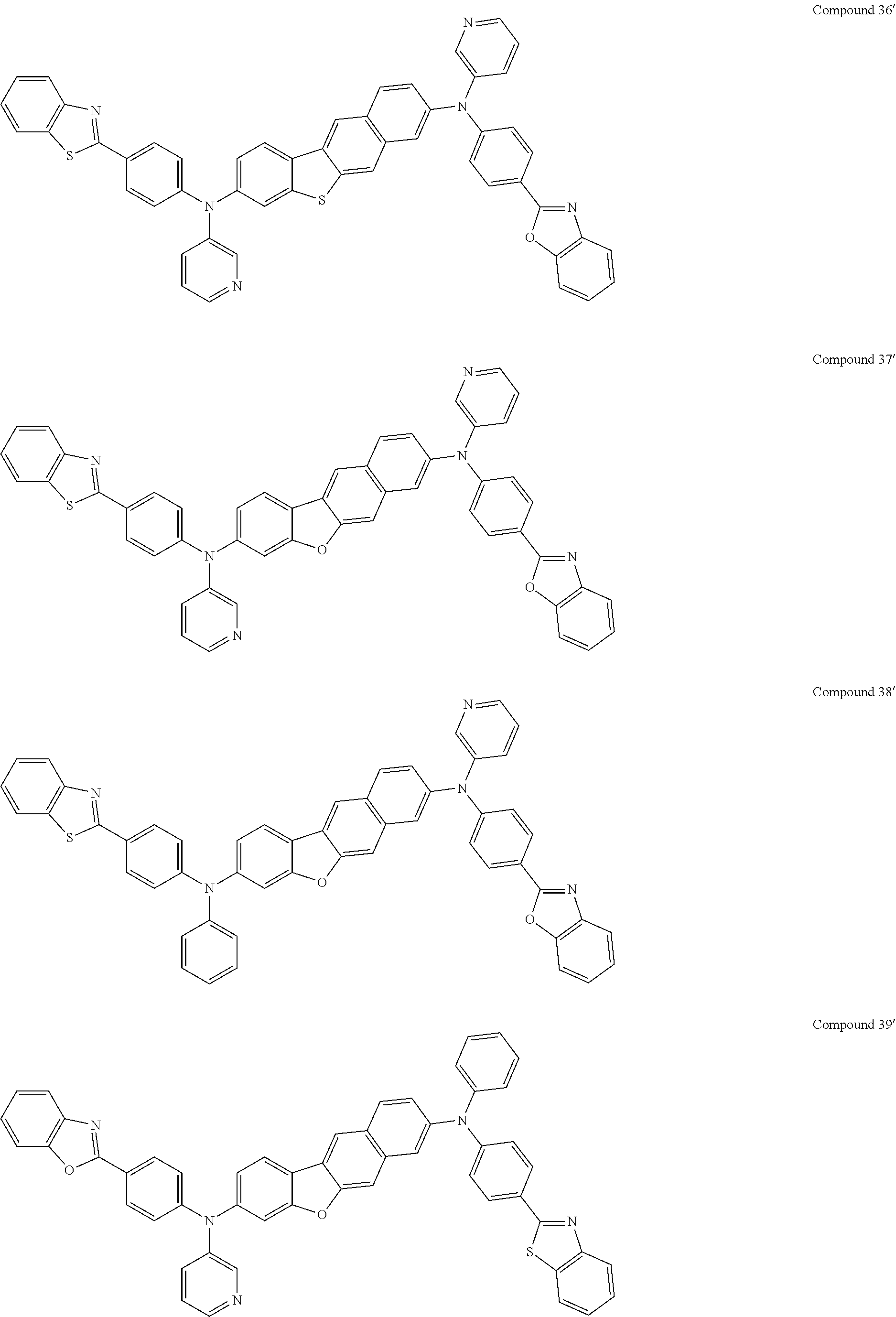 [SMILES: c1cncc(N(c2ccc(-c3nc4ccccc4o3)cc2)c2ccc3cc4c(cc3c2)sc2cc(N(c3ccc(-c5nc6ccccc6s5)cc3)c3cccnc3)ccc24)c1 ; c1cncc(N(c2ccc(-c3nc4ccccc4o3)cc2)c2ccc3cc4c(cc3c2)oc2cc(N(c3ccc(-c5nc6ccccc6s5)cc3)c3cccnc3)ccc24)c1 ; c1ccc(N(c2ccc(-c3nc4ccccc4s3)cc2)c2ccc3c(c2)oc2cc4cc(N(c5ccc(-c6nc7ccccc7o6)cc5)c5cccnc5)ccc4cc23)cc1 ; c1ccc(N(c2ccc(-c3nc4ccccc4s3)cc2)c2ccc3cc4c(cc3c2)oc2cc(N(c3ccc(-c5nc6ccccc6o5)cc3)c3cccnc3)ccc24)cc1]

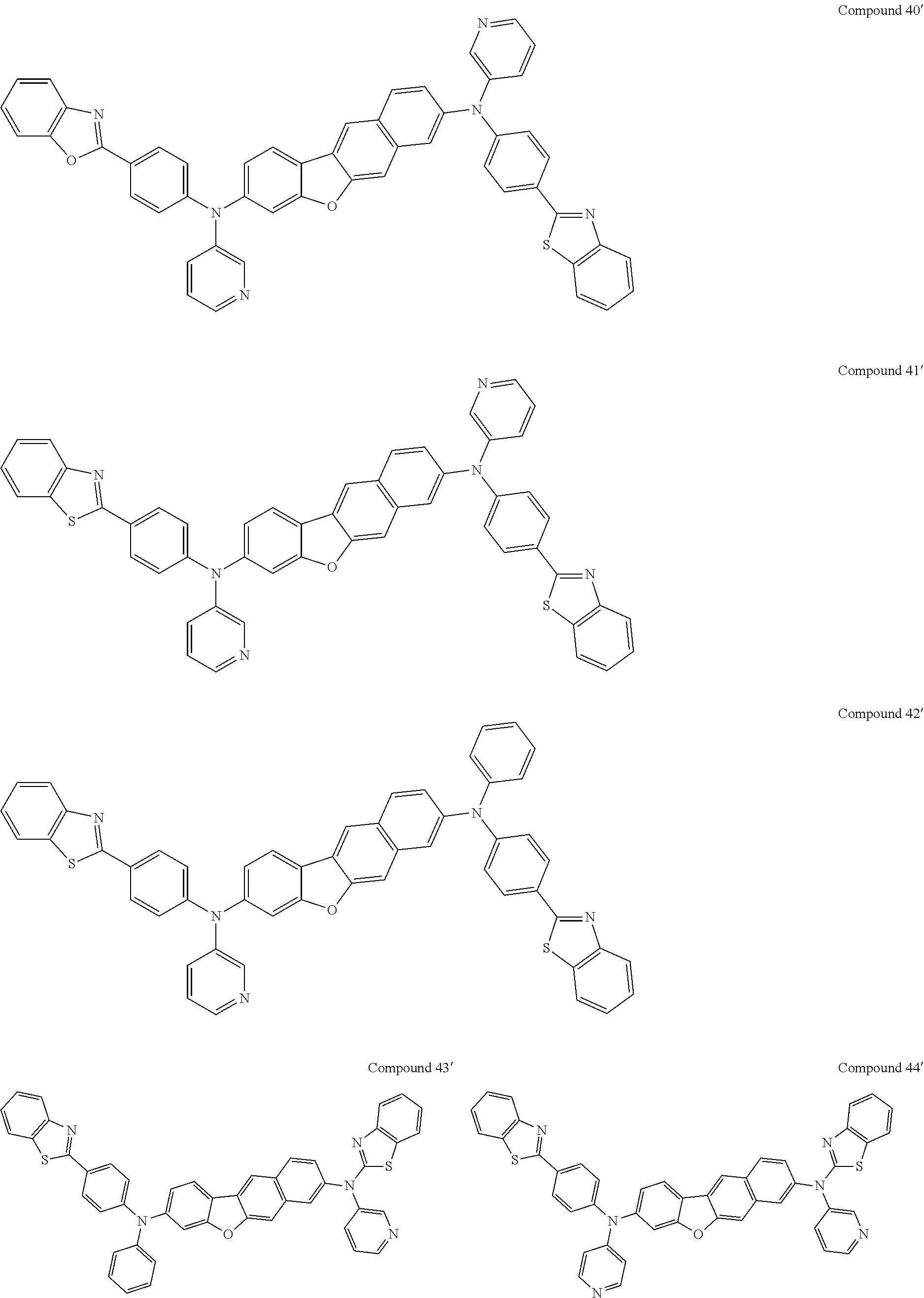 [SMILES: c1cncc(N(c2ccc(-c3nc4ccccc4s3)cc2)c2ccc3cc4c(cc3c2)oc2cc(N(c3ccc(-c5nc6ccccc6o5)cc3)c3cccnc3)ccc24)c1 ; c1cncc(N(c2ccc(-c3nc4ccccc4s3)cc2)c2ccc3cc4c(cc3c2)oc2cc(N(c3ccc(-c5nc6ccccc6s5)cc3)c3cccnc3)ccc24)c1 ; c1ccc(N(c2ccc(-c3nc4ccccc4s3)cc2)c2ccc3cc4c(cc3c2)oc2cc(N(c3ccc(-c5nc6ccccc6s5)cc3)c3cccnc3)ccc24)cc1 ; c1ccc(N(c2ccc(-c3nc4ccccc4s3)cc2)c2ccc3c(c2)oc2cc4cc(N(c5cccnc5)c5nc6ccccc6s5)ccc4cc23)cc1 ; c1cncc(N(c2ccc3cc4c(cc3c2)oc2cc(N(c3ccncc3)c3ccc(-c5nc6ccccc6s5)cc3)ccc24)c2nc3ccccc3s2)c1]

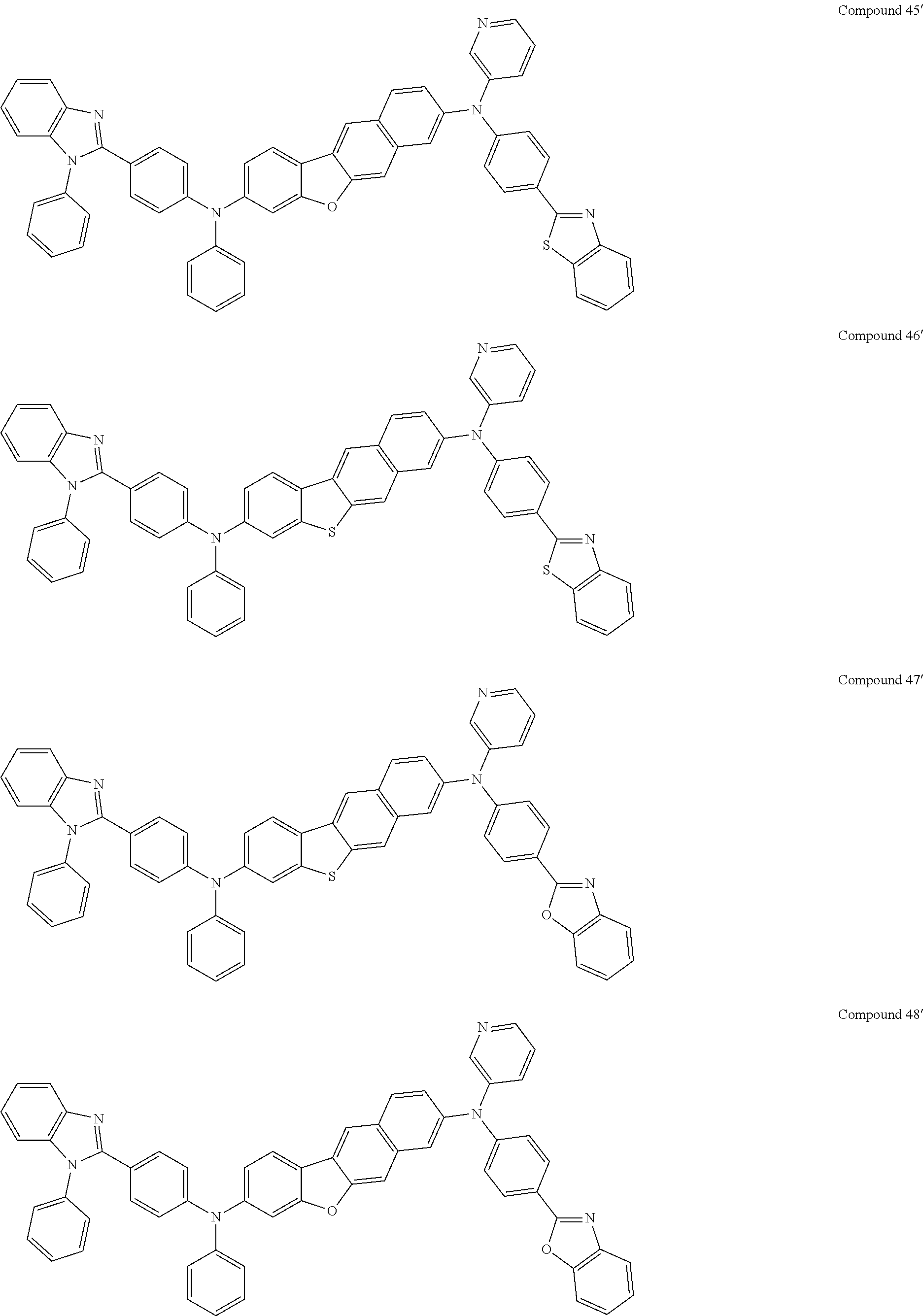 [SMILES: c1ccc(N(c2ccc(-c3nc4ccccc4n3-c3ccccc3)cc2)c2ccc3c(c2)oc2cc4cc(N(c5ccc(-c6nc7ccccc7s6)cc5)c5cccnc5)ccc4cc23)cc1 ; c1ccc(N(c2ccc(-c3nc4ccccc4n3-c3ccccc3)cc2)c2ccc3c(c2)sc2cc4cc(N(c5ccc(-c6nc7ccccc7s6)cc5)c5cccnc5)ccc4cc23)cc1 ; c1ccc(N(c2ccc(-c3nc4ccccc4n3-c3ccccc3)cc2)c2ccc3c(c2)sc2cc4cc(N(c5ccc(-c6nc7ccccc7o6)cc5)c5cccnc5)ccc4cc23)cc1 ; c1ccc(N(c2ccc(-c3nc4ccccc4n3-c3ccccc3)cc2)c2ccc3c(c2)oc2cc4cc(N(c5ccc(-c6nc7ccccc7o6)cc5)c5cccnc5)ccc4cc23)cc1]

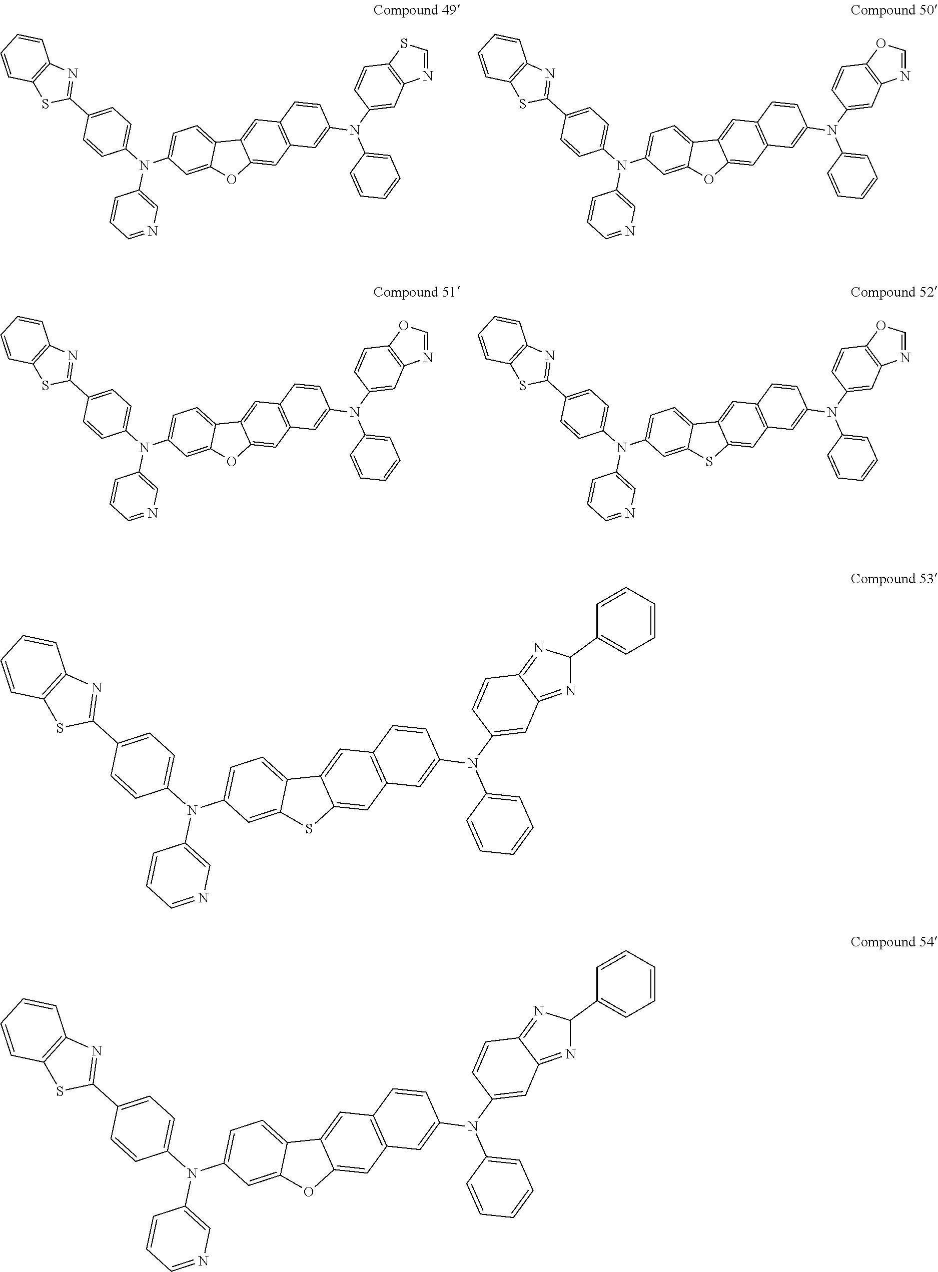

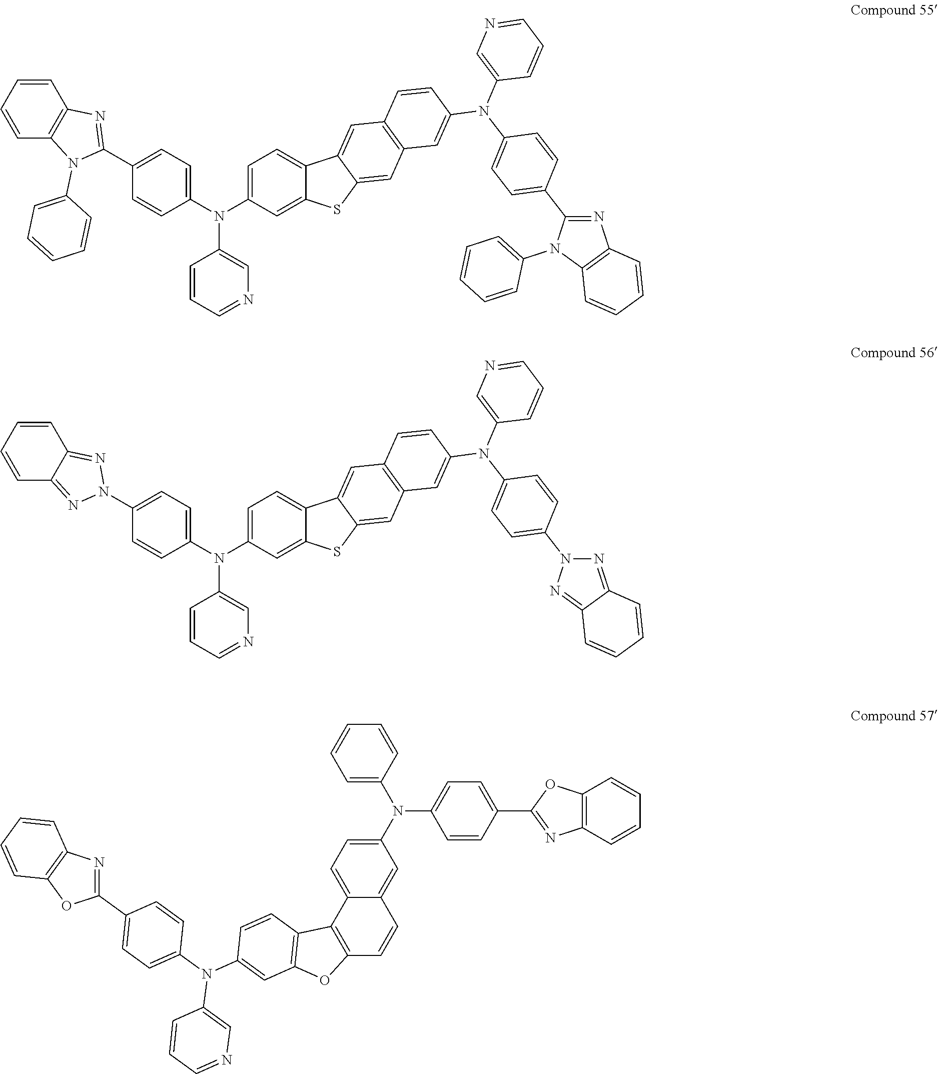 [SMILES: c1ccc(-n2c(-c3ccc(N(c4cccnc4)c4ccc5cc6c(cc5c4)sc4cc(N(c5ccc(-c7nc8ccccc8n7-c7ccccc7)cc5)c5cccnc5)ccc46)cc3)nc3ccccc32)cc1 ; c1cncc(N(c2ccc(-n3nc4ccccc4n3)cc2)c2ccc3cc4c(cc3c2)sc2cc(N(c3ccc(-n5nc6ccccc6n5)cc3)c3cccnc3)ccc24)c1 ; c1ccc(N(c2ccc(-c3nc4ccccc4o3)cc2)c2ccc3c(ccc4oc5cc(N(c6ccc(-c7nc8ccccc8o7)cc6)c6cccnc6)ccc5c43)c2)cc1]

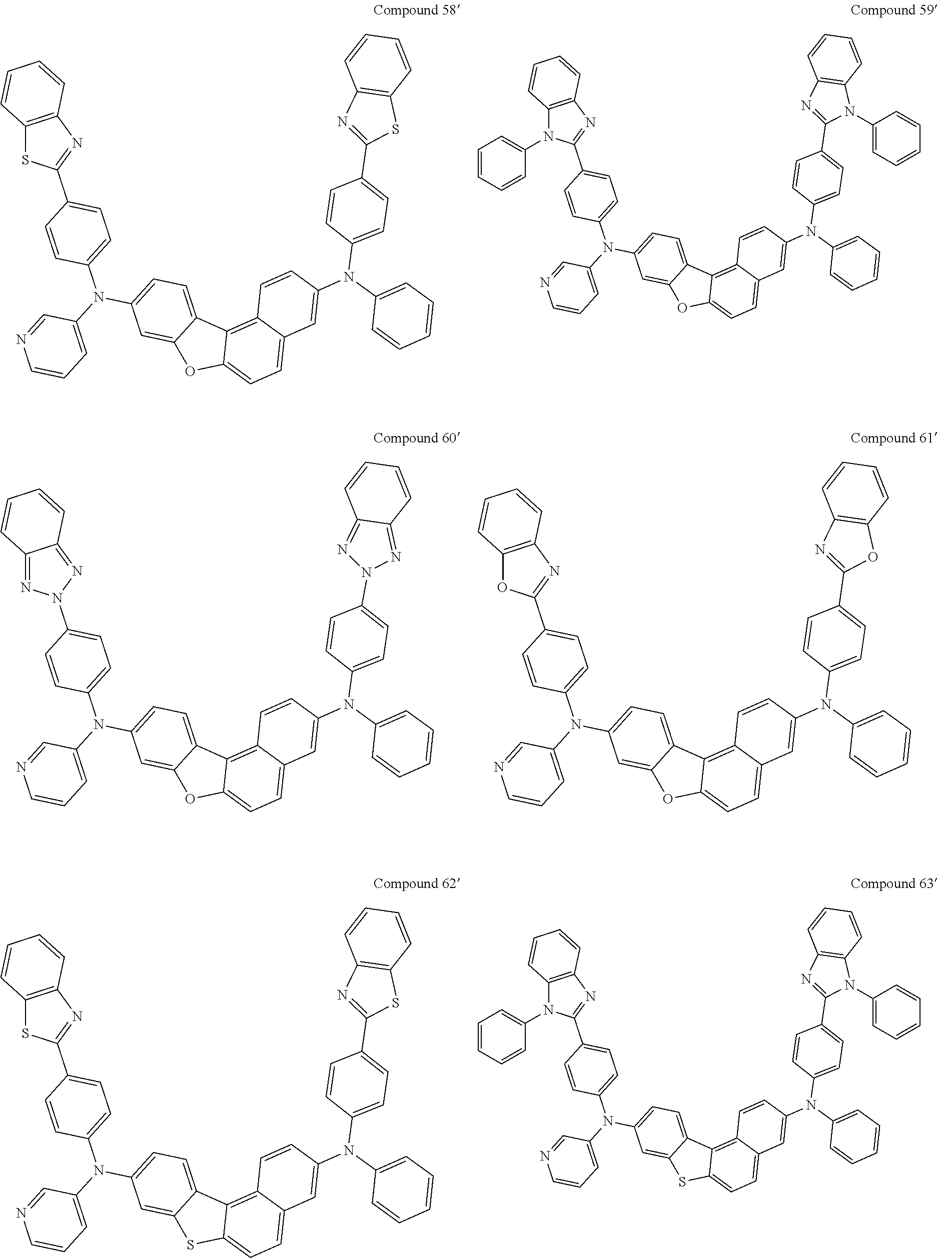

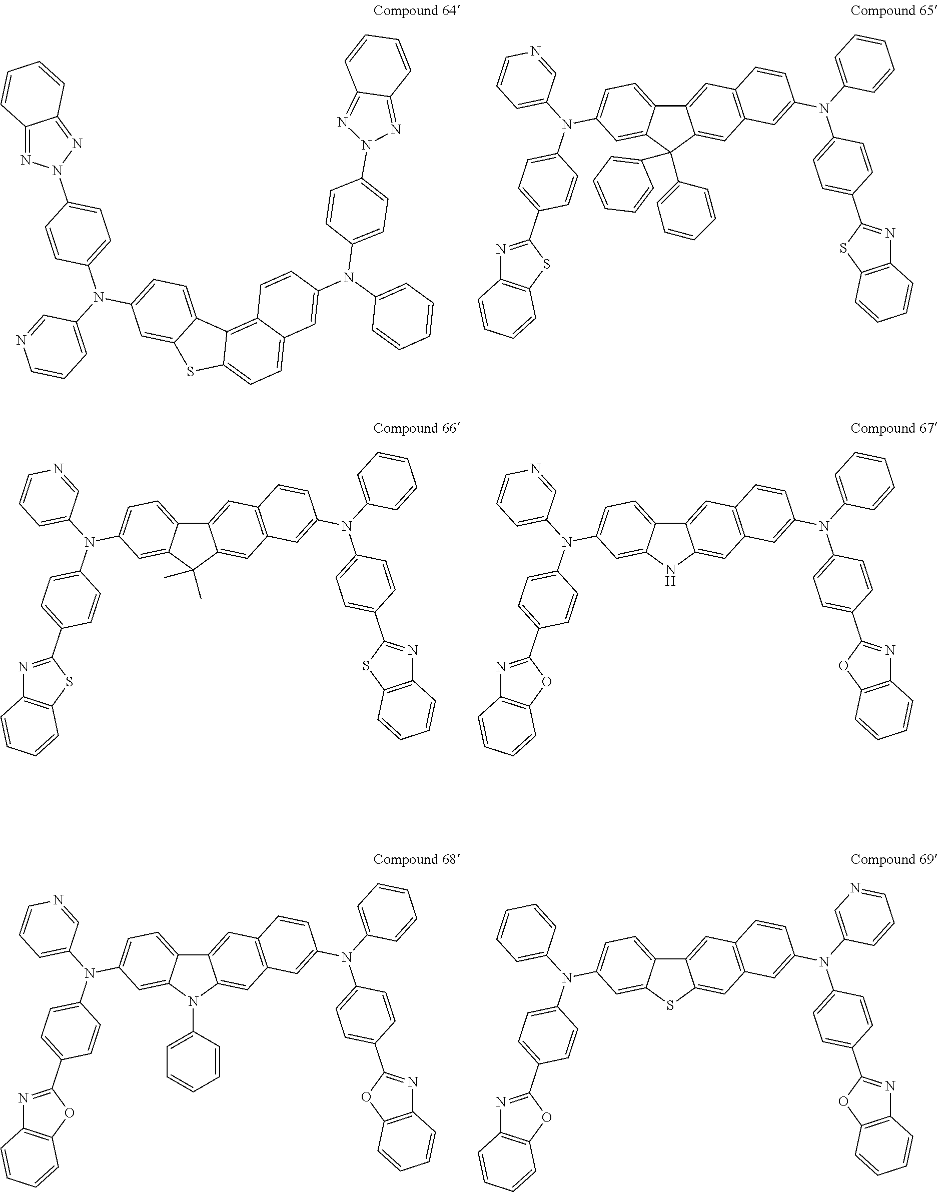

In an exemplary embodiment,
a refractive index of the diarylamine compound at a wavelength of 460 nm may be in a range of 2.08 to 2.25;
a refractive index of the aromatic amine compound at a wavelength of 530 nm may be in a range of 1.92 to 2.16; and
a refractive index of the aromatic amine compound at a wavelength of 620 nm may be in a range of 1.88 to 2.07.

In an exemplary embodiment, a light absorption coefficient of the diarylamine compound at a wavelength of 400 nm may be 0.84 or more, and the light absorption coefficient at a wavelength of 450 nm or more is 0.

In an exemplary embodiment, a glass transition temperature of the diarylamine compound may be 127° C. or above.

An embodiment of the present disclosure further provides use of the diarylamine compound described above as a light extraction material.

An embodiment of the present disclosure further provides a light extraction material, including the diarylamine compound described above.

An embodiment of the present disclosure further provides an electroluminescent device, including the diarylamine compound described above.

In an exemplary embodiment, the electroluminescent device includes a light extraction layer, and a material of the light extraction layer may include the diarylamine compound described above.

In an exemplary embodiment, the electroluminescent device may include: an anode, a Hole Injection Layer (HIL), a Hole Transport Layer (HTL), an Electron Block Layer (EBL), an Emitting Layer (EML), a Hole Block Layer (HBL), an Electron Transport Layer (ETL), an Electron Injection Layer (EIL), a cathode and a light extraction layer.

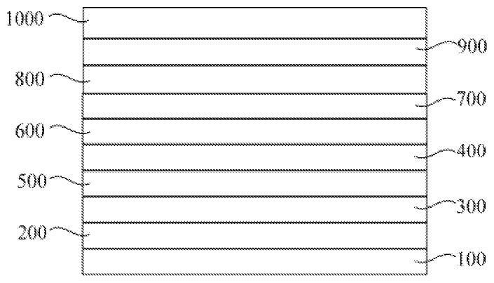

FIG. 1 is a schematic diagram of structure of an electroluminescent device according to an exemplary embodiment of the present disclosure. As shown in FIG. 1, the electroluminescent device may include: an anode 100, a hole injection layer 200, a hole transport layer 300, an electron block layer 400, an emitting layer 500, a hole block layer 600, an electron transport layer 700, an electron injection layer 800, a cathode 900 and a light extraction layer 1000. The hole injection layer 200 is disposed on a surface on one side of the anode 100, the hole transport layer 300 is disposed on a surface of the hole injection layer 200 on a side away from the anode 100, the electron block layer 400 is disposed on a surface of the hole transport layer 300 on a side away from the anode 100, the emitting layer 500 is disposed on a surface of the electron block layer 400 on a side away from the anode 100, the hole block layer 600 is disposed on a surface of the emitting layer 500 on a side away from the anode 100, the electron transport layer 700 is disposed on a surface of the hole block layer 600 on a side away from the anode 100, the electron injection layer 800 is disposed on a surface of the electron transport layer 700 on a side away from the anode 100, the cathode 900 is disposed on a surface of the electron injection layer 800 on a side away from the anode 100, and the light extraction layer 1000 is disposed on a surface of the cathode 900 on a side away from the anode 100.

In an exemplary embodiment, the light extraction layer may be formed by evaporation using a light extraction material provided by an embodiment of the present disclosure.

In an exemplary embodiment, the anode may be a material with a high work function. For example, for a bottom emission device, the anode may adopt a transparent oxide material, such as indium tin oxide (ITO) or Indium Zinc Oxide (IZO). Alternatively, for a top emission device, the anode may adopt a composite structure of a metal and a transparent oxide, such as Ag/ITO (Indium Tin Oxide), Ag/IZO (Indium Zinc Oxide), Al/ITO, Al/IZO or ITO/Ag/ITO and the like, which may ensure good reflectivity.

In an exemplary embodiment, a material of the hole injection layer may include transition metal oxides, for example, may include any one or more of molybdenum oxides, titanium oxides, vanadium oxides, rhenium oxides, ruthenium oxides, chromium oxides, zirconium oxides, hafnium oxides, tantalum oxides, silver oxides, tungsten oxides, and manganese oxides.

In another exemplary embodiment, the material of the hole injection layer may include a p-type dopant of a strong electron absorption system and a hole transport material;
the p-type dopant may include any one or more of 2,3,6,7,10,11-hexocyano-1,4,5,8,9,12-hexaazabenzophenanthrene; 2,3,5,6-tetrafluoro-7,7′,8,8′-tetracyano-p-benzoquinone (F4TCNQ); and 1,2,3-tris[(cyano)(4-cyano-2,3,5,6-tetrafluorophenyl)methylene]cyclopropane; and
the hole transport material may include any one or more of an arylamine hole transport material, a dimethylfluorene hole transport material, and a carbazole hole transport material; for example, the hole transport material may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); and 9-phenyl-3-[4-(10-phenyl-9-anthryl)phenyl]-9H-carbazole (PCzPA).

In an exemplary embodiment, the hole injection layer may be formed by evaporation.

In an exemplary embodiment, a material of the hole transport layer may include any one or more of an arylamine hole transport material, a dimethylfluorene hole transport material, and a carbazole hole transport material; for example, the material of the hole transport layer may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); and 9-phenyl-3-[4-(10-phenyl-9-anthryl)phenyl]-9H-carbazole (PCzPA).

In an exemplary embodiment, the hole transport layer may be formed by evaporation.

In an exemplary embodiment, a material of the electron block layer may include any one or more of an arylamine electron block material, a dimethylfluorene electron block material, and a carbazole electron block material; for example, the material of the electron block layer may include any one or more of 4,4′-bis[N-(1-naphthyl)-N-phenylamino]biphenyl (NPB); N,N′-bis(3-methylphenyl)-N,N′-diphenyl-[1,1′-biphenyl]-4,4′-diamine (TPD); 4-phenyl-4′-(9-phenylfluoren-9-yl)triphenylamine (BAFLP); 4,4′-bis[N-(9,9-dimethylfluoren-2-yl)-N-phenylamino]biphenyl (DFLDPBi); 4,4′-bis(9-carbazolyl)biphenyl (CBP); and 9-phenyl-3-[4-(10-phenyl-9-anthryl)phenyl]-9H-carbazole (PCzPA).

In an exemplary embodiment, the electron block layer may be formed by evaporation.

In an exemplary embodiment, a material of the emitting layer may include one luminescent material, or may include two or more luminescent materials. For example, a host luminescent material and a guest luminescent material doped into the host luminescent material may be included.

In an exemplary embodiment, the electroluminescent device may be a blue electroluminescent device, a green electroluminescent device, or a red electroluminescent device, a material of the emitting layer of the blue electroluminescent device includes a blue luminescent material, a material of the emitting layer of the green electroluminescent device includes a green luminescent material, and a material of the emitting layer of the red electroluminescent device may include a red luminescent material.

In an exemplary embodiment, the blue luminescent material may include any one or more of a pyrene derivative-based blue luminescent material, an anthracene derivative-based blue luminescent material, a fluorene derivative-based blue luminescent material, a perylene derivative-based blue luminescent material, a styrylamine derivative-based blue luminescent material, and a metal complex-based blue luminescent material.

For example, the blue luminescent material may include any one or more of N1,N6-bis([1,1′-biphenyl]-2-yl)-N1,N6-bis([1,1′-biphenyl]-4-yl)pyrene-1,6-diamine; 9,10-di-(2-naphthyl)anthracene (ADN); 2-methyl-9,10-di-2-naphthyl anthracene (MADN); 2,5,8,11-tetra-tert-butylperylene (TBPe); 4,4′-bis[4-(diphenylamino)styryl]biphenyl (BDAVBi); 4,4′-bis[4-(di-p-tolylamino)styryl]biphenyl (DPAVBi); and bis(4,6-difluorophenylpyridine-C2,N)pyridinecarboyl iridium (FIrpic).

In an exemplary embodiment, the green luminescent material may include any one or more of a coumarin dye, a quinacridone derivative green luminescent material, a polycyclic aromatic hydrocarbon green luminescent material, a diamine anthracene derivative green luminescent material, a carbazole derivative green luminescent material, and a metal complex green luminescent material.

For example, the green luminescent material may include coumarin 6(C-6); coumarin 545T (C-525T); quinacridone (QA); N,N′-dimethylquinacridone (DMQA); 5,12-diphenylnaphthonaphthalene (DPT); N10,N10′-diphenyl-N10,N10′-dibenzoyl-9,9′-dianthracene-10,10′-diamine (BA-NPB); tris(8-hydroxyquinoline) aluminum (III) (Alq3), tris(2-phenylpyridine)iridium (Ir(ppy)3); and acetylpyruvate bis(2-phenylpyridine)iridium (Ir(ppy)2(acac)).

In an exemplary embodiment, the red luminescent material may include any one or more of a DCM-based red luminescent material and a metal complex-based red luminescent material.

For example, the red luminescent material may include any one or more of 4-(dicyanomethylene)-2-methyl-6-(4-dimethylaminostyryl)-4H-pyran (DCM); 4-(dicyanomethylene)-2-tert-butyl-6-(1,1,7,7-tetramethyljulolidyl-9-enyl)-4H-pyran (DCJTB); di(1-phenylisoquinoline)(acetylacetone)iridium(III) (Ir(piq)2(acac)); octaethylporphyrin platinum (PtOEP); di(2-(2′-benzothiophenyl)pyridine-N,C3′)(acetylacetone)iridium (Ir(btp)2(acac)).

In an exemplary embodiment, the emitting layer may be formed by evaporation.

In an exemplary embodiment, the material of the hole block layer may include an aromatic heterocyclic hole block material, which, for example, may include any one or more of hole block materials based on benzimidazole and its derivatives, hole block materials based on imidazopyridine and its derivatives, hole block materials based on benzimidazophenanthridine derivatives, hole block materials based on pyrimidine and its derivatives, hole block materials based on triazine derivatives, hole block materials based on pyridine and its derivatives, hole block materials based on pyrazine and its derivatives, hole block materials based on quinoxaline and its derivatives, hole block materials based on diazole and its derivatives, hole block materials based on quinoline and its derivatives, hole block materials based on isoquinoline derivatives, hole block materials based on phenanthroline derivatives, hole block materials based on diazaphosphole, hole block materials based on phosphine oxide, hole block materials based on aromatic ketone, lactams, and hole block materials based on boranes.

For another example, the material of the hole block layer may include any one or more of 2-(4-biphenyl)-5-(4-tert-butylphenyl)-1,3,4-oxadiazole (PBD); 1,3-bis[5-(p-tert-butylphenyl)-1,3,4-oxadiazole-2-yl]benzene (OXD-7); 3-(4-tert-butylphenyl)-4-phenyl-5-(4-biphenyl)-1,2,4-triazole (TAZ); 3-(4-tert-butylphenyl)-4-(4-ethylphenyl)-5-(4-biphenyl)-1,2,4-triazole (p-EtTAZ); bathophenanthroline (BPhen); (BCP); and 4,4′-bis(5-methylbenzoxazol-2-yl)stilbene (BzOs).

In an exemplary embodiment, the hole block layer may be formed by evaporation.

In an exemplary embodiment, the material of the electron transport layer may include an aromatic heterocyclic electron transport material, which, for example, may include any one or more of electron transport materials based on benzimidazole and its derivatives, electron transport materials based on imidazopyridine and its derivatives, electron transport materials based on benzimidazophenanthridine derivatives, electron transport materials based on pyrimidine and its derivatives, electron transport materials based on triazine derivatives, electron transport materials based on pyridine and its derivatives, electron transport materials based on pyrazine and its derivatives, electron transport materials based on quinoxaline and its derivatives, electron transport materials based on diazole and its derivatives, electron transport materials based on quinoline and its derivatives, electron transport materials based on isoquinoline derivatives, electron transport materials based on phenanthroline derivatives, electron transport materials based on diazaphosphole, electron transport materials based on phosphine oxide, electron transport materials based on aromatic ketone, lactams, and electron transport materials based on boranes.

For another example, the material of the electron transport layer may include any one or more of 2-(4-biphenyl)-5-(4-tert-butylphenyl)-1,3,4-oxadiazole (PBD); 1,3-bis[5-(p-tert-butylphenyl)-1,3,4-oxadiazole-2-yl]benzene (OXD-7); 3-(4-tert-butylphenyl)-4-phenyl-5-(4-biphenyl)-1,2,4-triazole (TAZ); 3-(4-tert-butylphenyl)-4-(4-ethylphenyl)-5-(4-biphenyl)-1,2,4-triazole (p-EtTAZ); bathophenanthroline (BPhen); (BCP); and 4,4′-bis(5-methylbenzoxazol-2-yl)stilbene (BzOs).

In an exemplary embodiment, the electron transport layer may be formed by evaporation.

In an exemplary embodiment, the material of the electron injection layer may include any one or more of an alkali metal electron injection material and a metal electron injection material.

For example, the material of the electron injection layer may include any one or more of LiF, Yb, Mg, and Ca.

In an exemplary embodiment, the electron injection layer may be formed by evaporation.

In an exemplary embodiment, the cathode may be formed by a metal with a relatively low work function, such as Al, Ag, and Mg, or formed by an alloy containing a metal material with a low work function.

An embodiment of the present disclosure further provides a display apparatus, including the electroluminescent device described above.

In an exemplary embodiment, the display apparatus may include a plurality of electroluminescent devices. For example, the electroluminescent device may be a blue electroluminescent device, a green electroluminescent device, or a red electroluminescent device, and the display apparatus may include a blue electroluminescent device, a green electroluminescent device, and a red electroluminescent device.

The display apparatus may be any product or component with a display function, such as a mobile phone, a tablet computer, a television, a display, a laptop computer, a digital photo frame, a navigator, a vehicle-mounted display, a smart watch, and a smart bracelet.

Below are synthesis processes and performance tests and comparisons of diarylamine compounds according to some exemplary embodiments of the present disclosure.

The exemplary synthesis methods of diarylamine compounds of the present disclosure are as follows.

### Synthesis Example 1: Synthesis of Compound 1

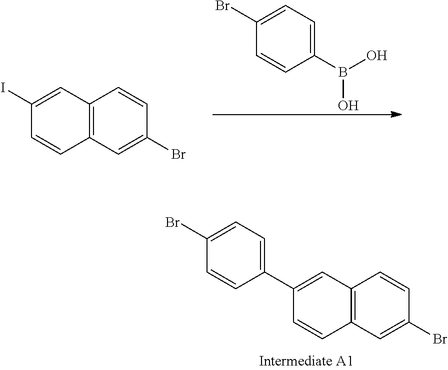 [SMILES: Brc1ccc2cc(I)ccc2c1 ; OB(O)c1ccc(Br)cc1 ; Brc1ccc(-c2ccc3cc(Br)ccc3c2)cc1]

### Synthesis of Intermediate A1

0.3 mol of 2-bromo-6-iodonaphthalene and 0.3 mol of p-bromophenylboronic acid were added to a reaction flask, then 500 ml of toluene, ethanol and an aqueous solution of potassium carbonate were added; vacuumizing was performed and nitrogen was filled, 0.003 mol of catalyst tetra(triphenylphosphine)palladium was added, the vacuumizing and the filling of nitrogen were continued, the reaction was performed by heating, refluxing and stirring for 4 hours, then water was added and the mixture was stirred, cooled to room temperature, filtered under reduced pressure, and rinsed with hot water and acetone sequentially to ensure that the pH value of a filtrate was about 7; chloroform was added for dissolving, the filtrate was concentrated and then a small amount of methanol was added for recrystallizing, and the mixture was filtered under reduced pressure to obtain a solid, i.e. intermediate A1, with a yield of about 88%.

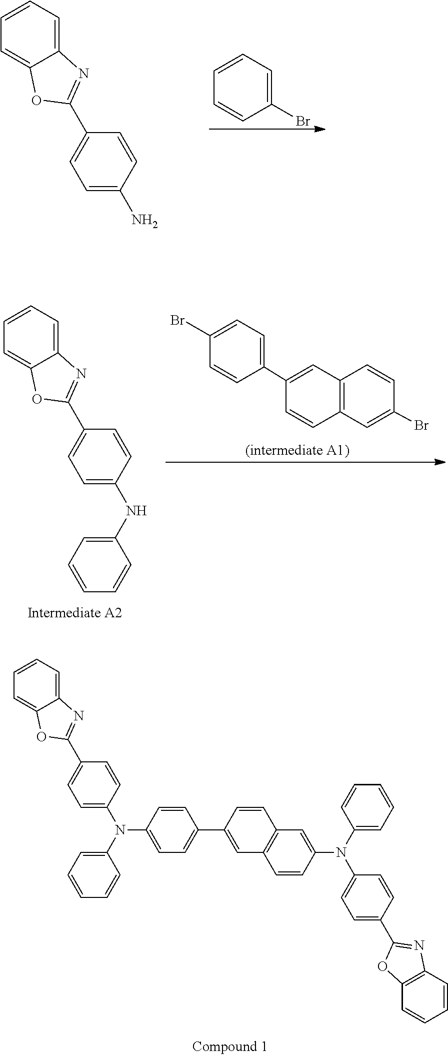 [SMILES: Nc1ccc(-c2nc3ccccc3o2)cc1 ; Brc1ccccc1 ; c1ccc(Nc2ccc(-c3nc4ccccc4o3)cc2)cc1 ; Brc1ccc(-c2ccc3cc(Br)ccc3c2)cc1 ; c1ccc(N(c2ccc(-c3ccc4cc(N(c5ccccc5)c5ccc(-c6nc7ccccc7o6)cc5)ccc4c3)cc2)c2ccc(-c3nc4ccccc4o3)cc2)cc1]

### Synthesis of Intermediate A2

400 ml of a toluene solvent was added to a reaction flask, then 0.02 mol of 4-(benzo[D]oxazol-2-yl)phenylamine, 0.2 mol of bromobenzene and 0.2 mol of sodium tert-butoxide were added; nitrogen was filled and then 0.002 mol of palladium acetate was added; nitrogen was refilled, 0.007 mol of a toluene solution of tri-tert-butyl phosphine was added; nitrogen was refilled and then the reaction was refluxed for 2 hours; after the reaction was finished, the mixture was cooled to room temperature, filtered through diatomite to obtain a filtrate, the filtrate was concentrated, then methanol was added, the mixture was left to stand for recrystallization, followed by suction filtration and rinsing with methanol to obtain a recrystallized solid, thereby intermediate A2, with a yield of 75%, was obtained;

### Synthesis of Compound 1

100 ml of a toluene solvent was added to a reaction flask, then 0.04 mol of raw material intermediate A2, 0.02 mol of intermediate A1 and 0.07 mol of sodium tert-butoxide were sequentially added; nitrogen was filled and then 0.1 g of palladium acetate was added; nitrogen was refilled, tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated and then heated, a small amount of ethanol was added, the mixture was left to stand to room temperature to recrystallize, followed by suction filtration and rinsing with ethanol to obtain a recrystallized solid, thereby a light yellow solid, i.e. compound 1, with a yield of 72%, was obtained.

Mass spectrum m/z: 772.28, element content (%): C₅₄H₃₆N₄O₂, C, 83.92; H, 4.69; O, 4.14; N, 7.25.

¹H NMR (500 MHz, CDCl₃): δ 7.84 (1H), 7.74-7.73 (8H), 7.56-7.55 (3H), 7.37-7.4 (11H), 7.32-7.24 (5H), 7.17-7.00 (8H).

### Synthesis Example 2: Synthesis of Compound 2

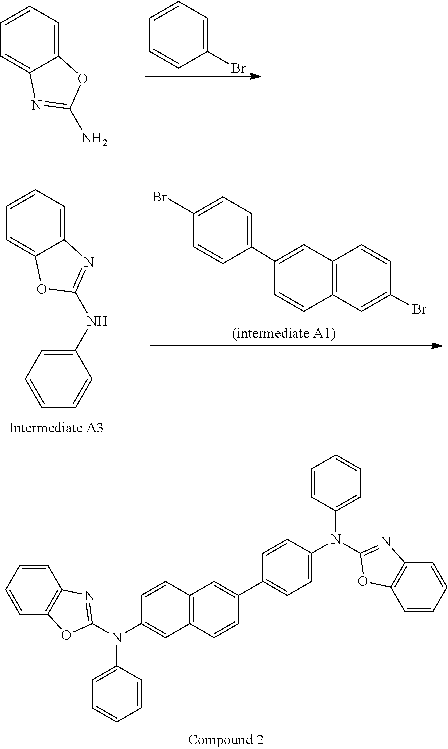 [SMILES: Nc1nc2ccccc2o1 ; Brc1ccccc1 ; c1ccc(Nc2nc3ccccc3o2)cc1 ; Brc1ccc(-c2ccc3cc(Br)ccc3c2)cc1 ; c1ccc(N(c2ccc(-c3ccc4cc(N(c5ccccc5)c5nc6ccccc6o5)ccc4c3)cc2)c2nc3ccccc3o2)cc1]

The synthesis process of intermediate A1 was the same as that in synthesis example 1.

The synthesis process of intermediate A3 was similar to that of intermediate A2 in synthesis example 1, except that the raw material 4-(benzo[D]oxazol-2-yl)phenylamine was replaced with 2-amino-benzoxazole, with a yield of 83%.

The synthesis process of compound 2 was similar to that of compound 1 in synthesis example 1, except that intermediate A2 was replaced with intermediate A3, with a yield of 76%.

Mass spectrum m/z: 620.22, element content (%): C₄₂H₂₈N₄O₂, C, 81.27; H, 4.55; 0, 5.16; N, 9.03.

¹H NMR (500 MHz, CDCl₃): δ 7.84 (1H), 7.72 (4H), 7.56-7.55 (4H), 7.4-7.39 (5H), 7.37 (4H), 7.32 (1H), 7.24 (4H), 7.17-7.08 (6H), 7.00 (2H).

### Synthesis Example 3: Synthesis of Compound 3

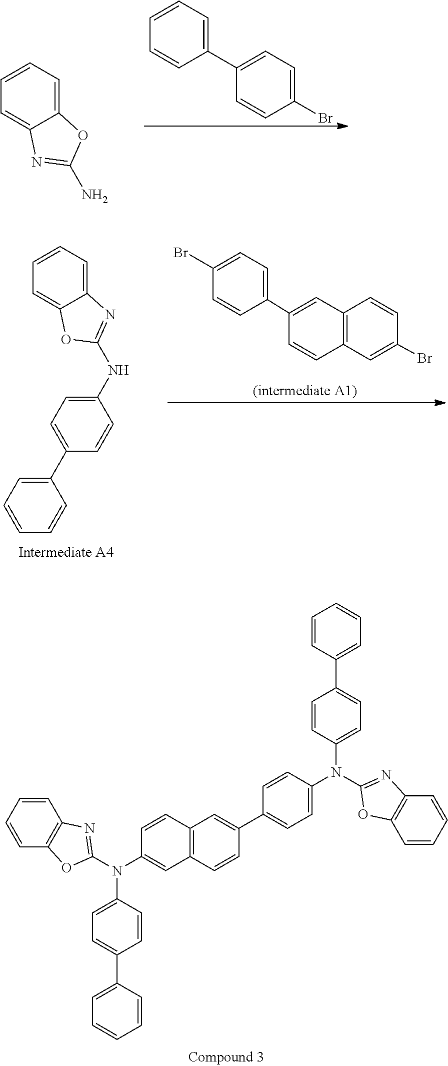 [SMILES: Nc1nc2ccccc2o1 ; Brc1ccc(-c2ccccc2)cc1 ; c1ccc(-c2ccc(Nc3nc4ccccc4o3)cc2)cc1 ; Brc1ccc(-c2ccc3cc(Br)ccc3c2)cc1 ; c1ccc(-c2ccc(N(c3ccc(-c4ccc5cc(N(c6ccc(-c7ccccc7)cc6)c6nc7ccccc7o6)ccc5c4)cc3)c3nc4ccccc4o3)cc2)cc1]

The synthesis process of intermediate A4 was similar to that of intermediate A3 in synthesis example 2, except that the raw material bromobenzene was replaced with 4-bromobiphenyl, with a yield of 73%.

The synthesis process of compound 3 was similar to that of compound 2 in synthesis example 2, except that intermediate A3 was replaced with intermediate A4, with a yield of 75%.

Mass spectrum m/z: 772.28, element content (%): C₅₄H₃₆N₄O₂, C, 83.92; H, 4.69; 0, 4.14; N, 7.25.

¹H NMR (500 MHz, CDCl₃): δ 7.84 (1H), 7.75 (4H), 7.72 (4H), 7.56-7.55 (7H), 7.49 (4H), 7.41-7.4 (3H), 7.39 (4H), 7.37 (6H), 7.32 (1H), 7.17-7.11 (2H).

### Synthesis Example 4: Synthesis of Compound 5

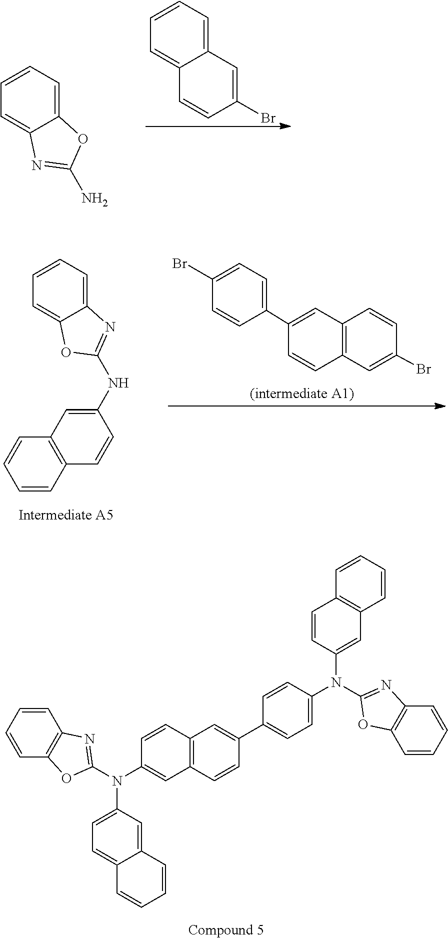 [SMILES: Nc1nc2ccccc2o1 ; Brc1ccc2ccccc2c1 ; c1ccc2cc(Nc3nc4ccccc4o3)ccc2c1 ; Brc1ccc(-c2ccc3cc(Br)ccc3c2)cc1 ; c1ccc2cc(N(c3ccc(-c4ccc5cc(N(c6ccc7ccccc7c6)c6nc7ccccc7o6)ccc5c4)cc3)c3nc4ccccc4o3)ccc2c1]

The synthesis process of intermediate E was similar to that of intermediate A3 in synthesis example 2, except that the raw material bromobenzene was replaced with 2-bromonaphthalene, with a yield of 88%.

The synthesis process of compound 5 was similar to that of compound 2 in synthesis example 2, except that intermediate A3 was replaced with intermediate E, with a yield of 70%.

Mass spectrum m/z: 720.25, element content (%): C₅₀H₃₂N₄O₂, C, 83.31; H, 4.47; 0, 4.44; N, 7.77.

¹H NMR (500 MHz, CDCl₃): δ 7.84 (1H), 7.78 (2H), 7.72 (4H), 7.71 (2H), 7.56 (1H), 7.55 (2H), 7.54 (2H), 7.45 (2H), 7.42 (2H), 7.4 (3H), 7.39 (4H), 7.37 (2H), 7.32 (1H), 7.17-7.11 (4H).

### Synthesis Example 5: Synthesis of Compound 7

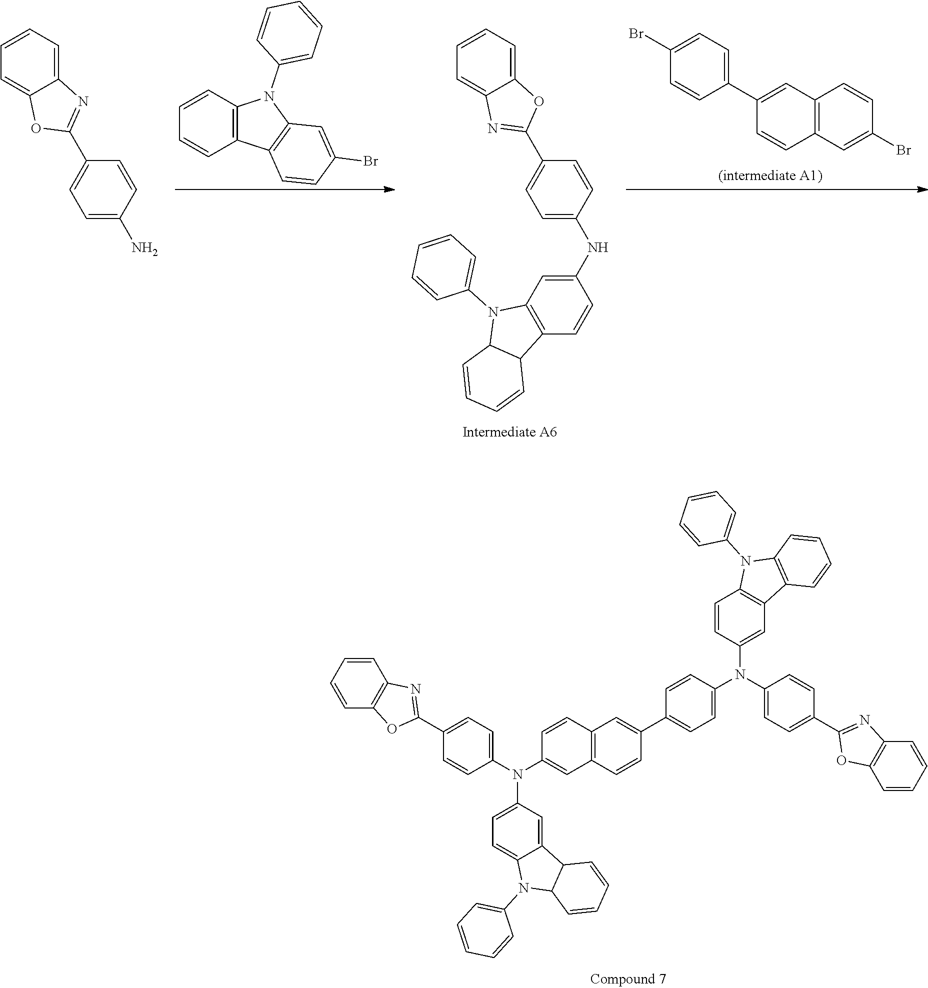 [SMILES: Nc1ccc(-c2nc3ccccc3o2)cc1 ; Brc1ccc2c3ccccc3n(-c3ccccc3)c2c1 ; C1=CC2c3ccc(Nc4ccc(-c5nc6ccccc6o5)cc4)cc3N(c3ccccc3)C2C=C1 ; Brc1ccc(-c2ccc3cc(Br)ccc3c2)cc1 ; C1=CC2c3cc(N(c4ccc(-c5nc6ccccc6o5)cc4)c4ccc5cc(-c6ccc(N(c7ccc(-c8nc9ccccc9o8)cc7)c7ccc8c(c7)c7ccccc7n8-c7ccccc7)cc6)ccc5c4)ccc3N(c3ccccc3)C2C=C1]

The synthesis process of intermediate A6 was similar to that of intermediate A2 in synthesis example 1, except that the raw material bromobenzene was replaced with 2-bromo(9-phenyl)carbazole, with a yield of 71%.

The process of compound 7 was similar to the synthesis process of compound 1 in synthesis example 1, except that intermediate A2 was replaced with intermediate A6, with a yield of 68%.

Mass spectrum m/z: 1102.4, element content (%): C₇₈H₅₀N₆O₂, C, 84.91; H, 4.57; 0, 2.90; N, 7.62

¹H NMR (500 MHz, CDCl₃): δ 8.55 (2H), 7.94 (2H), 7.84 (1H), 7.74 (4H), 7.73 (4H), 7.62 (4H), 7.58 (2H), 7.56 (1H), 7.55 (2H), 7.54 (2H), 7.5 (4H), 7.4-7.38 (5H), 7.37 (6H), 7.35 (4H), 7.33 (2H), 7.32 (1H), 7.17-7.11 (4H).

### Synthesis Example 6: Synthesis of Compound 12

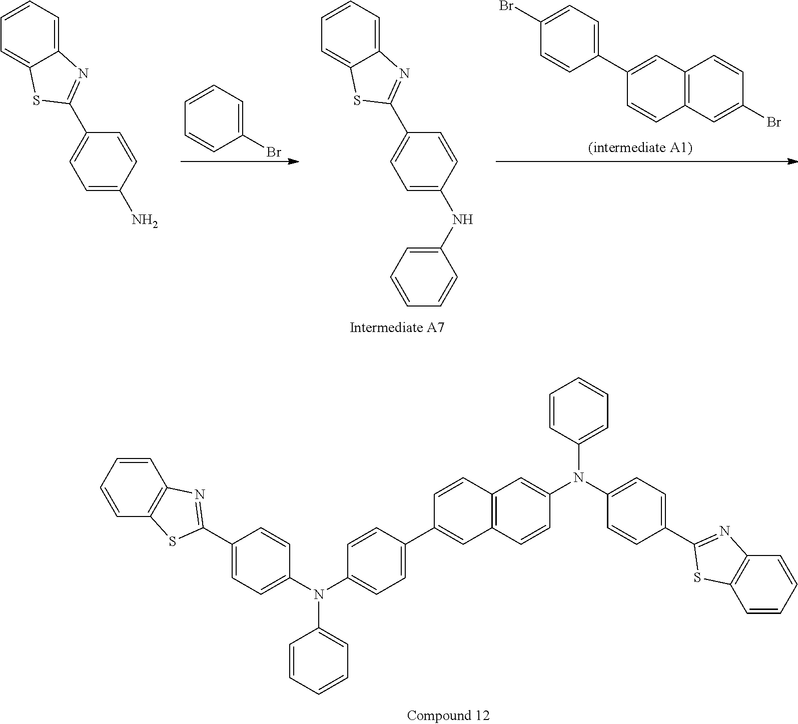 [SMILES: Nc1ccc(-c2nc3ccccc3s2)cc1 ; Brc1ccccc1 ; c1ccc(Nc2ccc(-c3nc4ccccc4s3)cc2)cc1 ; Brc1ccc(-c2ccc3cc(Br)ccc3c2)cc1 ; c1ccc(N(c2ccc(-c3ccc4cc(N(c5ccccc5)c5ccc(-c6nc7ccccc7s6)cc5)ccc4c3)cc2)c2ccc(-c3nc4ccccc4s3)cc2)cc1]

The synthesis process of intermediate A7 was similar to that of intermediate A2 in synthesis example 1, except that the raw material 4-(benzo[D]oxazol-2-yl)phenylamine was replaced with 4-(2-benzothiazolyl)phenylamine, with a yield of 80%.

The synthesis process of compound 12 was similar to that of compound 1 in synthesis example 1, except that intermediate A2 was replaced with intermediate A7, with a yield of 74%.

Mass spectrum m/z: 804.24, element content (%): C₅₄H₃₆N₄S₂, C, 80.57; H, 4.51; S, 7.96; N, 6.96

¹H NMR (500 MHz, CDCl₃): δ 8.18 (2H), 8.02 (2H), 7.85-7.84 (5H), 7.56-7.55 (3H), 7.53 (2H), 7.51 (2H), 7.4 (1H), 7.37 (6H), 7.32 (1H), 7.24 (4H), 7.17 (1H), 7.11 (1H), 7.08 (4H), 7.00 (2H).

### Synthesis Example 7: Synthesis of Compound 16

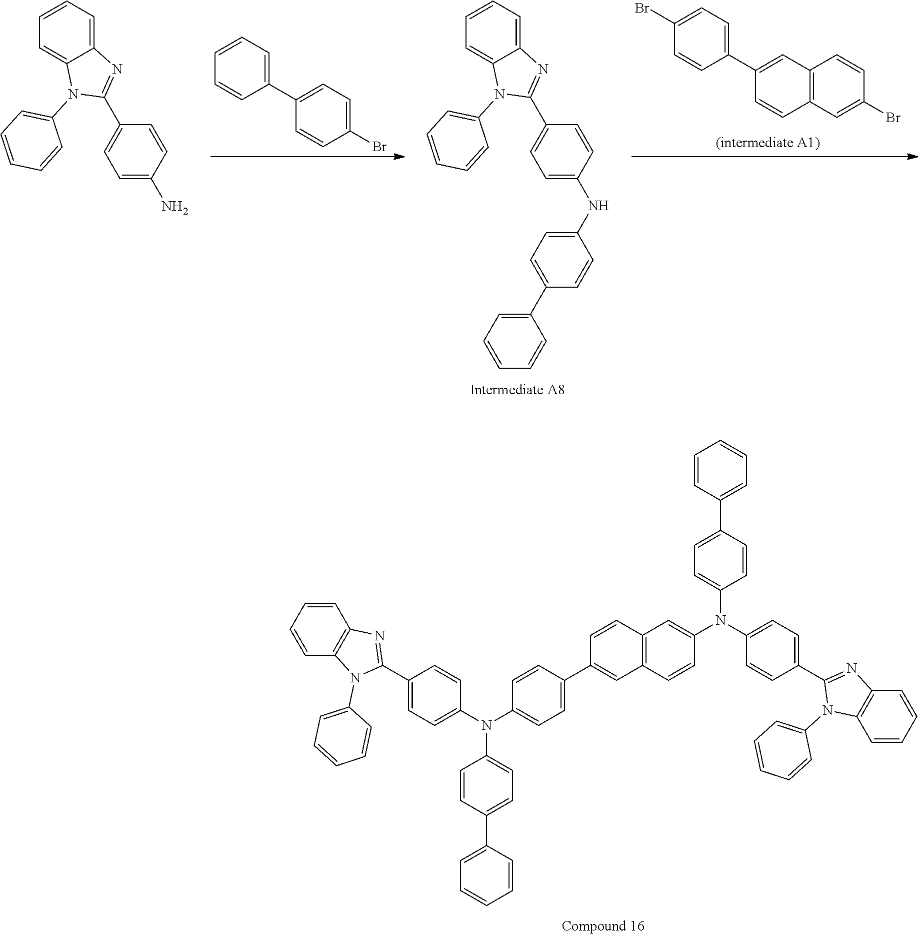 [SMILES: Nc1ccc(-c2nc3ccccc3n2-c2ccccc2)cc1 ; Brc1ccc(-c2ccccc2)cc1 ; c1ccc(-c2ccc(Nc3ccc(-c4nc5ccccc5n4-c4ccccc4)cc3)cc2)cc1 ; Brc1ccc(-c2ccc3cc(Br)ccc3c2)cc1 ; c1ccc(-c2ccc(N(c3ccc(-c4ccc5cc(N(c6ccc(-c7ccccc7)cc6)c6ccc(-c7nc8ccccc8n7-c7ccccc7)cc6)ccc5c4)cc3)c3ccc(-c4nc5ccccc5n4-c4ccccc4)cc3)cc2)cc1]

The synthesis process of intermediate A8 was similar to that of intermediate A7 in synthesis example 6, except that the raw material 4-(2-benzothiazolyl)phenylamine was replaced with 4-(1-phenylbenzimidazol-2-yl)phenylamine and bromobenzene was replaced with 4-bromobiphenyl, with a yield of 65%.

The synthesis process of compound 16 was similar to that of compound 12 in synthesis example 6, except that intermediate A7 was replaced with intermediate A8, with a yield of 62%.

Mass spectrum m/z: 1074.44, element content (%): C₇₈H₅₄N₆, C, 87.12; H, 5.06; N, 7.82 ¹H NMR (500 MHz, CDCl₃): δ 8.56 (2H), 8.01 (4H), 7.84-7.81 (3H), 7.75 (4H), 7.62 (2H), 7.56-7.55 (7H), 7.53 (2H), 7.49 (4H), 7.48 (4H), 7.41-7.4 (3H), 7.38 (4H), 7.37 (10H), 7.32 (1H), 7.28 (2H), 7.17-7.11 (2H).

### Synthesis Example 8: Synthesis of Compound 11′

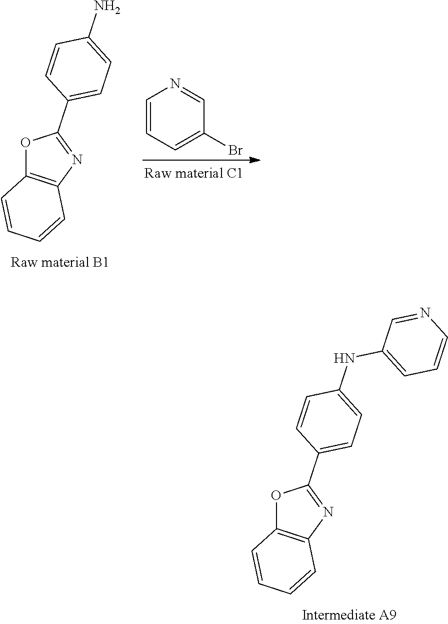

400 mL of a toluene solvent was added to a reaction flask, then 0.2 mol of raw material B1, 0.2 mol of raw material C1 and 0.1 mol of sodium tert-butoxide were added, nitrogen was filled and then 0.002 mol of palladium acetate was added, nitrogen was refilled, 0.007 mol of a toluene solution of tri-tert-butyl phosphine was added; nitrogen was refilled, the reaction was refluxed for 2 hours; after the reaction was finished, the mixture was cooled to room temperature, filtered through diatomite to obtain a filtrate, the filtrate was concentrated, then methanol was added, and the mixture was left to stand for recrystallization, followed by suction filtration and rinsing with methanol to obtain a recrystallized solid, thereby intermediate A9, with a yield of 78%, was obtained.

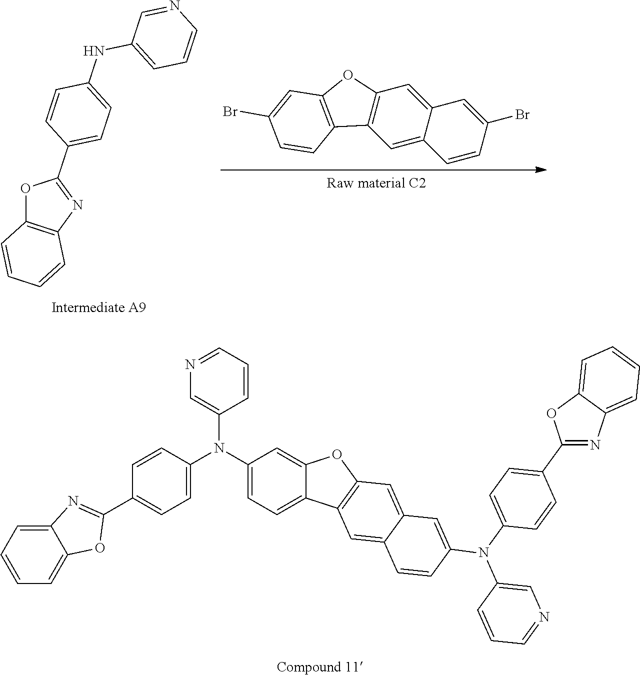 [SMILES: c1cncc(Nc2ccc(-c3nc4ccccc4o3)cc2)c1 ; Brc1ccc2cc3c(cc2c1)oc1cc(Br)ccc13 ; c1cncc(N(c2ccc(-c3nc4ccccc4o3)cc2)c2ccc3cc4c(cc3c2)oc2cc(N(c3ccc(-c5nc6ccccc6o5)cc3)c3cccnc3)ccc24)c1]

400 ml of a toluene solvent was added to a reaction flask, and then 0.3 mol of intermediate A9, 0.15 mol of raw material C2 and 0.1 mol of sodium tert-butoxide were sequentially added; nitrogen was filled and then 0.002 mol of palladium acetate was added, nitrogen was refilled, 0.007 mol of a toluene solution of tri-tert-butyl phosphine was added; nitrogen was refilled, the reaction was refluxed for 2 hours; after the reaction was finished, the mixture was cooled to room temperature, filtered through diatomite to obtain a filtrate, the filtrate was concentrated and then heated, a small amount of ethanol was added, and the mixture was left to stand to room temperature to recrystallize, followed by suction filtration and rinsing with ethanol to obtain a recrystallized solid, thereby a solid compound 11′, with a yield of 75%, was obtained.

Mass spectrum m/z: 788.25, element content (%): C₅₂H₃₂N₆O₃, C, 79.17; H, 4.09; O, 6.08; N, 10.65.

¹H NMR (500 MHz, CDCl₃): δ 8.03-8.04 (3H), 7.92 (2H), 7.8 (1H), 7.73-7.74 (8H), 7.54 (1H), 7.49 (1H), 7.42 (2H), 7.36-7.38 (10H), 7.11-7.11 (3H), 6.91 (1H).

### Synthesis Example 9: Synthesis of Compound 12′

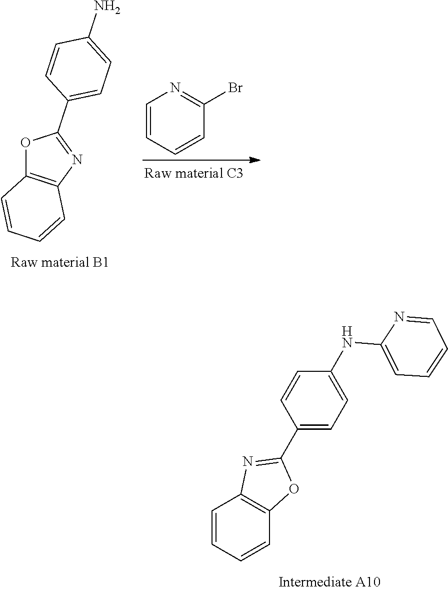 [SMILES: Nc1ccc(-c2nc3ccccc3o2)cc1 ; Brc1ccccn1 ; c1ccc(Nc2ccc(-c3nc4ccccc4o3)cc2)nc1]

400 ml of a toluene solvent was added to a reaction flask, 0.2 mol of raw material B1, 0.2 mol of raw material C3 and 0.1 mol of sodium tert-butoxide were added, nitrogen was filled, then 0.002 mol of palladium acetate was added, nitrogen was refilled, 0.007 mol of a toluene solution of tri-tert-butyl phosphine was added; nitrogen was refilled, the reaction was refluxed for 2 hours; after the reaction was finished, the mixture was cooled to room temperature, filtered through diatomite to obtain a filtrate, the filtrate was concentrated, then methanol was added, and the mixture was left to stand for recrystallization, followed by suction filtration and rinsing with methanol to obtain a recrystallized solid, thereby intermediate A10, with a yield of 76%, was obtained.

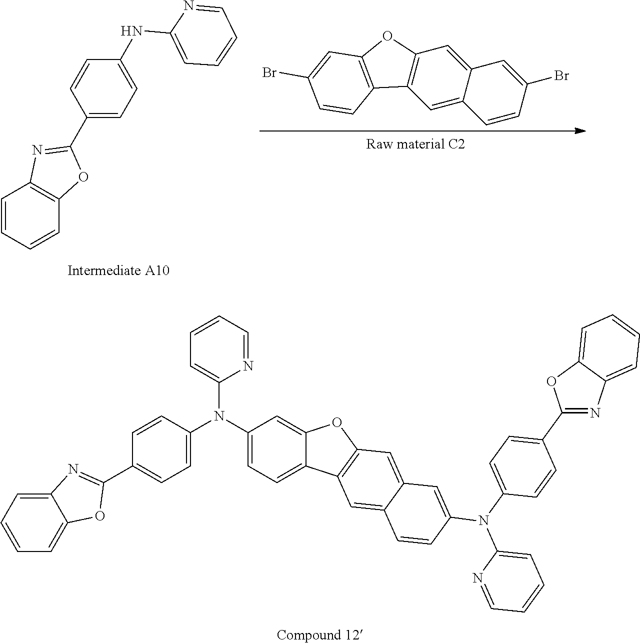 [SMILES: c1ccc(Nc2ccc(-c3nc4ccccc4o3)cc2)nc1 ; Brc1ccc2cc3c(cc2c1)oc1cc(Br)ccc13 ; c1ccc(N(c2ccc(-c3nc4ccccc4o3)cc2)c2ccc3cc4c(cc3c2)oc2cc(N(c3ccc(-c5nc6ccccc6o5)cc3)c3ccccn3)ccc24)nc1]

400 ml of a toluene solvent was added to a reaction flask, and then 0.3 mol of intermediate A10, 0.15 mol of raw material C2 and 0.1 mol of sodium tert-butoxide were sequentially added; nitrogen was filled and then 0.002 mol of palladium acetate was added, nitrogen was refilled, 0.007 mol of a toluene solution of tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated and then heated, a small amount of ethanol was added, and the mixture was left to stand to room temperature for recrystallization, followed by suction filtration and rinsing with ethanol to obtain a recrystallized solid, thereby a solid compound A12′, with a yield of 70%, was obtained.

Mass spectrum m/z: 788.2536: C₅₂H₃₂N₆O₃, C, 79.17; H, 4.09; N, 10.65; O, 6.08.

¹H NMR (500 MHz, CDCl₃): δ 8.03-8.04 (3H), 7.8 (1H), 7.73-7.74 (8H), 7.54-7.55 (3H), 7.49 (1H), 7.42 (2H), 7.38-7.37 (8H), 7.11 (1H), 6.91 (1H), 6.73 (2H), 6.63 (2H).

### Synthesis Example 10: Synthesis of Compound 13′

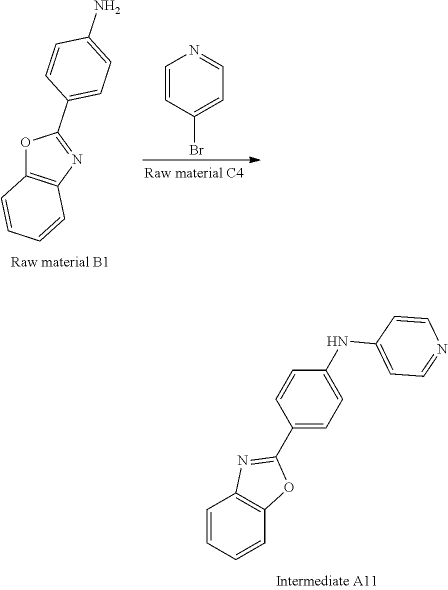 [SMILES: Nc1ccc(-c2nc3ccccc3o2)cc1 ; Brc1ccncc1 ; c1ccc2oc(-c3ccc(Nc4ccncc4)cc3)nc2c1]

400 ml of a toluene solvent was added to a reaction flask, then 0.2 mol of raw material B1, 0.2 mol of raw material C4 and 0.1 mol of sodium tert-butoxide were added, nitrogen was filled, 0.002 mol of palladium acetate was added, nitrogen was refilled, 0.007 mol of a toluene solution of tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated, then methanol was added, and the mixture was left to stand for recrystallization, followed by suction filtration and rinsing with methanol to obtain a recrystallized solid, thereby intermediate A11, with a yield of 7800, was obtained.

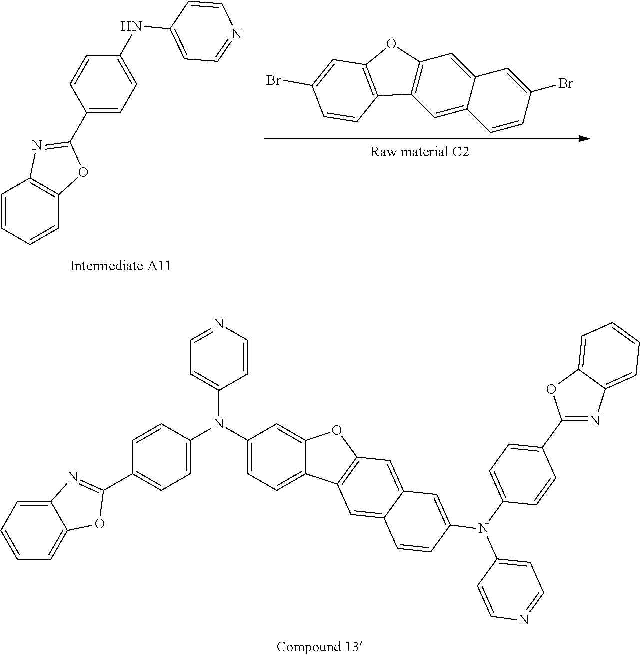 [SMILES: c1ccc2oc(-c3ccc(Nc4ccncc4)cc3)nc2c1 ; Brc1ccc2cc3c(cc2c1)oc1cc(Br)ccc13 ; c1ccc2oc(-c3ccc(N(c4ccncc4)c4ccc5cc6c(cc5c4)oc4cc(N(c5ccncc5)c5ccc(-c7nc8ccccc8o7)cc5)ccc46)cc3)nc2c1]

400 ml of a toluene solvent was added to a reaction flask, then 0.3 mol of intermediate A11, 0.15 mol of raw material C2 and 0.1 mol of sodium tert-butoxide were sequentially added; nitrogen was filled and then 0.002 mol of palladium acetate was added, nitrogen was refilled, 0.007 mol of a toluene solution of tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated and then heated, a small amount of ethanol was added, and the mixture was left to stand to room temperature to recrystallize, followed by suction filtration and rinsing with ethanol to obtain a recrystallized solid, thereby a solid compound 13′, with a yield of 76%, was obtained.

Mass spectrum m/z: 788.2536: C₅₂H₃₂N₆O₃, C, 79.17; H, 4.09; N, 10.65; 0, 6.08;

¹H NMR (500 MHz, CDCl₃): δ 8.46 (4H), 8.03 (1H), 7.8 (1), 7.73-7.74 (8H), 7.5 (1H), 7.49 (1H), 7.42 (2H), 7.38-7.37 (8H), 7.11 (1H), 6.99 (4H), 6.91 (1H).

### Synthesis Example 11: Synthesis of Compound 32′

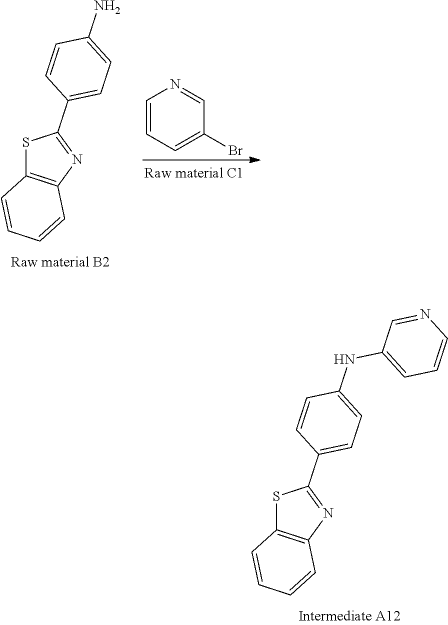

400 ml of a toluene solvent was added to a reaction flask, then 0.2 mol of raw material B2, 0.2 mol of raw material C1 and 0.1 mol of sodium tert-butoxide were added, nitrogen was filled and then 0.002 mol of palladium acetate was added, nitrogen was refilled, 0.007 mol of a toluene solution of tri-tert-butyl phosphine was added; nitrogen was refilled and then the reaction was refluxed for 2 hours; after the reaction was finished, the mixture was cooled to room temperature, filtered through diatomite to obtain a filtrate, the filtrate was concentrated, then methanol was added, and the mixture was left to stand for recrystallization, followed by suction filtration and rinsing with methanol to obtain a recrystallized solid, thereby intermediate A12, with a yield of 71%, was obtained.

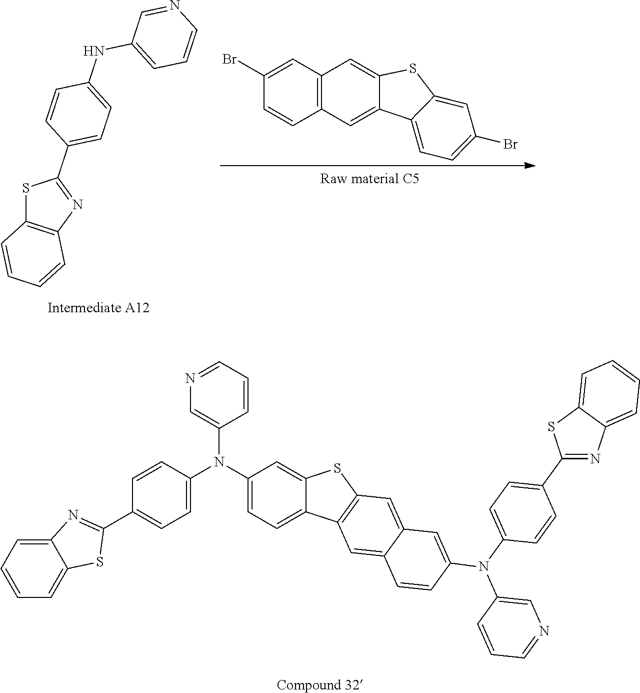 [SMILES: c1cncc(Nc2ccc(-c3nc4ccccc4s3)cc2)c1 ; Brc1ccc2cc3c(cc2c1)sc1cc(Br)ccc13 ; c1cncc(N(c2ccc(-c3nc4ccccc4s3)cc2)c2ccc3cc4c(cc3c2)sc2cc(N(c3ccc(-c5nc6ccccc6s5)cc3)c3cccnc3)ccc24)c1]

The synthesis process of compound 32′ was similar to that of compound 11′ in synthesis example 8, except that the raw materials were replaced with intermediate A12 and raw material C5, with a yield of 75%.

Mass spectrum m/z: 836.19, element content (%): C₅₂H₃₂N₆S₃, C, 74.62; H, 3.85; N, 10.04; S, 11.49.

¹H NMR (500 MHz, CDCl₃): δ 8.18 (2H), 8.01-8.04 (5H), 7.92 (2H), 7.85-7.86 (5H), 7.73-7.74 (8H), 7.78 (1H), 7.64 (1H), 7.54 (1H), 7.51-7.53 (3H), 7.42-7.43 (2H), 7.36-7.37 (6H), 7.24 (4), 7.11-7.15 (3H).

### Synthesis Example 12: Synthesis of Compound 41′

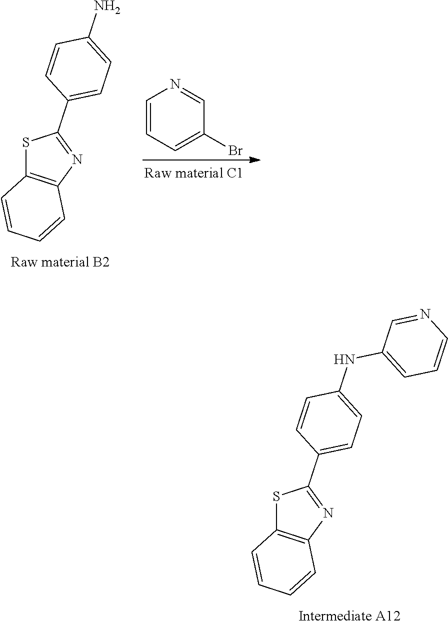

400 ml of a toluene solvent was added to a reaction flask, then 0.2 mol of raw material B2, 0.2 mol of raw material C1 and 0.1 mol of sodium tert-butoxide were added, nitrogen was filled and then 0.002 mol of palladium acetate was added, nitrogen was refilled, 0.007 mol of a toluene solution of tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated, then methanol was added, and the mixture was left to stand for recrystallization, followed by suction filtration and rinsing with methanol to obtain a recrystallized solid, thereby obtaining intermediate A12, with a yield of 77%.

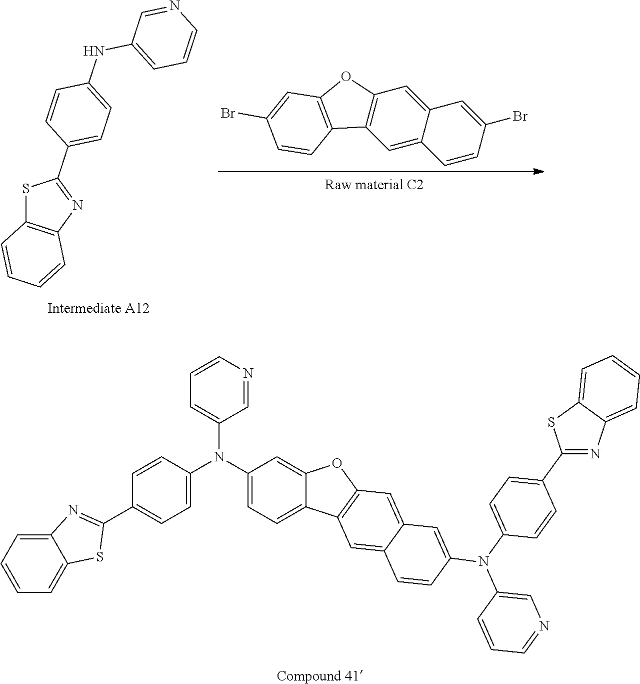 [SMILES: c1cncc(Nc2ccc(-c3nc4ccccc4s3)cc2)c1 ; Brc1ccc2cc3c(cc2c1)oc1cc(Br)ccc13 ; c1cncc(N(c2ccc(-c3nc4ccccc4s3)cc2)c2ccc3cc4c(cc3c2)oc2cc(N(c3ccc(-c5nc6ccccc6s5)cc3)c3cccnc3)ccc24)c1]

The synthesis process of compound 41′ was similar to that of compound 11′ in synthesis example 8, except that the raw materials were replaced with intermediate A12 and raw material C2, with a yield of 75%.

Mass spectrum m/z: 820.207): C₅₂H₃₂N₆OS₂, C, 76.08; H, 3.93; N, 10.24; O, 1.95; S, 7.81.

¹H NMR (500 MHz, CDCl₃): δ 8.18 (2H), 8.02-8.04 (5H), 7.92 (2H), 7.85 (4H), 7.8 (1H), 7.49-7.54 (6H), 7.42 (2H), 7.36-7.37 (6H), 7.11-7.15 (3H), 6.91 (1H).

### Synthesis Example 13: Synthesis of Compound 56′

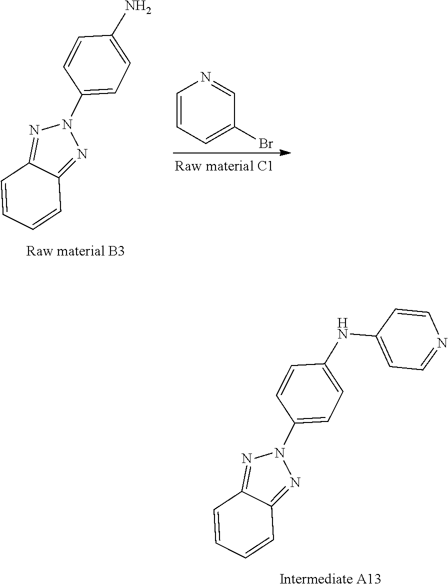 [SMILES: Nc1ccc(-n2nc3ccccc3n2)cc1 ; Brc1cccnc1 ; c1ccc2nn(-c3ccc(Nc4ccncc4)cc3)nc2c1]

400 ml of a toluene solvent was added to a reaction flask, then 0.2 mol of raw material B3, 0.2 mol of raw material C1 and 0.1 mol of sodium tert-butoxide were added, nitrogen was filled and then 0.002 mol of palladium acetate was added, nitrogen was refilled, 0.007 mol of a toluene solution of tri-tert-butyl phosphine was added; nitrogen was refilled and the reaction was refluxed for 2 hours; after the reaction was finished, the mixture was cooled to room temperature, filtered through diatomite to obtain a filtrate, the filtrate was concentrated and then methanol was added, and the mixture was left to stand for recrystallization, followed by suction filtration and rinsing with methanol to obtain a recrystallized solid, thereby intermediate A13, with a yield of 70%, was obtained.

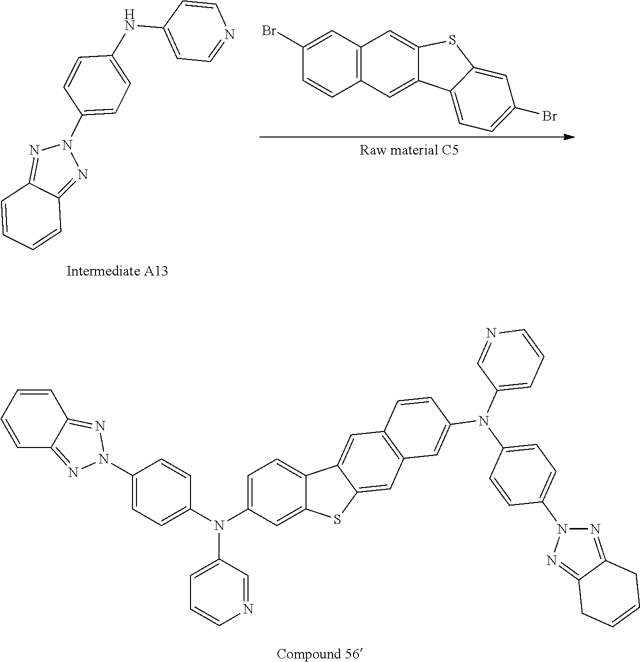

The synthesis process of compound 56′ was similar to that of compound 11′ in synthesis example 8, except that the raw materials were replaced with intermediate A13 and raw material C5, with a yield of 75%.

Mass spectrum m/z: 804.2532, element content (%): C₅₀H₃₂N₁₀S, C, 74.61; H, 4.01; N, 17.40; S, 3.98.

¹H NMR (500 MHz, CDCl₃): δ 8.03 (6H), 7.92 (3H), 7.86 (1H), 7.78 (1H), 7.64 (1H), 7.54-7.57 (9H), 7.42-7.43 (2H), 7.36 (2H), 7.11-7.18 (7).

### Refractive Index of Diarylamine Compounds

Refractive index was measured by ellipsometer; a scanning range of the instrument was 245 nm to 1000 nm; a silicon wafer was evaporated to form a thin film of a compound, a thickness of the thin film being 50 nm, and then a refractive index was measured.

Comparative compounds CP1 and CP2 were the following compounds:

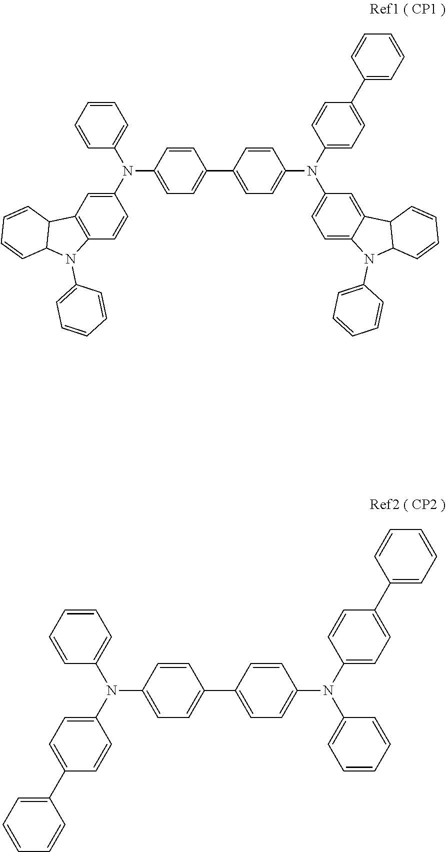 [SMILES: C1=CC2c3cc(N(c4ccccc4)c4ccc(-c5ccc(N(c6ccc(-c7ccccc7)cc6)c6ccc7c(c6)C6C=CC=CC6N7c6ccccc6)cc5)cc4)ccc3N(c3ccccc3)C2C=C1 ; c1ccc(-c2ccc(N(c3ccccc3)c3ccc(-c4ccc(N(c5ccccc5)c5ccc(-c6ccccc6)cc5)cc4)cc3)cc2)cc1]

The measurement results were shown on Table 1.

TABLE 1

| | Refractive index n at | | | | Refractive index n at | | |
|---|---|---|---|---|---|---|---|
| | different wavelengths | | | | different wavelengths | | |
| Compounds | 460 nm | 530 nm | 620 nm | Compounds | 460 nm | 530 nm | 620 nm |
| Comparative | 1.93 | 1.86 | 1.83 | Compound 1′ | 2.08 | 1.96 | 1.89 |
| Example 1 (CP1) | | | | | | | |
| Comparative | 1.99 | 1.91 | 1.87 | Compound 2′ | 2.16 | 1.98 | 1.90 |
| Example 2 (CP2) | | | | | | | |
| Compound 1 | 2.21 | 2.08 | 1.97 | Compound 3′ | 2.14 | 1.97 | 1.91 |
| Compound 2 | 2.19 | 2.05 | 1.95 | Compound 4′ | 2.11 | 1.95 | 1.88 |
| Compound 3 | 2.20 | 2.07 | 1.97 | Compound 5′ | 2.16 | 1.99 | 1.92 |
| Compound 4 | 2.22 | 2.09 | 2.00 | Compound 6′ | 2.10 | 1.97 | 1.89 |
| Compound 5 | 2.21 | 2.07 | 1.96 | Compound 7′ | 2.19 | 2.08 | 1.93 |
| Compound 6 | 2.23 | 2.10 | 1.99 | Compound 8′ | 2.24 | 2.10 | 1.96 |
| Compound 8 | 2.17 | 1.98 | 1.90 | Compound 23′ | 2.18 | 2.07 | 1.98 |
| Compound 11 | 2.24 | 2.09 | 2.00 | Compound 24′ | 2.23 | 2.12 | 2.00 |
| Compound 12 | 2.25 | 2.10 | 2.02 | Compound 31′ | 2.20 | 2.09 | 1.97 |
| Compound 13 | 2.23 | 2.07 | 1.99 | Compound 32′ | 2.24 | 2.13 | 2.01 |
| Compound 14 | 2.24 | 2.08 | 2.00 | Compound 39′ | 2.18 | 2.11 | 2.00 |
| Compound 15 | 2.20 | 1.98 | 1.90 | Compound 40′ | 2.24 | 2.13 | 2.01 |
| Compound 16 | 2.21 | 2.01 | 1.92 | Compound 46′ | 2.22 | 2.08 | 1.99 |
| Compound 17 | 2.17 | 1.96 | 1.89 | Compound 47′ | 2.19 | 2.06 | 1.97 |
| Compound 20 | 2.14 | 1.95 | 1.88 | Compound 48′ | 2.25 | 2.14 | 2.04 |
| Compound 22 | 2.18 | 2.03 | 1.95 | Compound 49′ | 2.17 | 2.06 | 1.96 |
| Compound 24 | 2.22 | 2.08 | 1.98 | Compound 50′ | 2.18 | 2.10 | 2.02 |
| Compound 25 | 2.20 | 2.00 | 1.93 | Compound 51′ | 2.20 | 2.08 | 1.98 |
| Compound 27 | 2.13 | 1.92 | 1.88 | Compound 57′ | 2.15 | 2.06 | 1.93 |
| Compound 28 | 2.14 | 1.95 | 1.90 | Compound 58′ | 2.17 | 2.08 | 1.98 |
| Compound 31 | 2.23 | 2.08 | 1.99 | Compound 59′ | 2.18 | 2.10 | 2.00 |
| Compound 35 | 2.19 | 2.05 | 1.97 | Compound 60′ | 2.20 | 2.08 | 1.99 |
| Compound 37 | 2.21 | 2.07 | 1.98 | Compound 65′ | 2.23 | 2.15 | 2.04 |
| Compound 38 | 2.23 | 2.10 | 1.99 | Compound 66′ | 2.23 | 2.16 | 2.07 |
| | | | | Compound 67′ | 2.20 | 2.12 | 2.01 |
| | | | | Compound 68′ | 2.18 | 2.09 | 1.97 |

As can be seen, compared with compounds CP1, CP2 of Comparative Examples 1, 2, the compounds of the embodiments of the present disclosure have higher refractive indices at different wavelengths. Refractive index is an important physical parameter of a light extraction material, and the magnitude of refractive index directly determines an optical coupling efficiency of a device. Therefore, the compounds of the embodiments of the present disclosure are suitable for use as a light extraction material, which may obviously improve a light output efficiency of the device, obtain a higher external quantum efficiency, reduce light loss inside the device, thus further improving the efficiency of the device.

### Light Absorption Coefficient of Diarylamine Compounds

A glass substrate was evaporated to form a thin film containing a diarylamine compound, a thickness of the thin film being 50 nm, and then light absorption coefficient was measured by a UV-Vis absorption spectrometer.

The test results were shown in Table 2.

TABLE 2

| | Light absorption coefficient k at | | | Light absorption coefficient k at | |
|---|---|---|---|---|---|
| | different wavelengths | | | different wavelengths | |
| Compounds | 400 nm | 450 nm | Compounds | 400 nm | 450 nm |
| Comparative | 0.7091 | 0 | Compound 1′ | 0.8401 | 0 |
| Example 1 | | | | | |
| (CP1) | | | | | |
| Comparative | 0.8320 | 0 | Compound 2′ | 0.8418 | 0 |
| Example 2 | | | | | |
| (CP2) | | | | | |
| Compound 1 | 0.8438 | 0 | Compound 3′ | 0.8419 | 0 |
| Compound 2 | 0.8428 | 0 | Compound 4′ | 0.8419 | 0 |
| Compound 3 | 0.8433 | 0 | Compound 5′ | 0.8419 | 0 |
| Compound 4 | 0.8441 | 0 | Compound 6′ | 0.8411 | 0 |
| Compound 5 | 0.8438 | 0 | Compound 7′ | 0.8428 | 0 |
| Compound 6 | 0.8447 | 0 | Compound 8′ | 0.8450 | 0 |
| Compound 8 | 0.8422 | 0 | Compound 23′ | 0.8446 | 0 |
| Compound 11 | 0.8450 | 0 | Compound 24′ | 0.8449 | 0 |
| Compound 12 | 0.8453 | 0 | Compound 31′ | 0.8432 | 0 |
| Compound 13 | 0.8447 | 0 | Compound 32′ | 0.8451 | 0 |
| Compound 14 | 0.8450 | 0 | Compound 39′ | 0.8445 | 0 |
| Compound 15 | 0.8433 | 0 | Compound 40′ | 0.8451 | 0 |
| Compound 16 | 0.8438 | 0 | Compound 46′ | 0.8442 | 0 |
| Compound 17 | 0.8420 | 0 | Compound 47′ | 0.8429 | 0 |
| Compound 20 | 0.8418 | 0 | Compound 48′ | 0.8453 | 0 |
| Compound 22 | 0.8425 | 0 | Compound 49′ | 0.8430 | 0 |
| Compound 24 | 0.8441 | 0 | Compound 50′ | 0.8433 | 0 |
| Compound 25 | 0.8435 | 0 | Compound 51′ | 0.8432 | 0 |
| Compound 27 | 0.8417 | 0 | Compound 57′ | 0.8419 | 0 |
| Compound 28 | 0.8418 | 0 | Compound 58′ | 0.8424 | 0 |
| Compound 31 | 0.8447 | 0 | Compound 59′ | 0.8425 | 0 |
| Compound 35 | 0.8428 | 0 | Compound 60′ | 0.8432 | 0 |
| Compound 37 | 0.8439 | 0 | Compound 65′ | 0.8446 | 0 |
| Compound 38 | 0.8448 | 0 | Compound 66′ | 0.8445 | 0 |
| | | | Compound 67′ | 0.8432 | 0 |
| | | | Compound 68″ | 0.8424 | 0 |

In order to protect an OLED device from damage by UV light in the external environment, CPL materials need to have a strong absorption capacity at about 400 nm to absorb external UV light to prevent the device from aging. At the same time, CPL materials cannot absorb light emitted by the OLED device itself, so it is required that the absorption at 450 nm or more is almost zero.

As can be seen, compared with the comparative compounds CP1 and CP2, the compounds of the embodiments of the present disclosure have significantly higher light absorption coefficients at a wavelength of 400 nm, and therefore can better absorb UV light; moreover, the compounds of the embodiments of the present disclosure have zero absorption at a wavelength of 450 nm and zero absorption at wavelengths greater than 450 nm (not shown in the table), indicating that they will not absorb light emitted by the device itself.

### Glass Transition Temperature of Diarylamine Compounds

Glass transition temperature was measured by a DSC differential scanning calorimeter; the test atmosphere was nitrogen, the heating rate was 10° C./min, and the temperature range was 50° C. to 300° C.; and the measured glass transition temperatures (Tg) were shown in Table 3.

TABLE 3

| Compounds | Tg ° C. | Compounds | Tg ° C. |
|---|---|---|---|
| Comparative | 130 | Compound 1′ | 128 |
| Example 1 (CP1) | | | |
| Comparative | 125 | Compound 2′ | 127 |
| Example 2 (CP2) | | | |
| Compound 1 | 133 | Compound 3′ | 131 |
| Compound 2 | 131 | Compound 4′ | 129 |
| Compound 3 | 134 | Compound 5′ | 130 |
| Compound 4 | 133 | Compound 6′ | 130 |
| Compound 5 | 135 | Compound 7′ | 138 |
| Compound 6 | 138 | Compound 8′ | 136 |
| Compound 8 | 137 | Compound 23′ | 134 |
| Compound 11 | 133 | Compound 24′ | 137 |
| Compound 12 | 134 | Compound 31′ | 133 |
| Compound 13 | 135 | Compound 32′ | 136 |
| Compound 14 | 136 | Compound 39′ | 138 |
| Compound 15 | 138 | Compound 40′ | 135 |
| Compound 16 | 140 | Compound 46′ | 130 |
| Compound 17 | 139 | Compound 47′ | 132 |
| Compound 20 | 132 | Compound 48′ | 138 |
| Compound 22 | 133 | Compound 49′ | 134 |
| Compound 24 | 135 | Compound 50′ | 142 |
| Compound 25 | 137 | Compound 51′ | 138 |
| Compound 27 | 139 | Compound 57′ | 131 |
| Compound 28 | 141 | Compound 58′ | 134 |
| Compound 31 | 140 | Compound 59′ | 132 |
| Compound 35 | 145 | Compound 60′ | 134 |
| Compound 37 | 134 | Compound 65′ | 139 |
| Compound 38 | 139 | Compound 66′ | 140 |
| | | Compound 67′ | 135 |
| | | Compound 68′ | 132 |

Glass transition temperature (Tg) determines thermal stability of a material in evaporation. The higher the Tg, the better the thermal stability of the material. Generally, Tg of 110° C. or above can meet the requirements of evaporation. As can be seen, the glass transition temperatures Tg of the compounds of the embodiments of the present disclosure are all high, all of them are 120° C. or above. Therefore, the compounds of the embodiments of the present disclosure are suitable for use as a light extraction material, have good stability in the evaporation process, can solve the problem of more decomposition impurities caused by unstability of materials due to heating in the evaporation process, and are beneficial to improving stability of materials in devices and the service life of the devices.

Below are testing and comparison of performance of the electroluminescent devices of some exemplary embodiments of the present disclosure.

### Performance Testing of OLED Devices

Chemical structures of some of the raw materials used were shown in Table 4.

TABLE 4

| | |
|---|---|
| p-type dopant (F4TCNQ) | 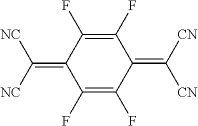 [SMILES: N#CC(C#N)=c1c(F)c(F)c(=C(C#N)C#N)c(F)c1F] |
| Material (m-MTDATA) of hole transport layer HTL and hole injection layer HIL | 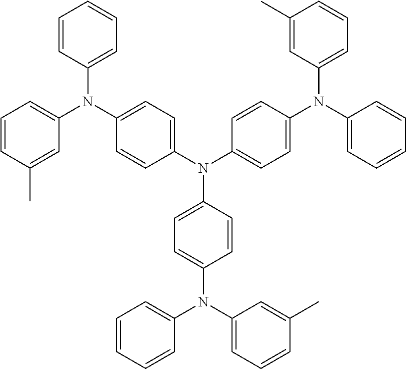 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1] |
| Material B prime (CBP) of electron block layer EBL | 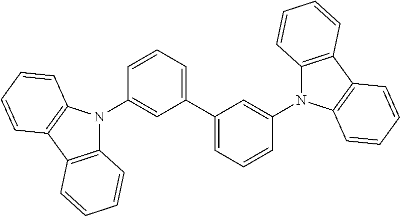 [SMILES: c1cc(-c2cccc(-n3c4ccccc4c4ccccc43)c2)cc(-n2c3ccccc3c3ccccc32)c1] |
| Material (TPBi) of hole block layer HBL | 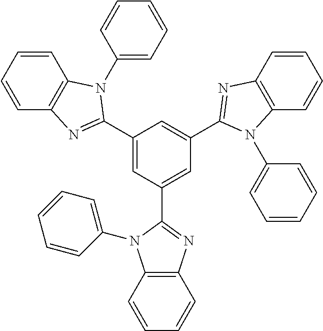 [SMILES: c1ccc(-n2c(-c3cc(-c4nc5ccccc5n4-c4ccccc4)cc(-c4nc5ccccc5n4-c4ccccc4)c3)nc3ccccc32)cc1] |
| Material (BCP) of electron transport layer ETL | 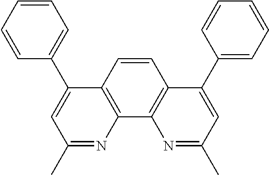 [SMILES: Cc1cc(-c2ccccc2)c2ccc3c(-c4ccccc4)cc(C)nc3c2n1] |
| Blue luminescent host material BH1 | 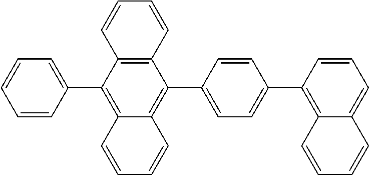 [SMILES: c1ccc(-c2c3ccccc3c(-c3ccc(-c4cccc5ccccc45)cc3)c3ccccc23)cc1] |
| Blue luminescent guest material BD1 | 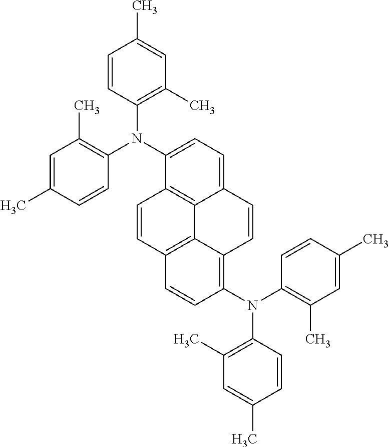 |
| Green/red luminescent host material GH/RH (CBP) | 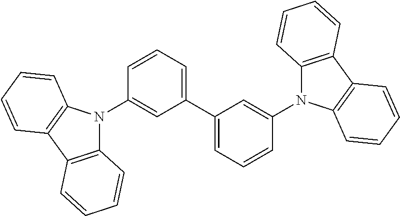 [SMILES: c1cc(-c2cccc(-n3c4ccccc4c4ccccc43)c2)cc(-n2c3ccccc3c3ccccc32)c1] |
| Green luminescent guest material GD (Ir(ppy)3) | 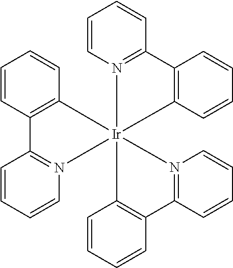 |
| Red luminescent guest material RD (Ir(piq)(acac)) | 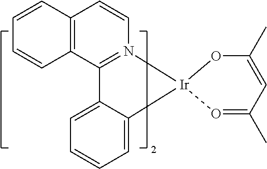 |

A preparation process of an OLED device included: cleaning and drying an ITO substrate prepared in advance; evaporating a IL material, a HTL material and an EBL material on an anode sequentially; then evaporating a material of an emitting layer, evaporating a HBL material, an ETL material and an EIL material on the emitting layer; then evaporating a cathode; and evaporating a compound of an embodiment of the present disclosure on the cathode to form a CPL layer. The device was packaged by glass UV. Thin-Film Encapsulation (TFE) may also be used, but LIF or an organic material with a refractive index n≤1.6 needs to be evaporated on CPL.

### Testing Example 1

In this testing example, a material of a CPL layer was selected from compounds 1 to 38 provided in the embodiments of the present disclosure.

### Structure and Thickness of a First Blue OLED Device

ITO/m-MTDATA: F4TCNQ 3% 10 nm/m-MTDATA 100 nm/CBP 10 nm/BH1:BD1 5% 20 nm/TPBI 5 nm/BCP:Liq 1: 1 30 nm/Yb 1 nm/Mg: Ag 13 nm/CPL 60 nm

### Structure and Thickness of a First Green OLED Device

ITO/m-MTDATA: F4TCNQ 3% 10 nm/m-MTDATA 100 nm/CBP 45 nm/GH:GD 10% 40 nm/TPBI 5 nm/BCP:Liq 1: 1 30 nm/Yb 1 nm/Mg: Ag 13 nm/CPL 60 nm

### Structure and Thickness of a First Red OLED Device

ITO/m-MTDATA:F4TCNQ 3% 10 nm/m-MTDATA 100 nm/CBP 75 nm/RH:RD 3% 45 nm/TPBI 5 nm/BCP:Liq 1: 1 30 nm/Yb 1 nm/Mg: Ag 13 nm/CPL 60 nm

The performance of the first blue OLED device was shown in Table 5.

TABLE 5

| | | | | Service life |
|---|---|---|---|---|
| | CPL material | Voltage | EQE | (LT95@1000 nit) |
| Comparative | CP1 | 100% | 100% | 100% |
| Example 1-1 | | | | |
| Comparative | CP2 | 99% | 102% | 103% |
| Example 1-2 | | | | |
| Example 1-1 | Compound 1 | 99% | 110% | 109% |
| Example 1-2 | Compound 2 | 100% | 105% | 106% |
| Example 1-3 | Compound 3 | 98% | 106% | 108% |
| Example 1-4 | Compound 4 | 99% | 112% | 109% |
| Example 1-5 | Compound 5 | 98% | 110% | 110% |
| Example 1-6 | Compound 6 | 98% | 112% | 107% |
| Example 1-7 | Compound 8 | 100% | 103% | 106% |
| Example 1-8 | Compound 11 | 99% | 113% | 109% |
| Example 1-9 | Compound 12 | 98% | 115% | 110% |
| Example 1-10 | Compound 13 | 99% | 111% | 108% |
| Example 1-11 | Compound 14 | 100% | 113% | 107% |
| Example 1-12 | Compound 15 | 99% | 105% | 106% |
| Example 1-13 | Compound 16 | 98% | 109% | 110% |
| Example 1-14 | Compound 17 | 101% | 104% | 107% |
| Example 1-15 | Compound 20 | 101% | 103% | 105% |
| Example 1-16 | Compound 22 | 99% | 105% | 108% |
| Example 1-17 | Compound 24 | 99% | 111% | 110% |
| Example 1-18 | Compound 25 | 101% | 104% | 107% |
| Example 1-19 | Compound 27 | 102% | 103% | 110% |
| Example 1-20 | Compound 28 | 101% | 104% | 108% |
| Example 1-21 | Compound 31 | 99% | 110% | 109% |
| Example 1-22 | Compound 35 | 99% | 107% | 108% |
| Example 1-23 | Compound 37 | 98% | 110% | 108% |
| Example 1-24 | Compound 38 | 99% | 112% | 106% |

As can be seen, compared with devices prepared by using CP1 and CP2 as CPL materials in the comparative examples, blue OLED devices prepared by using the compounds of the embodiments of the present disclosure as CPL materials have higher light extraction efficiency (EQE), better stability, and improved efficiency and service life.

The light extraction efficiency of red OLED devices and green OLED devices tend to be similar to that of the blue OLED devices. Therefore, using the high refractive index compounds of the embodiments of the present disclosure as the CPL materials of the OLED devices can improve the light extraction efficiency of the OLED devices. Because of the improved thermal stability of the CPL materials, the service life of the devices is relatively prolonged.

### Testing Example 2

In this testing example, a material of a CPL layer was selected from compounds 1′ to 69′ provided in the embodiments of the present disclosure.

### Structure and Thickness of a Second Blue OLED Device

ITO/m-MTDATA:F4TCNQ 3% 10 nm/m-MTDATA 1 nm/CBP 5 nmBH1:BD1 5% 20 nm/TPBI 5 nm/BCP:Liq 1: 1 30 nm/Yb 1 nm/Mg: Ag 13 nm/CPL 65 nm

### Structure and Thickness of a Second Green OLED Device

ITO/m-MTDATA:F4TCNQ 3% 10 nm/m-MTDATA 110 nm/CBP 45 nm/GH:GD 10% 40 nm/TPBI 5 nm/BCP:Liq 1: 1 30 nm/Yb 1 nm/Mg: Ag 3 nm/CPL 65 nm

### Structure and Thickness of a Second Red OLED Device

ITO/m-MTDATA:F4TCNQ 3% 10 nm/m-MTDATA 100 nm/CBP 75 nm/RH:RD 3% 45 nm/TPBI 5 nm/BCP:Liq 1: 1 30 nm/Yb 1 nm/Mg: Ag 13 nm/CPL 65 nm

The performance of the second blue OLED device was shown in Table 6.

TABLE 6

| | | | | Service life |
|---|---|---|---|---|
| | CPL material | Voltage | EQE | (LT95@1000 nit) |
| Comparative | CP1 | 100% | 100% | 100% |
| Example 2-1 | | | | |
| Comparative | CP2 | 99% | 102% | 103% |
| Example 2-2 | | | | |
| Example 2-1 | Compound 11′ | 99% | 106% | 108% |
| Example 2-2 | Compound 12′ | 100% | 110% | 108% |
| Example 2-3 | Compound 13′ | 99% | 113% | 109% |
| Example 2-4 | Compound 44′ | 99% | 118% | 107% |
| Example 2-5 | Compound 56′ | 99% | 111% | 104% |

The performance of the second green OLED device was shown in Table 7.

TABLE 7

| | | | | Service life |
|---|---|---|---|---|
| | CPL material | Voltage | EQE | (LT95@1000 nit) |
| Comparative | CP1 | 100% | 100% | 100% |
| Example 3-1 | | | | |
| Example 3-1 | Compound 11′ | 100% | 108% | 109% |
| Example 3-2 | Compound 12′ | 99% | 112% | 108% |
| Example 3-3 | Compound 32′ | 100% | 112% | 108% |

The performance of the second red OLED device was shown in Table 8.

TABLE 8

| | | | | Service life |
|---|---|---|---|---|
| | CPL material | Voltage | EQE | (LT95@1000 nit) |
| Comparative | CP2 | 100% | 100% | 100% |
| Example 4-2 | | | | |
| Example 4-1 | Compound 13′ | 100% | 104% | 106% |
| Example 4-2 | Compound 44′ | 99% | 107% | 105% |
| Example 4-3 | Compound 56′ | 99% | 110% | 107% |

As can be seen, compared with the devices prepared by using CP1 and CP2 as CPL materials in the comparative examples, the devices prepared by using the diarylamine compounds of the embodiments of the present disclosure as CPL materials have higher light extraction efficiency (EQE), certain improvement in stability, and improvement in efficiency and service life.

Although the implementation modes disclosed in the present disclosure are as above, the described contents are only implementation modes used for convenience of understanding the present disclosure and are not intended to limit the present disclosure. Any skilled person in the art to which the present disclosure pertains may make any modifications and alterations in forms and details of implementation without departing from the spirit and scope of the present disclosure. However, the scope of patent protection of the present application should still be subject to the scope defined by the appended claims.

1. A diarylamine compound, having a general structural formula: 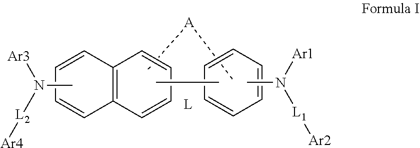
wherein A is not present or present;
i) when A is not present, a naphthalene ring is linked with a benzene ring by a bond L;
at least two of Ar₁ to Ar₄ comprise groups represented by General Formula II and/or General Formula III: 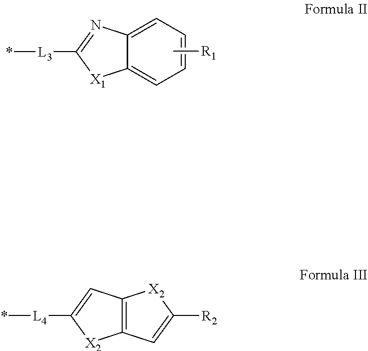
the remaining of Ar₁ to Ar₄ that do not comprise groups represented by General Formula II or General Formula III comprise any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
X₁ comprises CR₃R₄, O, NR₅ or S; X₂ comprises O or S;
R₁ to R₅ each independently comprise any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 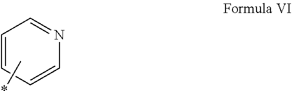
Y₁ and Z₁ each independently comprise any one of CR₁₂R₁₃, O, NR₁₄ and S, Y₂ and Z₂ each independently comprise C or N, and Y₃ and Z₃ each comprise N;
the remaining of Ar₁ to Ar₄ that do not comprise groups represented by General Formulas IV, V and VI comprise any one of substituted or unsubstituted C6 to C60 aryl, and substituted or unsubstituted C5 to C60 heteroaryl; where the substituted C6 to C60 aryl and substituted C5 to C60 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C60 aryl and C5 to C60 heteroaryl;
R₆ to R₁₄ each independently comprise any one of hydrogen, heavy hydrogen, halogen, nitro, nitrile, substituted or unsubstituted C1 to C30 alkyl, substituted or unsubstituted C2 to C30 alkenyl, substituted or unsubstituted C1 to C30 alkoxy, substituted or unsubstituted C1 to C30 thioether, substituted or unsubstituted C6 to C50 aryl, and substituted or unsubstituted C2 to C50 heteroaryl; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ and L₂ each independently comprise any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene.

2. The diarylamine compound according to claim 1, wherein the group represented by General Formula II comprises any one of the following groups:

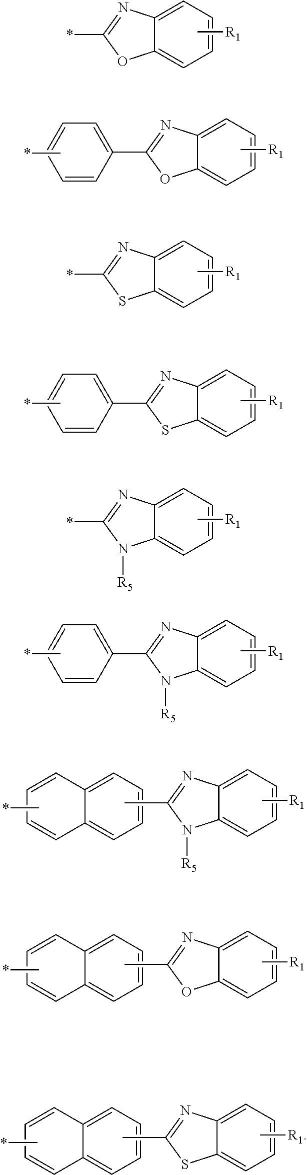

3. The diarylamine compound according to claim 2, wherein the group represented by General Formula II comprises any one of the following groups:

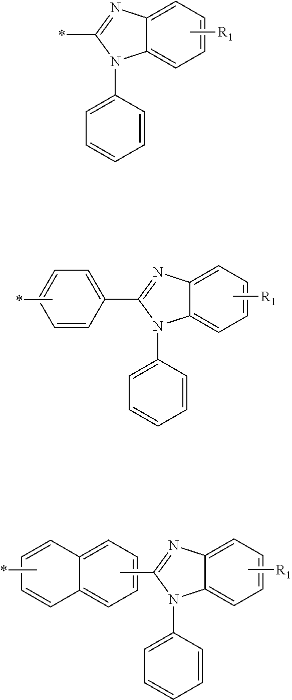

4. The diarylamine compound according to claim 1, wherein the group represented by General Formula IV comprises: 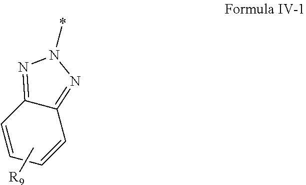
at least one of Ar₁ to Ar₄ comprises a group represented by General Formula IV-1.

5. The diarylamine compound according to claim 1, wherein the group represented by General Formula V comprises: 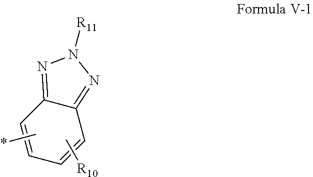
at least one of Ar₁ to Ar₄ comprises a group represented by General Formula V-1.

6. The diarylamine compound according to claim 1, wherein the group represented by General Formula IV comprises: 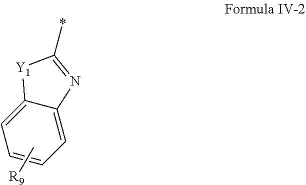
the group represented by General Formula V comprises: 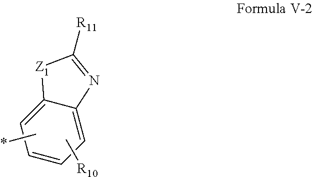
at least one of Ar₁ to Ar₄ comprises a group represented by General Formula IV-2 or V-2.

7. The diarylamine compound according to claim 6, wherein
the group represented by General Formula IV-2 comprises: 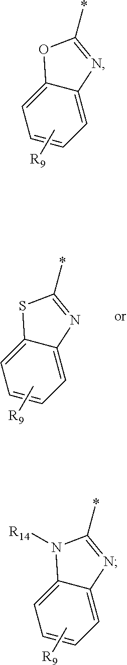
or
the group represented by General Formula V-2 comprises:

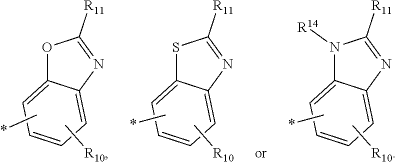

8. The diarylamine compound according to claim 1, wherein the diarylamine compound comprises any one of the following compounds: 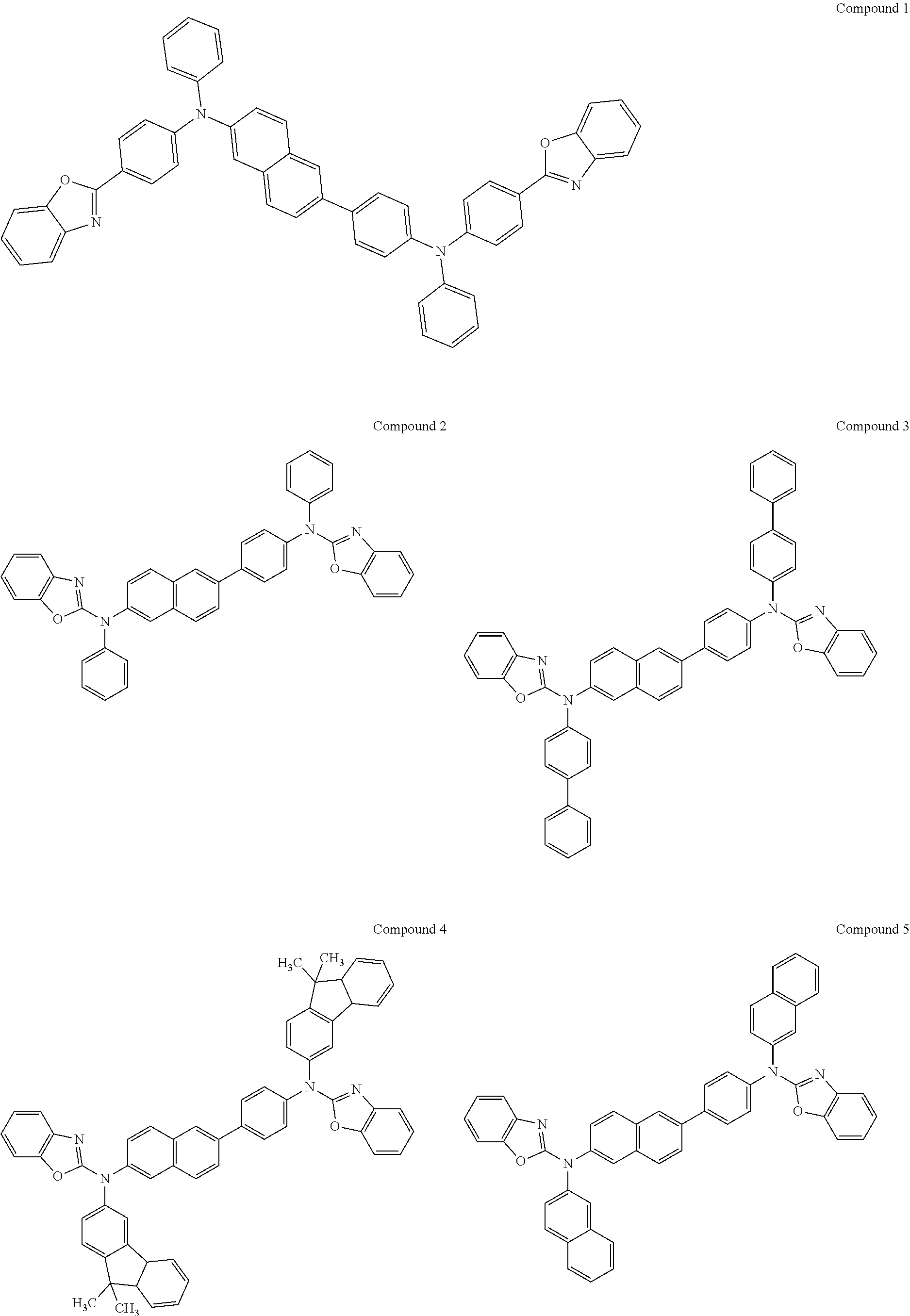 [SMILES: c1ccc(N(c2ccc(-c3ccc4cc(N(c5ccccc5)c5ccc(-c6nc7ccccc7o6)cc5)ccc4c3)cc2)c2ccc(-c3nc4ccccc4o3)cc2)cc1 ; c1ccc(N(c2ccc(-c3ccc4cc(N(c5ccccc5)c5nc6ccccc6o5)ccc4c3)cc2)c2nc3ccccc3o2)cc1 ; c1ccc(-c2ccc(N(c3ccc(-c4ccc5cc(N(c6ccc(-c7ccccc7)cc6)c6nc7ccccc7o6)ccc5c4)cc3)c3nc4ccccc4o3)cc2)cc1 ; CC1(C)c2ccc(N(c3ccc(-c4ccc5cc(N(c6ccc7c(c6)C6C=CC=CC6C7(C)C)c6nc7ccccc7o6)ccc5c4)cc3)c3nc4ccccc4o3)cc2C2C=CC=CC21 ; c1ccc2cc(N(c3ccc(-c4ccc5cc(N(c6ccc7ccccc7c6)c6nc7ccccc7o6)ccc5c4)cc3)c3nc4ccccc4o3)ccc2c1] 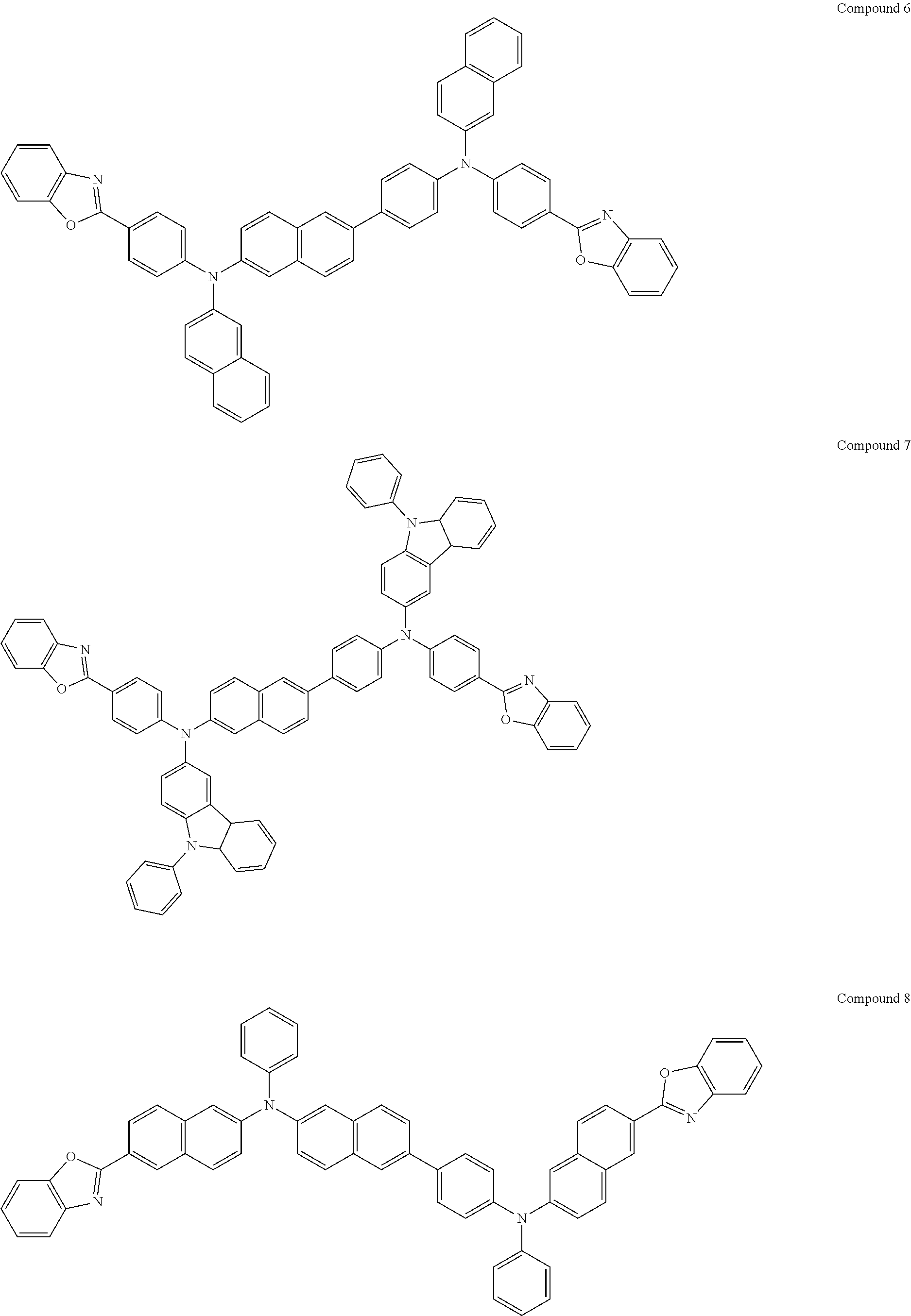 [SMILES: c1ccc2cc(N(c3ccc(-c4ccc5cc(N(c6ccc(-c7nc8ccccc8o7)cc6)c6ccc7ccccc7c6)ccc5c4)cc3)c3ccc(-c4nc5ccccc5o4)cc3)ccc2c1 ; C1=CC2c3cc(N(c4ccc(-c5ccc6cc(N(c7ccc(-c8nc9ccccc9o8)cc7)c7ccc8c(c7)C7C=CC=CC7N8c7ccccc7)ccc6c5)cc4)c4ccc(-c5nc6ccccc6o5)cc4)ccc3N(c3ccccc3)C2C=C1 ; c1ccc(N(c2ccc(-c3ccc4cc(N(c5ccccc5)c5ccc6cc(-c7nc8ccccc8o7)ccc6c5)ccc4c3)cc2)c2ccc3cc(-c4nc5ccccc5o4)ccc3c2)cc1] 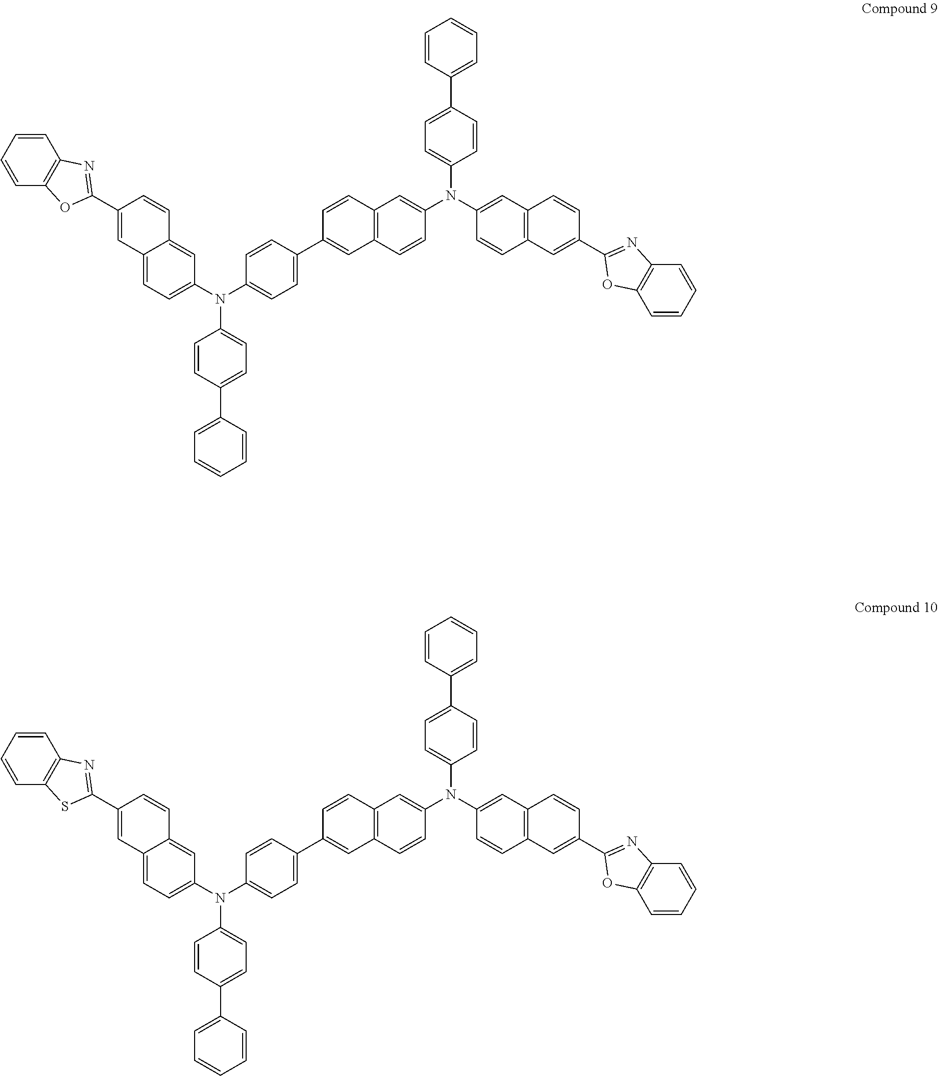 [SMILES: c1ccc(-c2ccc(N(c3ccc(-c4ccc5cc(N(c6ccc(-c7ccccc7)cc6)c6ccc7cc(-c8nc9ccccc9o8)ccc7c6)ccc5c4)cc3)c3ccc4cc(-c5nc6ccccc6o5)ccc4c3)cc2)cc1 ; c1ccc(-c2ccc(N(c3ccc4cc(-c5ccc(N(c6ccc(-c7ccccc7)cc6)c6ccc7cc(-c8nc9ccccc9s8)ccc7c6)cc5)ccc4c3)c3ccc4cc(-c5nc6ccccc6o5)ccc4c3)cc2)cc1]
to C50 heteroaryl, and when R₁ and R₂ each independently comprise substituted or unsubstituted C6 to C50 aryl and substituted or unsubstituted C2 to C50 heteroaryl, R₁ or R₂ and a benzene ring in General Formula II or III are respectively linked by a single bond or fused by sharing two atoms; where the substituted C1 to C30 alkyl, substituted C2 to C30 alkenyl, substituted C1 to C30 alkoxy, substituted C1 to C30 thioether, substituted C6 to C50 aryl, and substituted C2 to C50 heteroaryl mean being substituted by one or more of the following groups: heavy hydrogen, halogen, nitro, nitrile, C1 to C30 alkyl, C2 to C30 alkenyl, C1 to C30 alkoxy, C1 to C30 thioether, C6 to C50 aryl, and C2 to C50 heteroaryl;
L₁ to L₄ each independently comprise any one of a single bond, substituted or unsubstituted C6 to C50 arylene, substituted or unsubstituted C2 to C50 heteroarylene, where the substituted C6 to C50 arylene and substituted C2 to C50 heteroarylene mean being substituted by one or more of the following groups: heavy hydrogen, halogen, nitro, nitrile, C6 to C50 aryl, C2 to C50 heteroarylene;
ii) when A is present, A forms a five-membered ring with the bond L, and A comprises any one of O, S, NR₆ and CR₇R₈;
at least one of Ar₁ to Ar₄ comprises a group represented by General Formula IV or V: 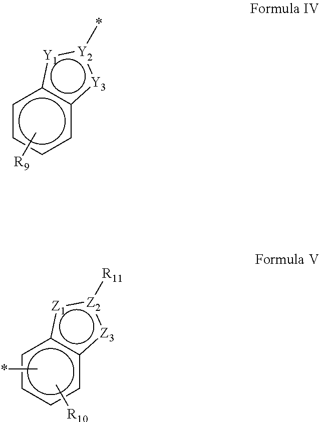
and at least one of Ar₁ to Ar₄ comprises a group represented by General Formula VI:

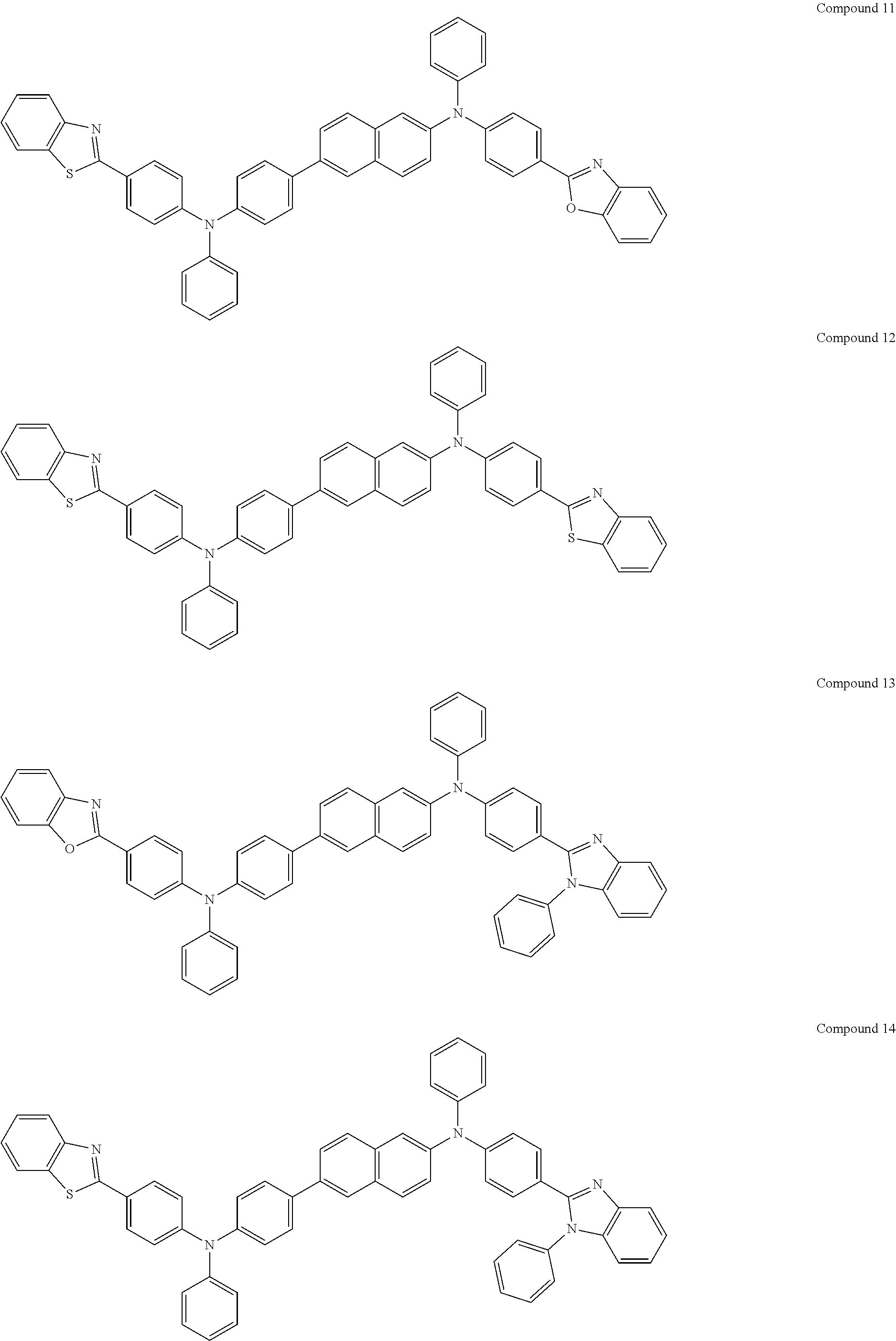

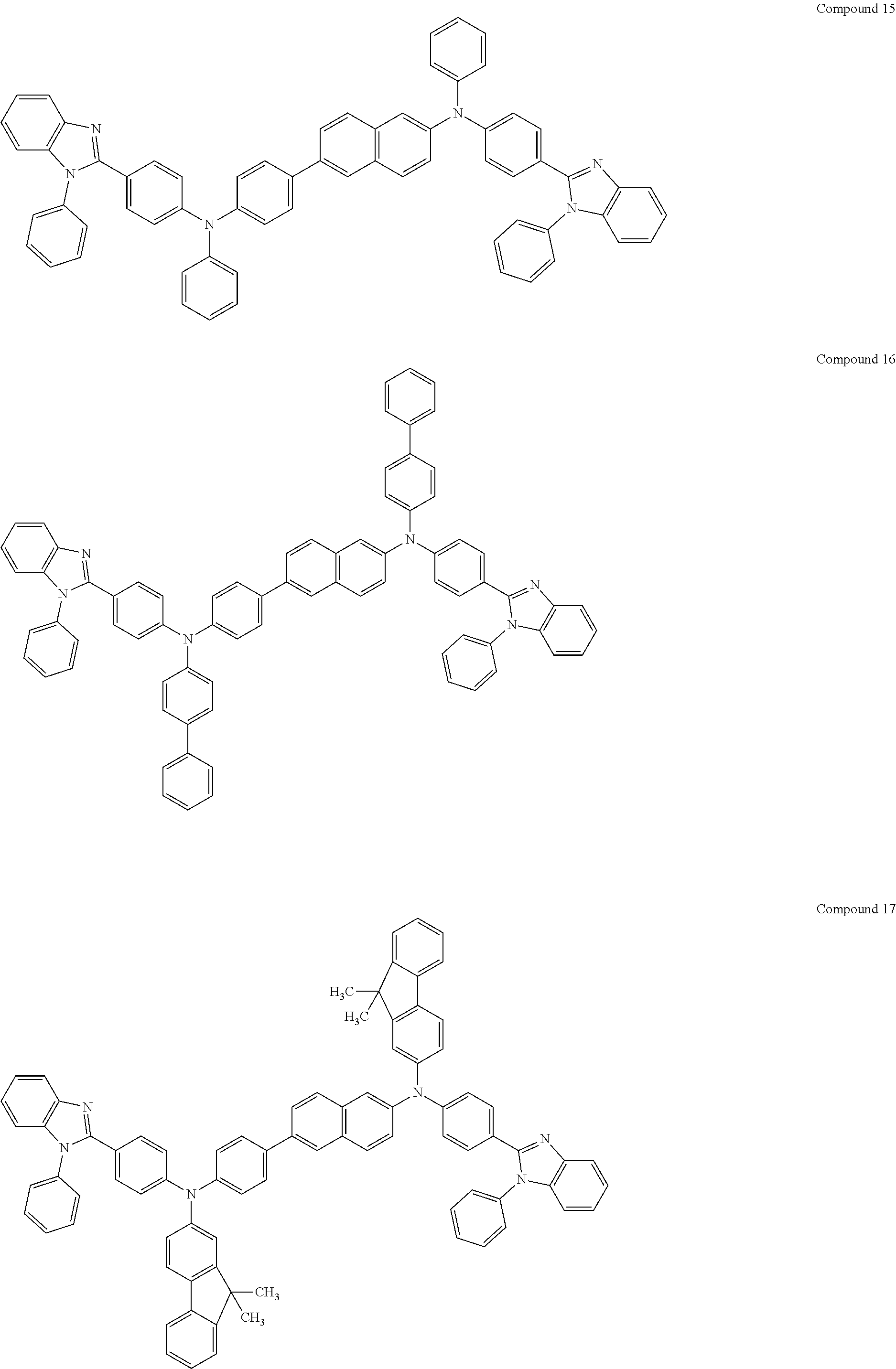

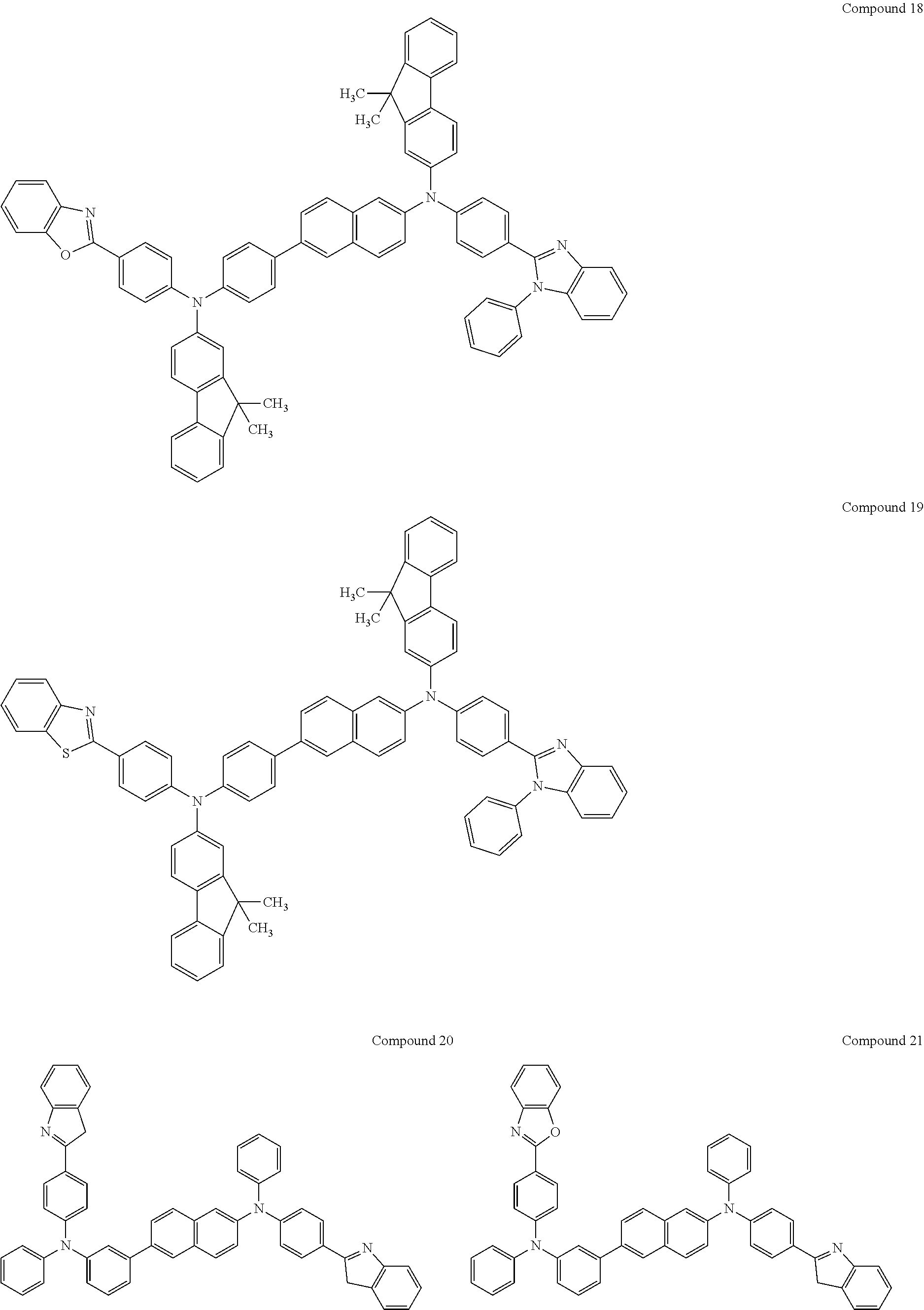

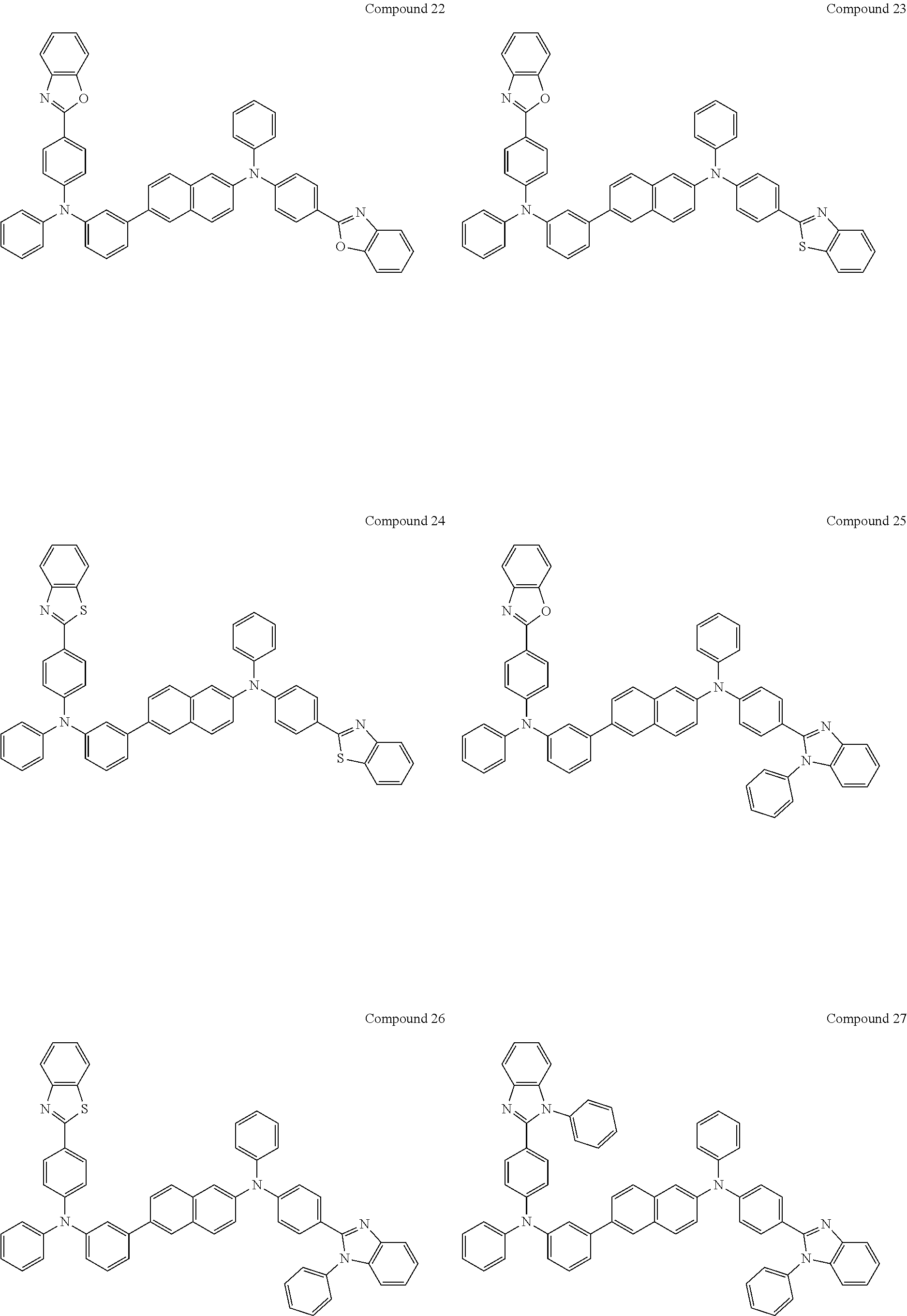

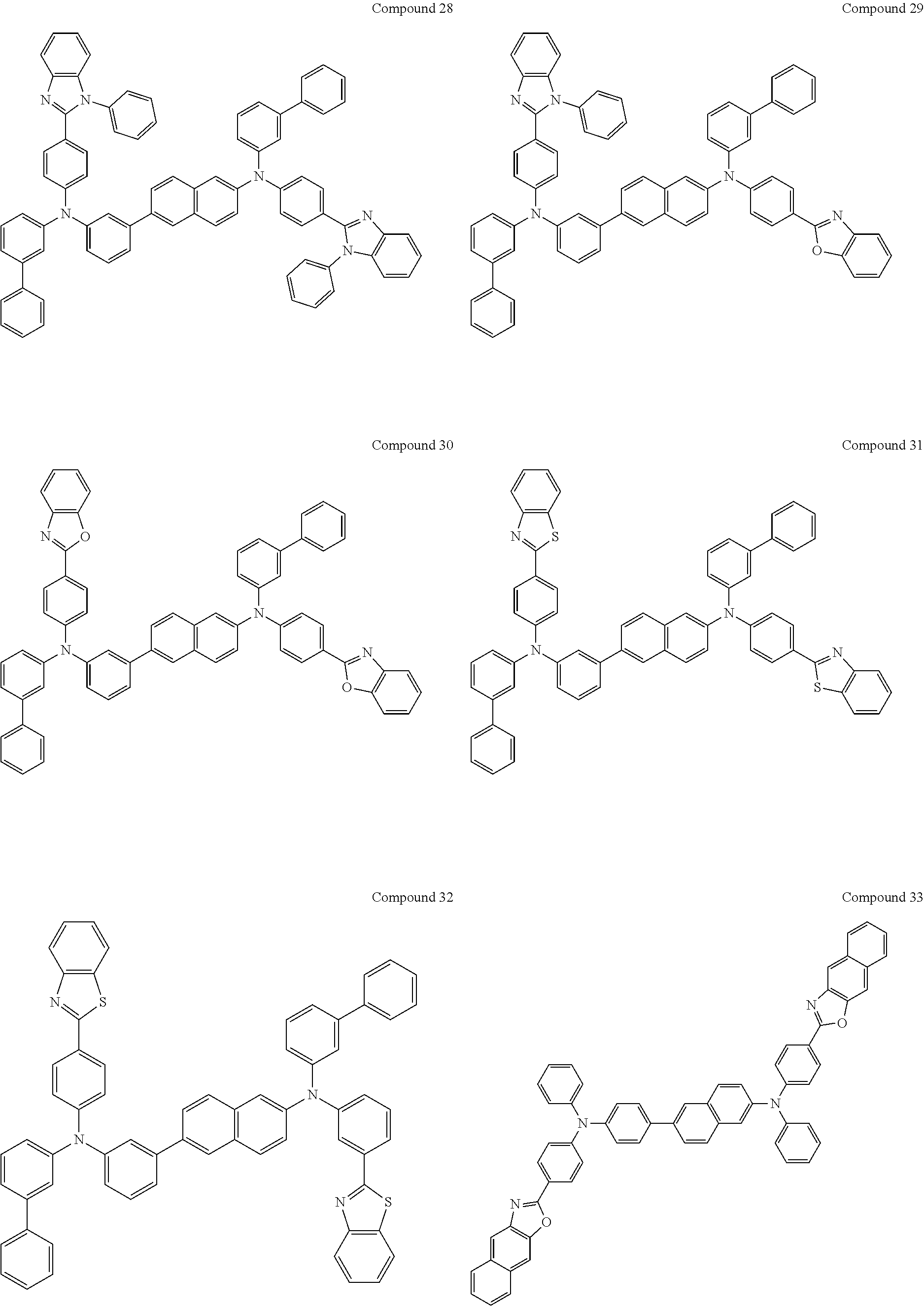

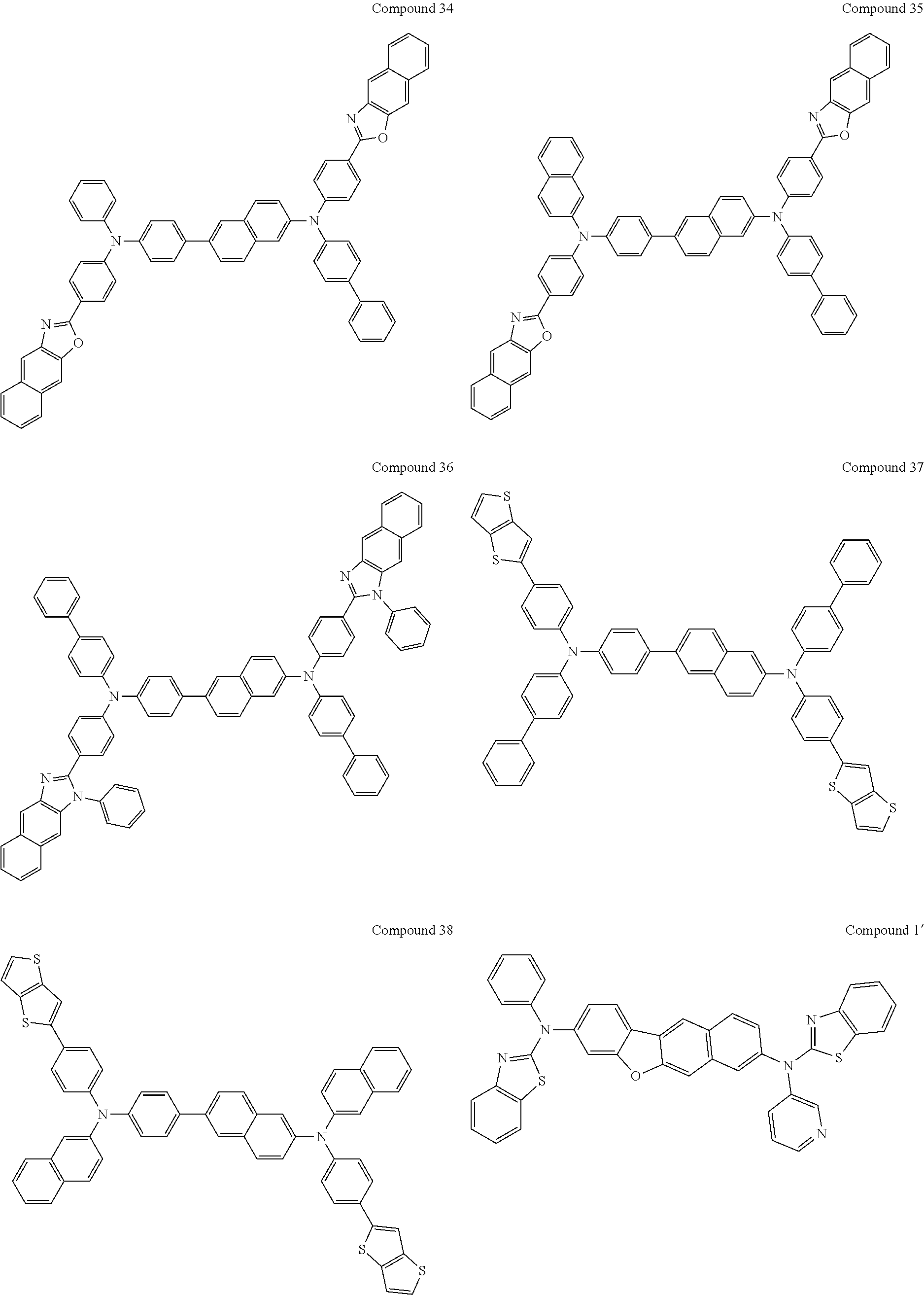

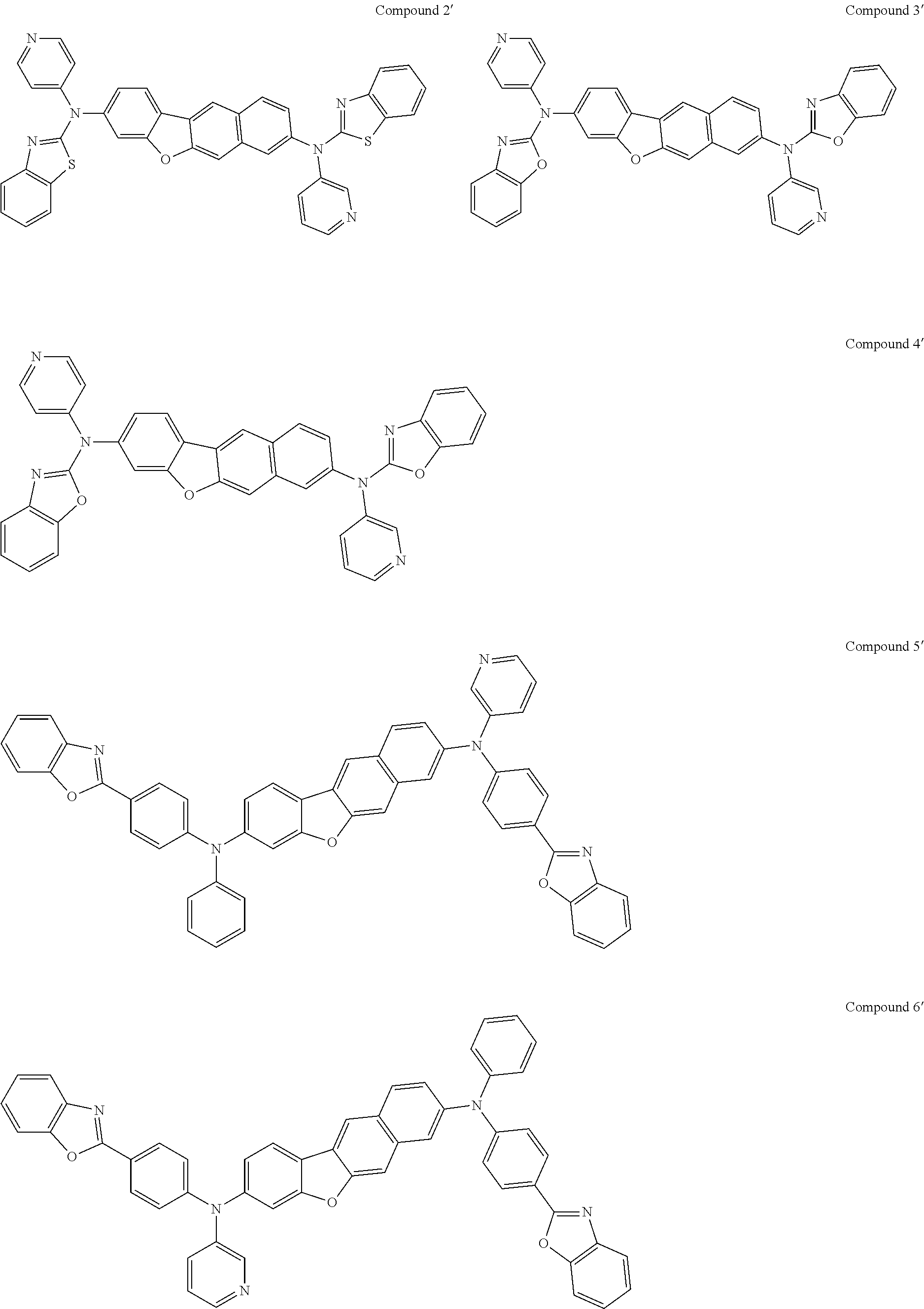

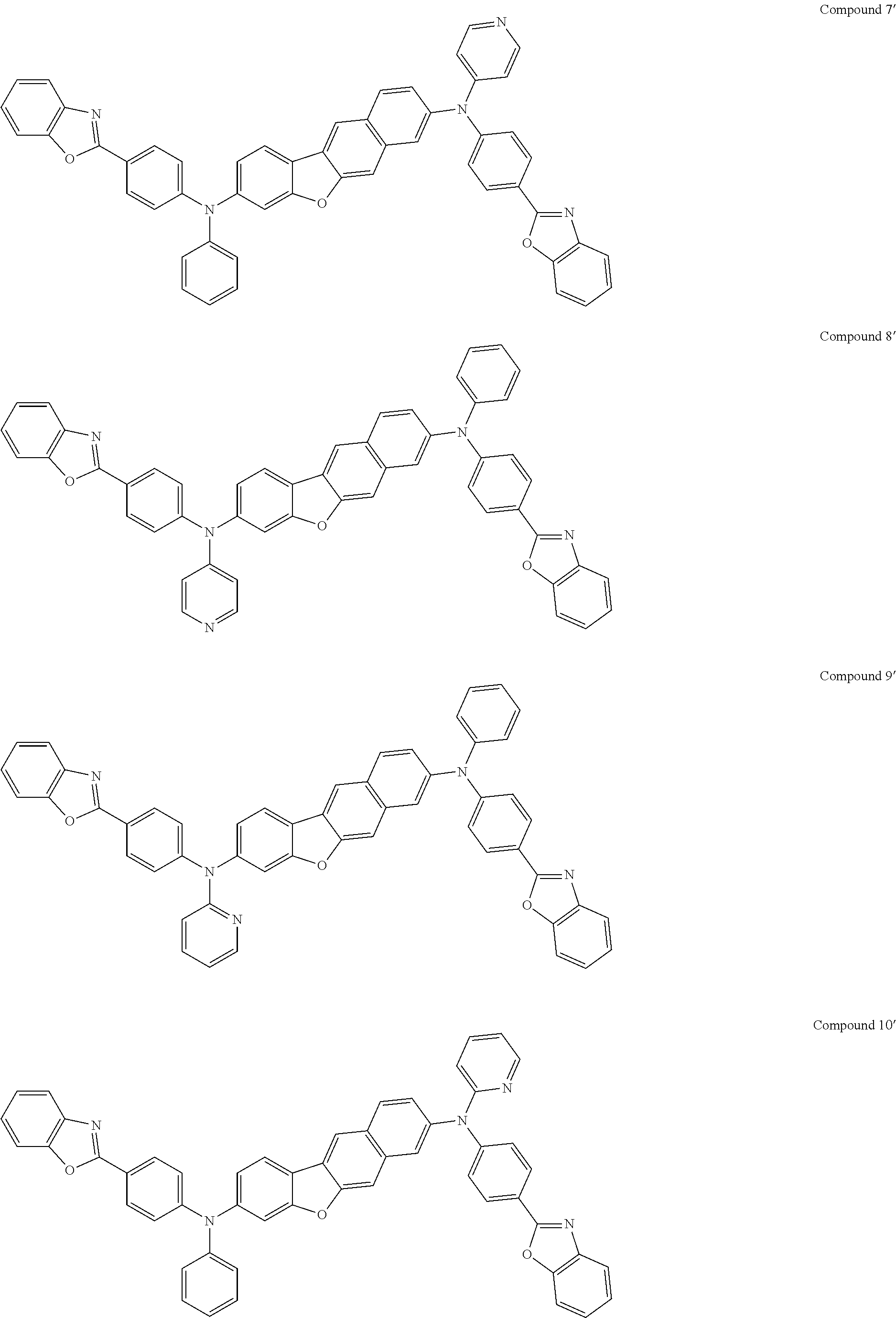

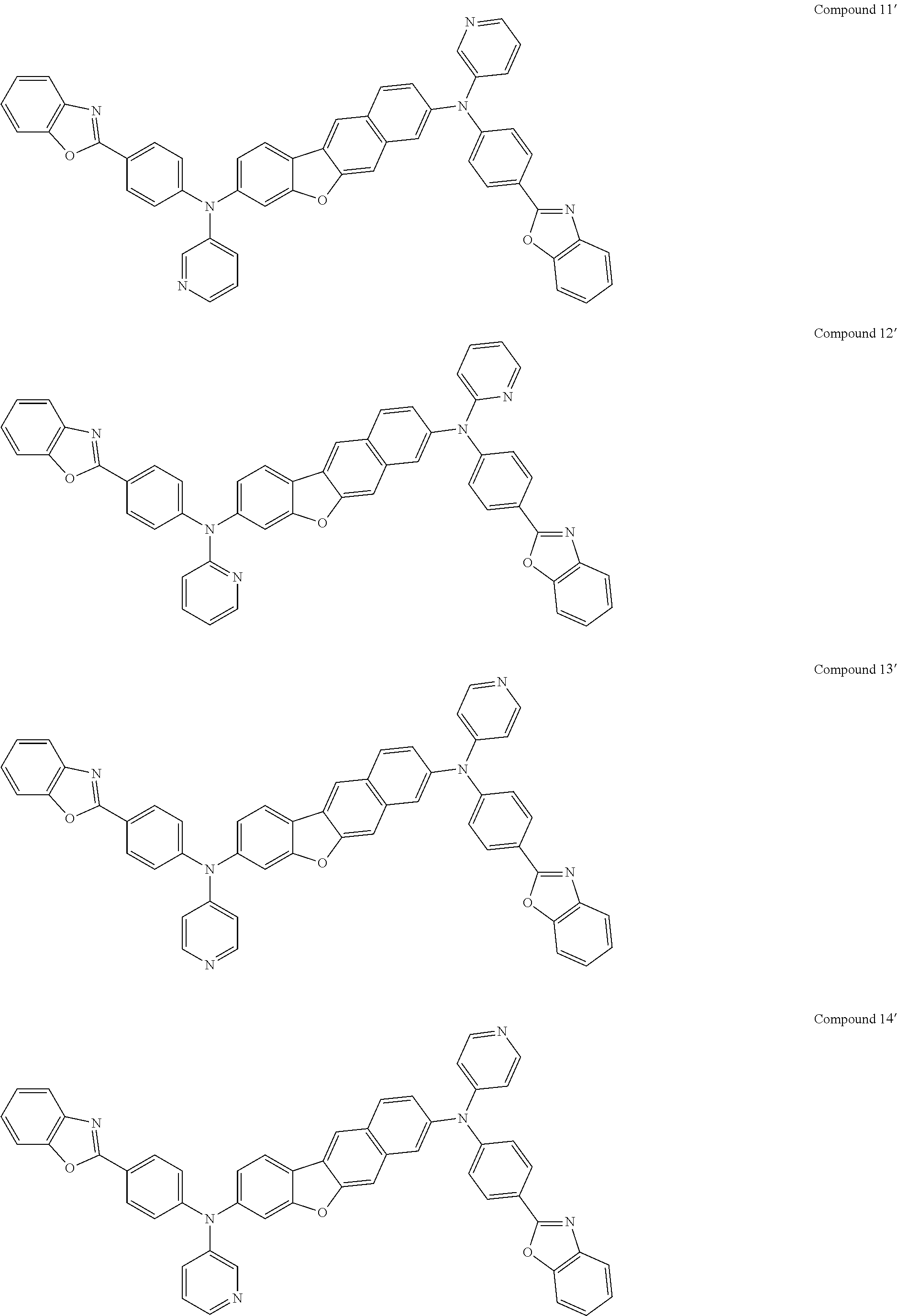

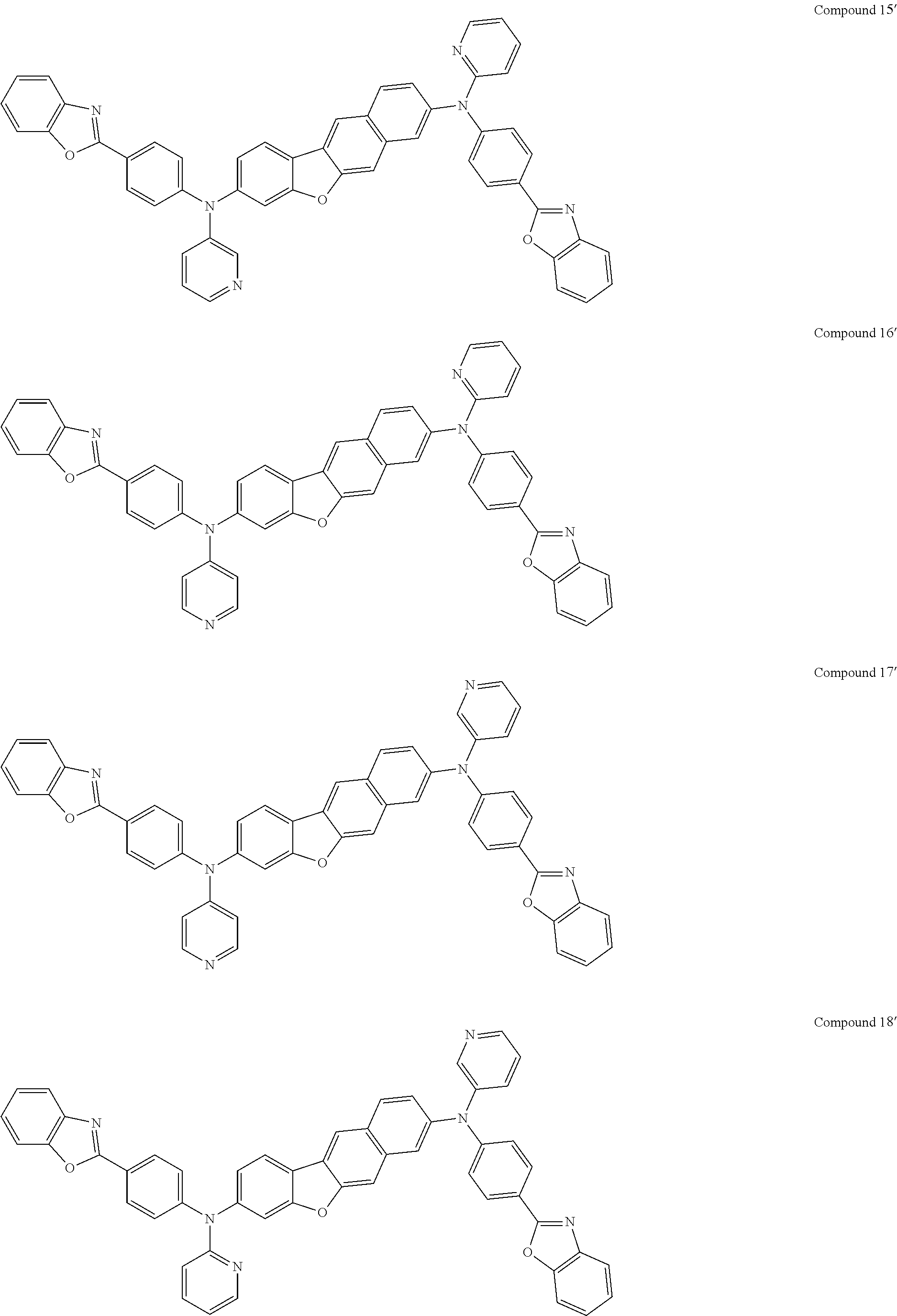

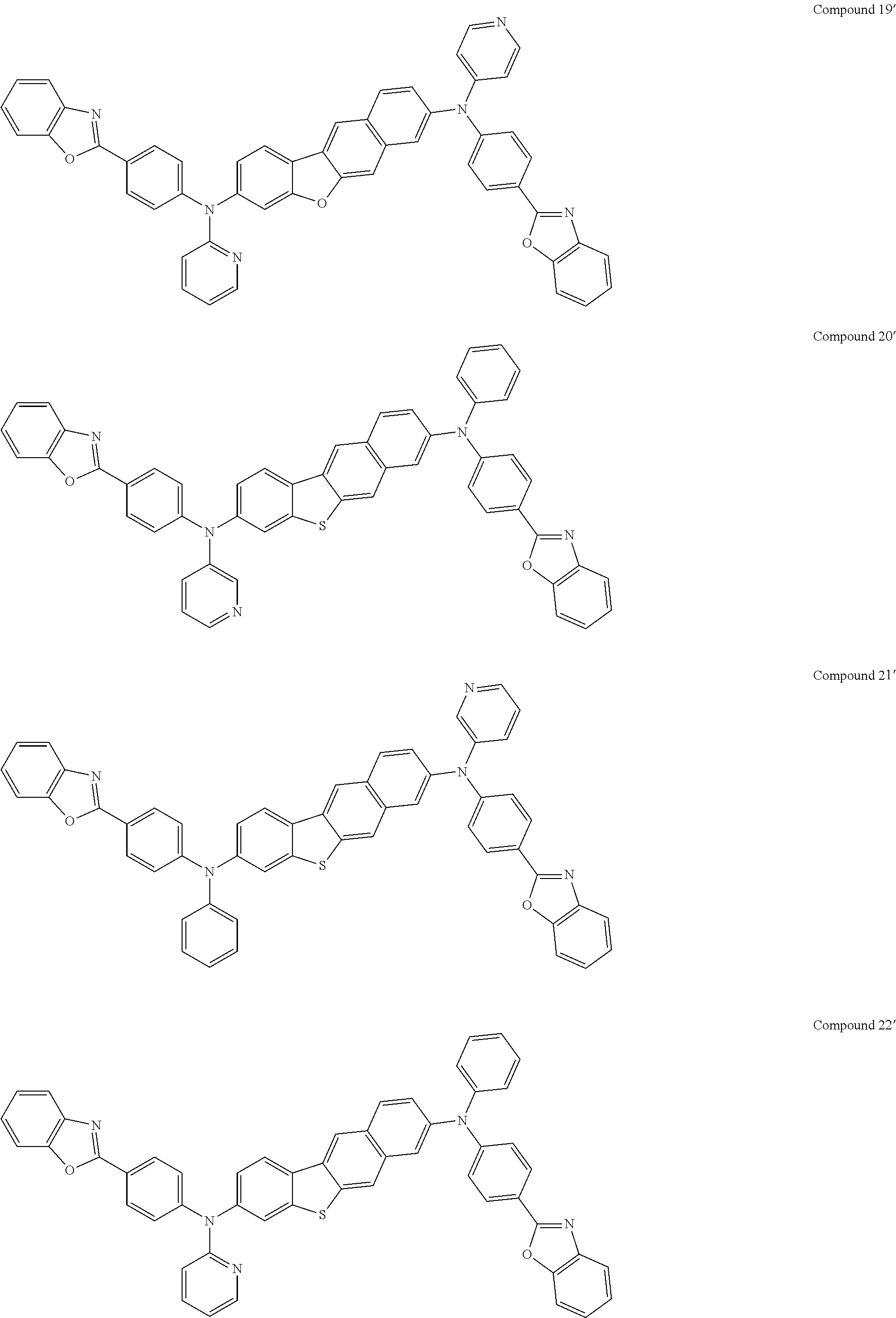

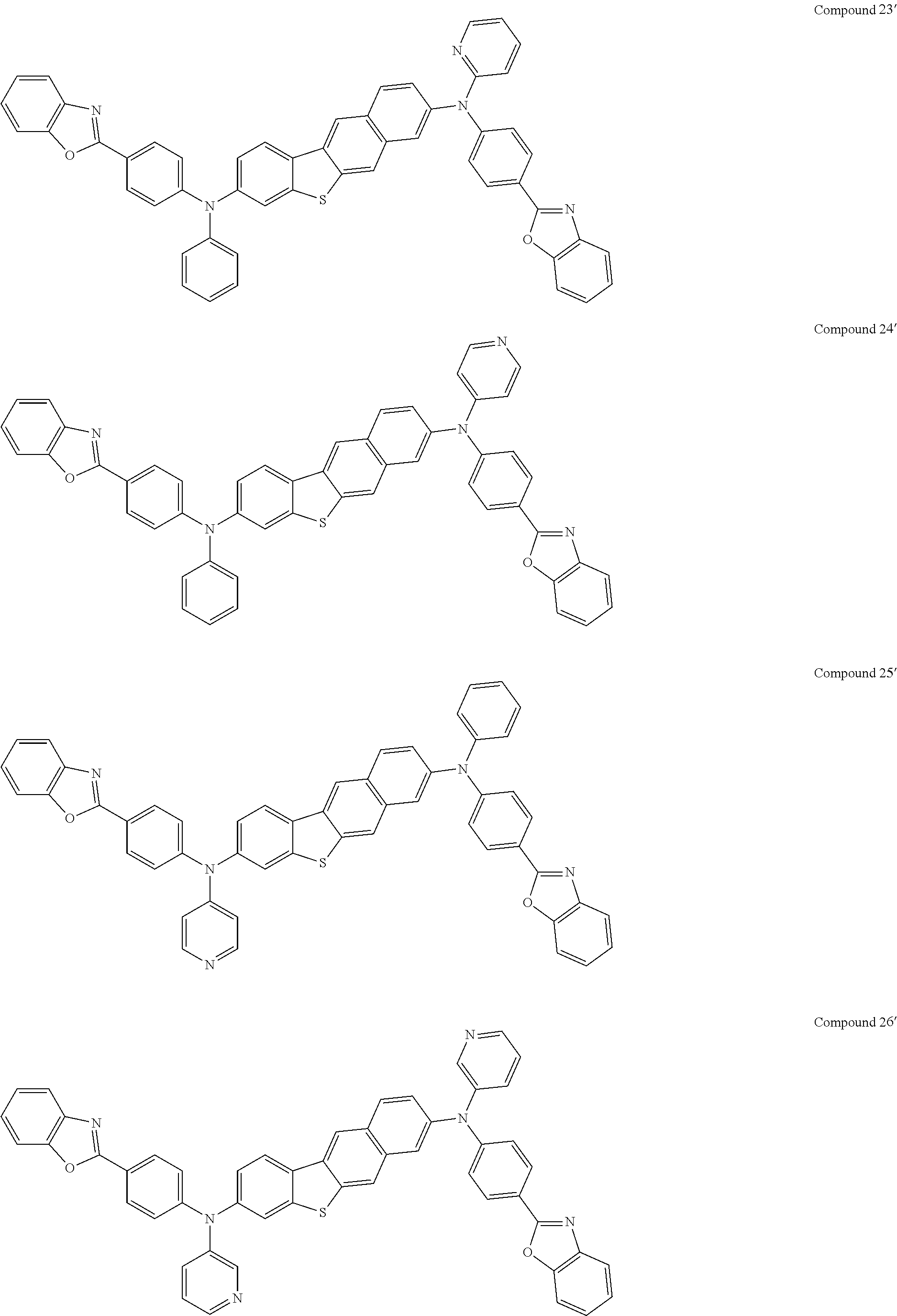

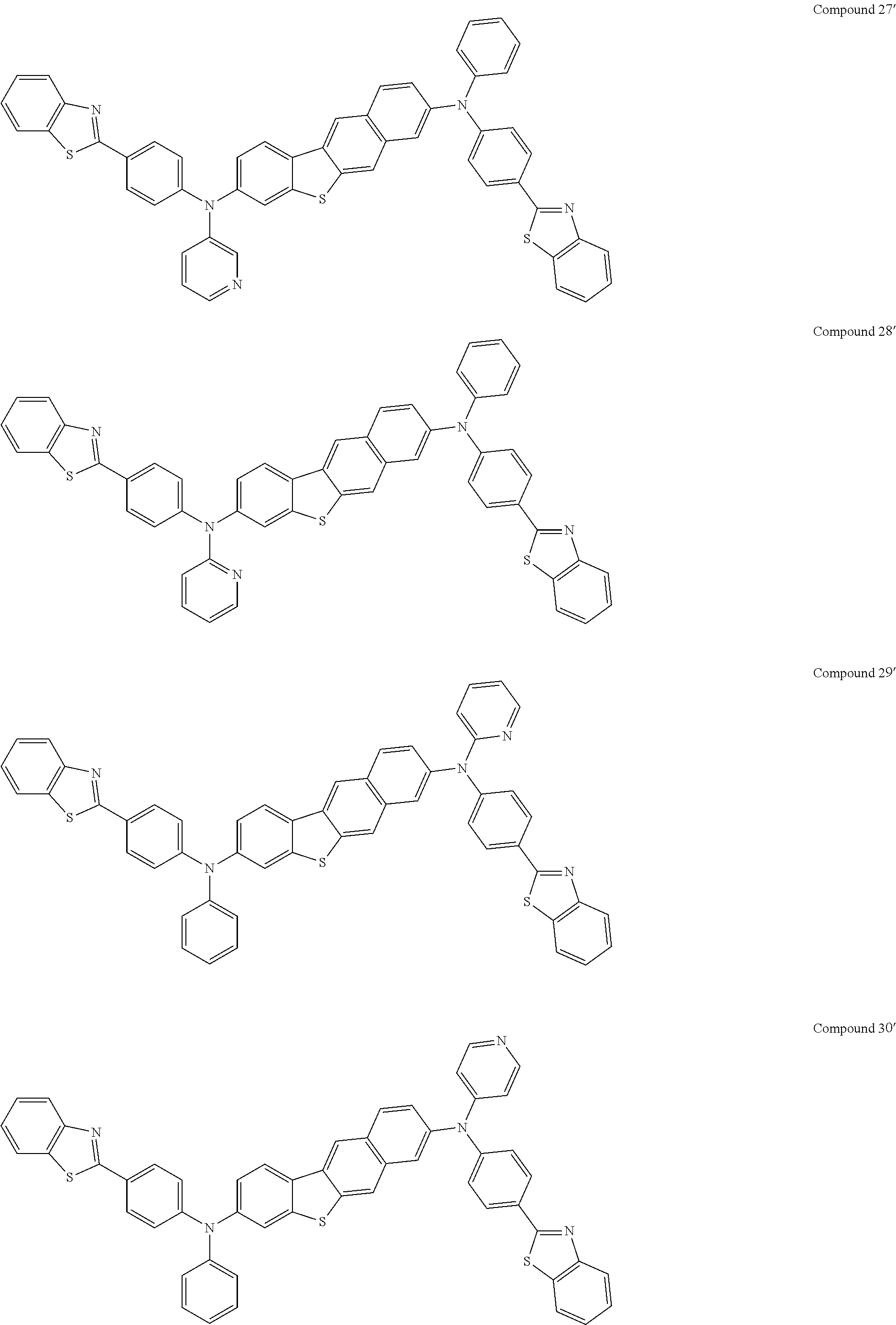

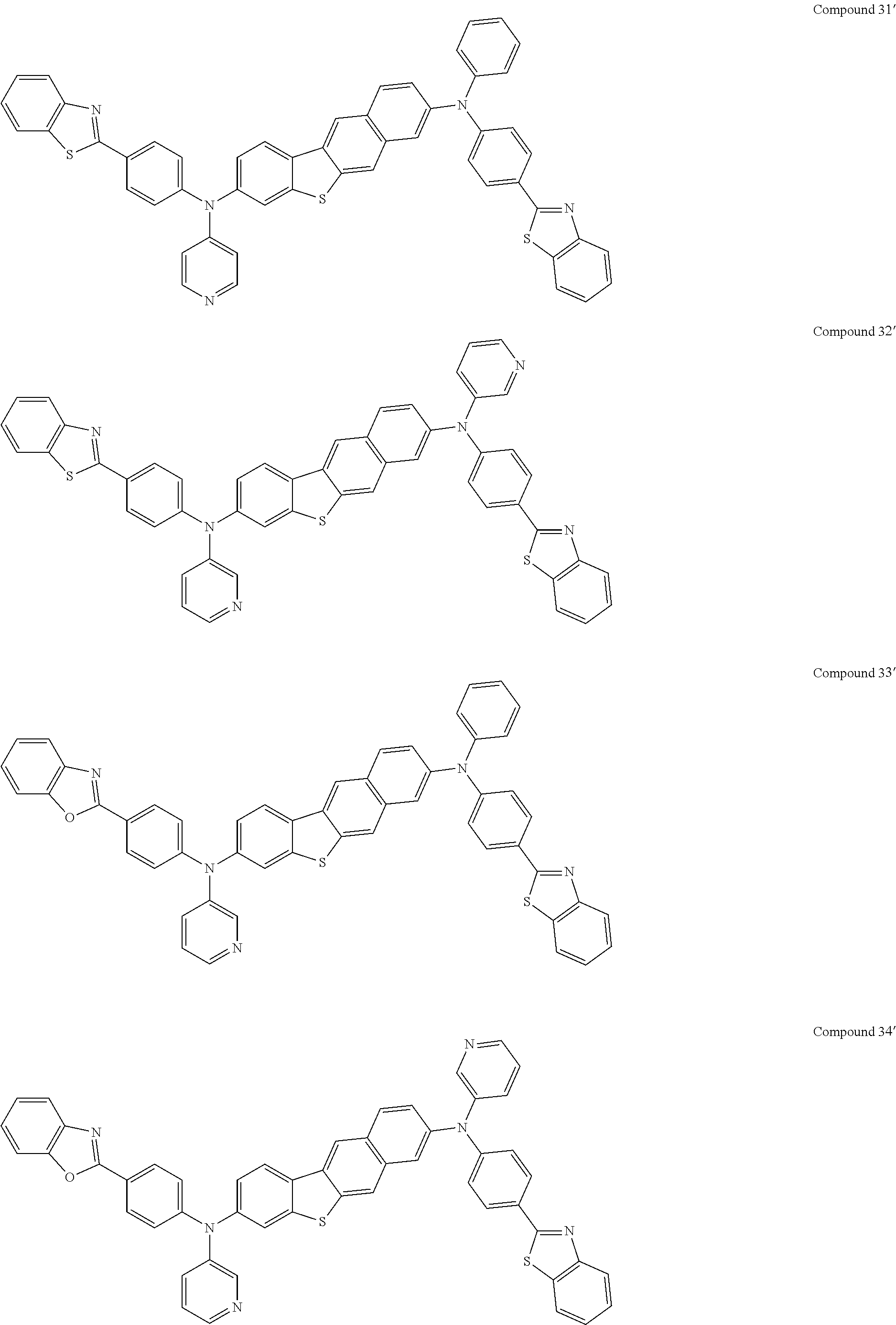

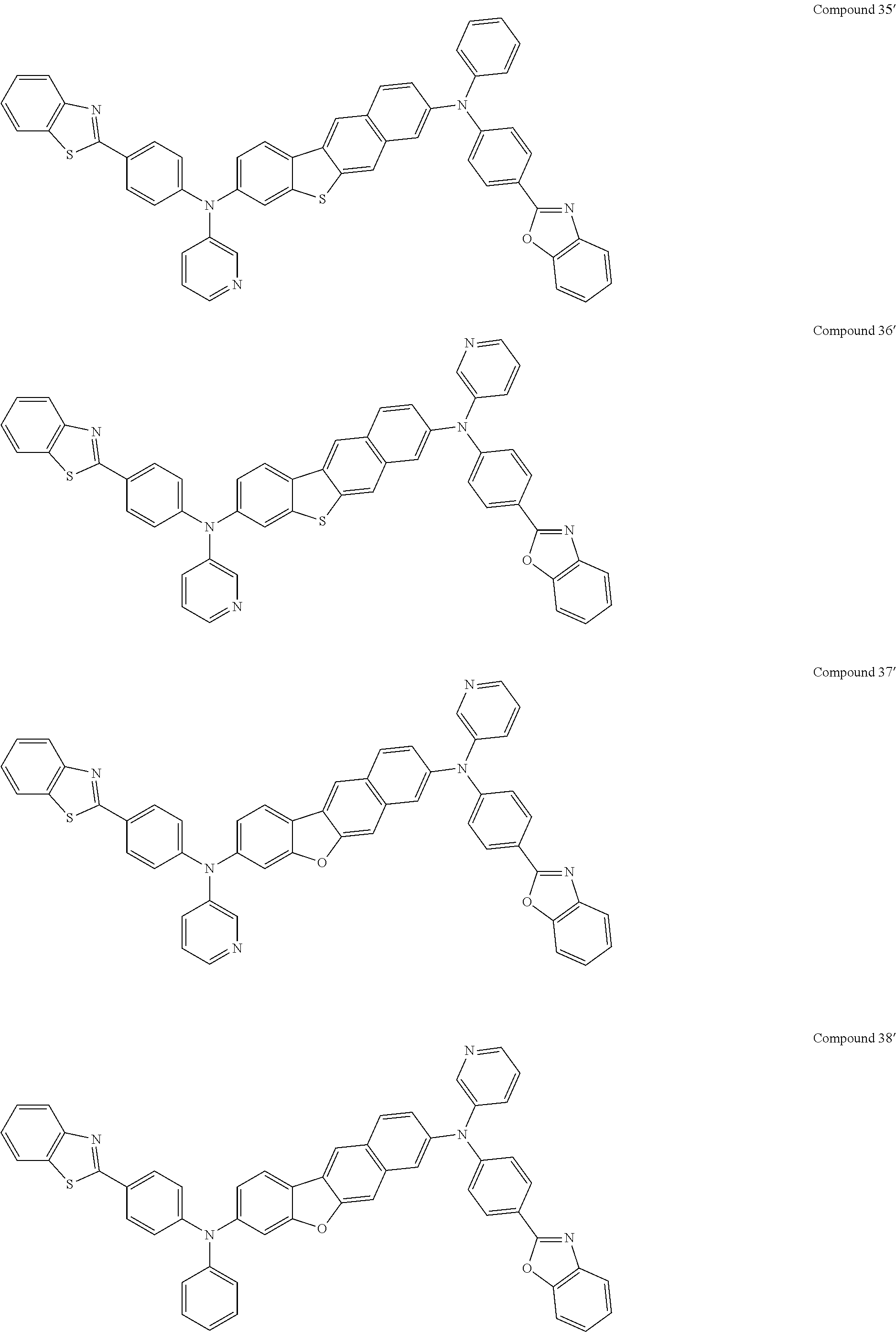

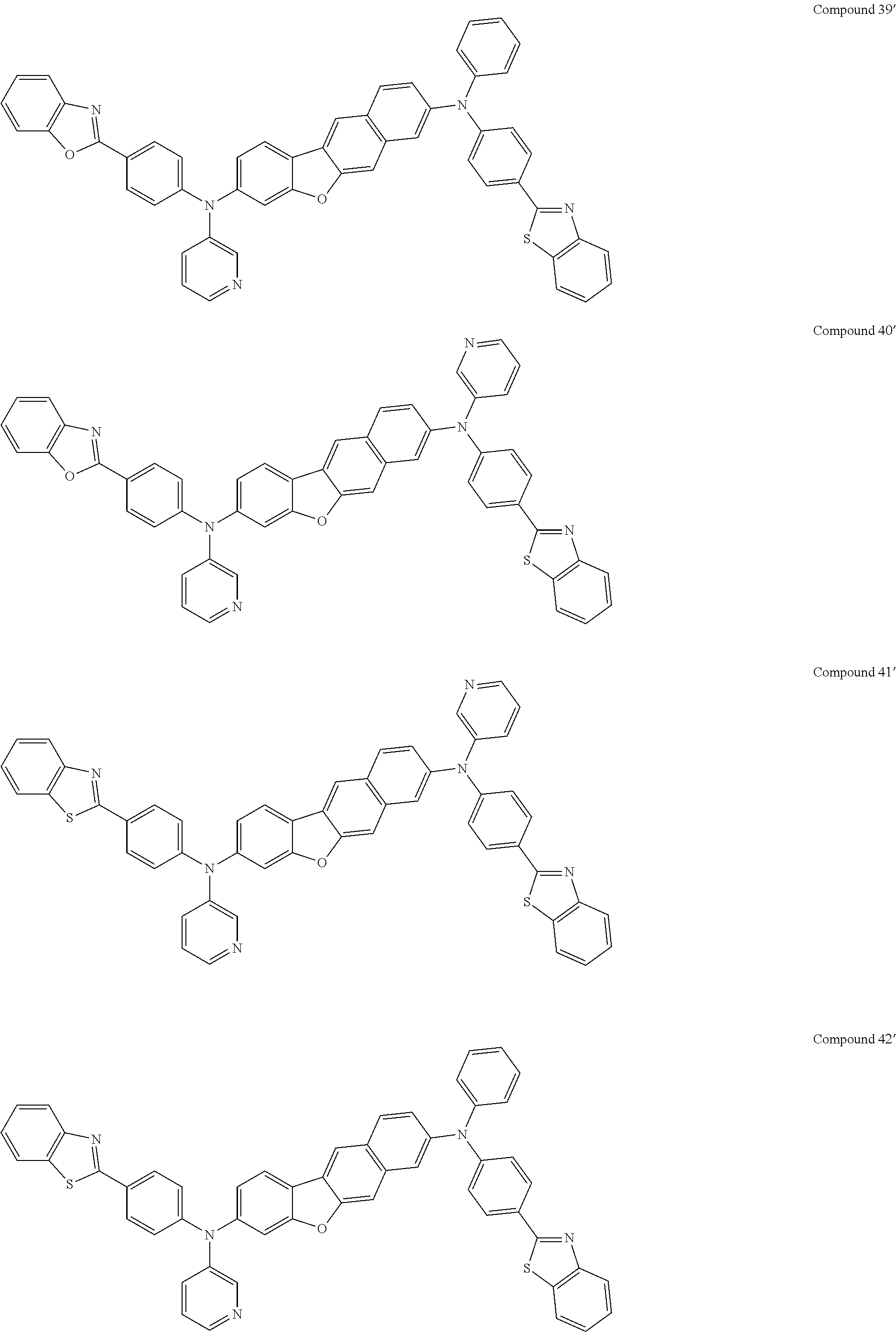

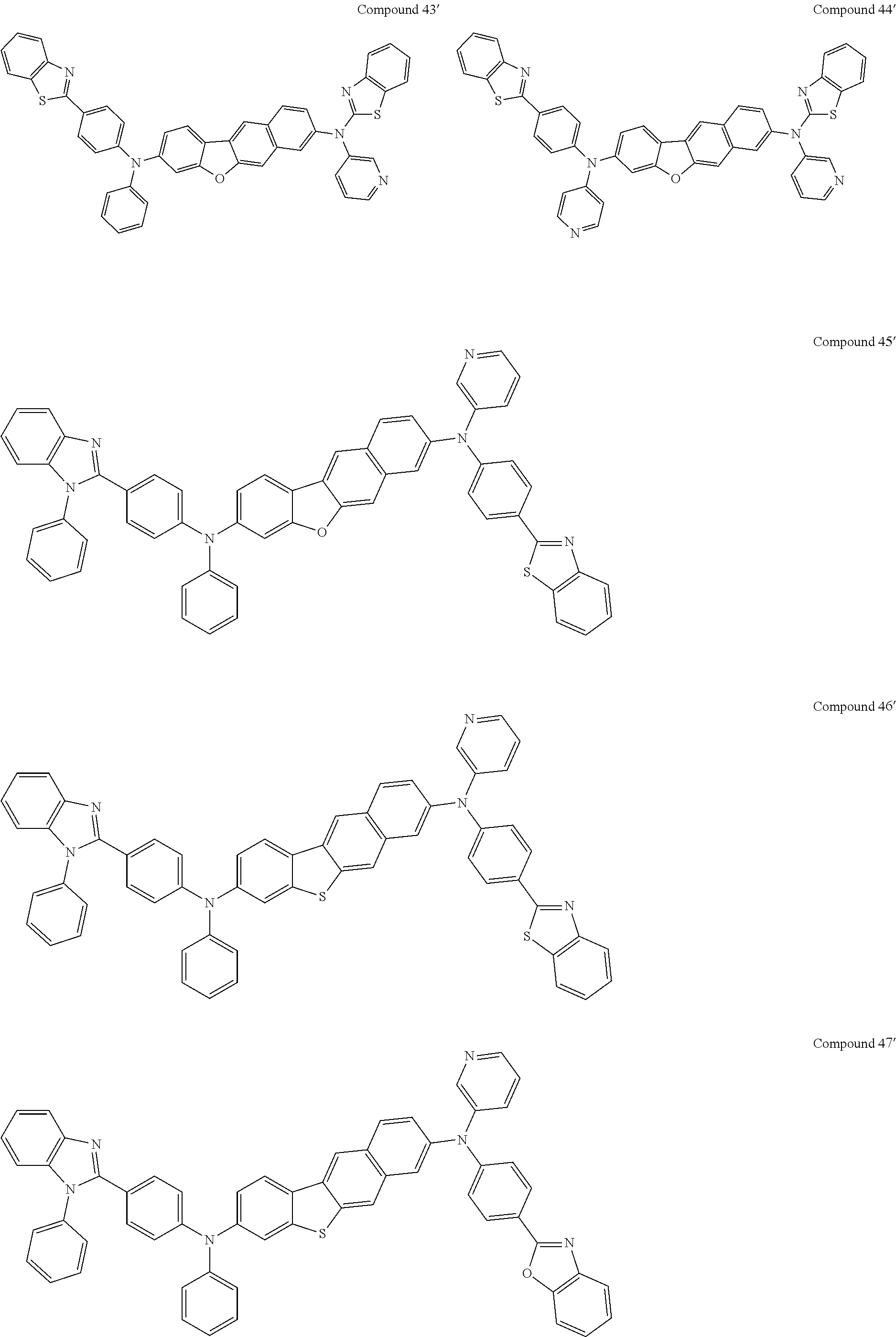

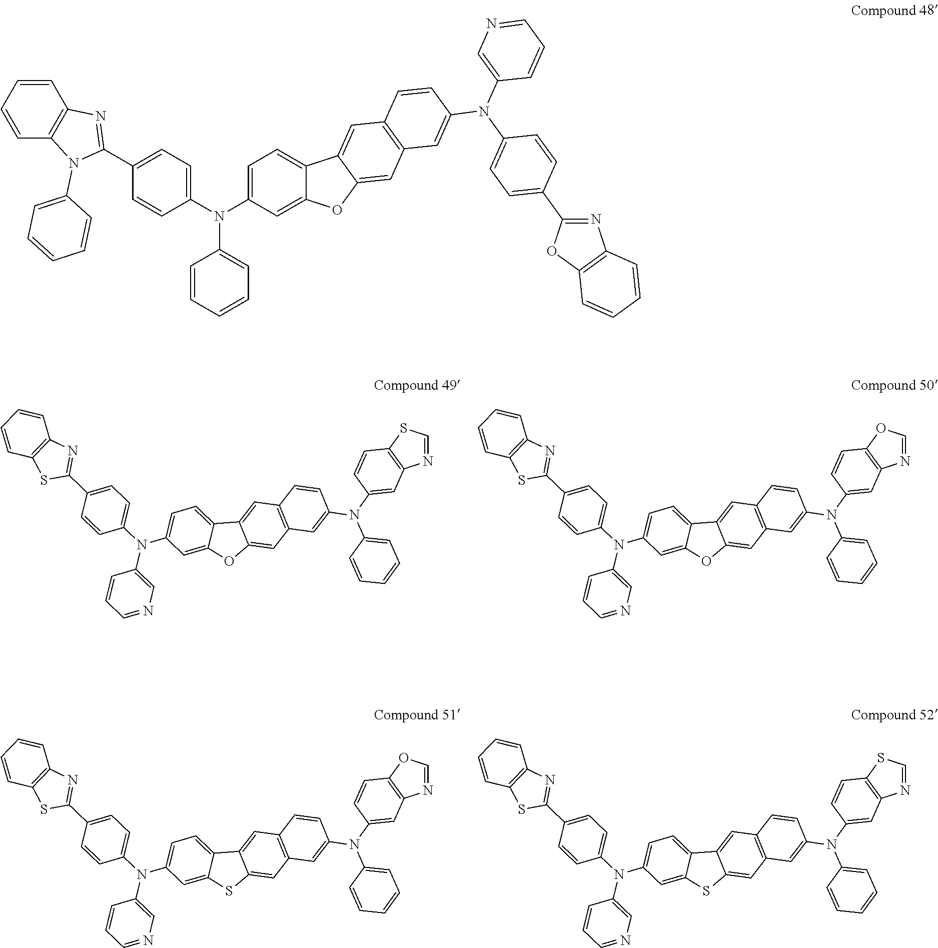

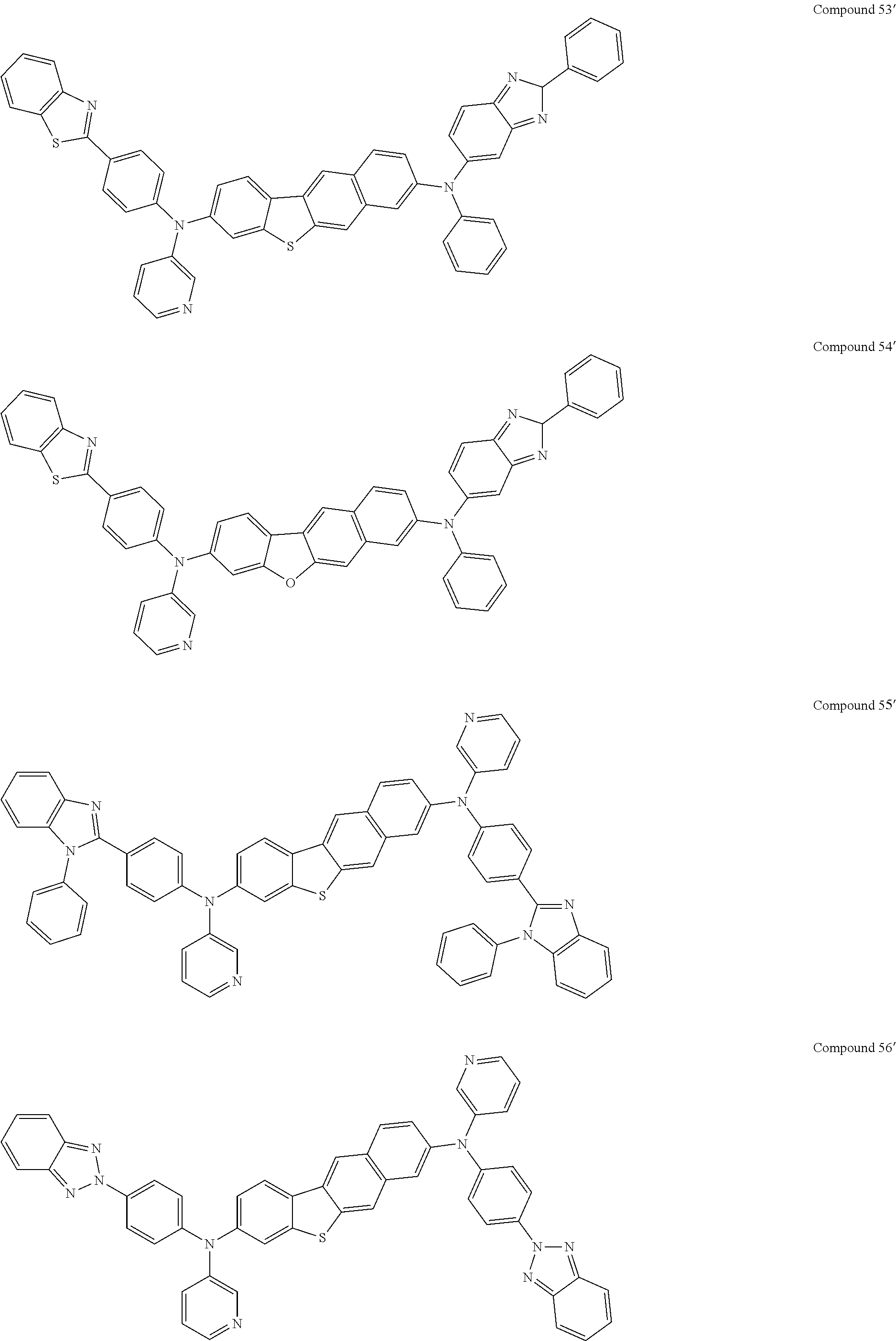

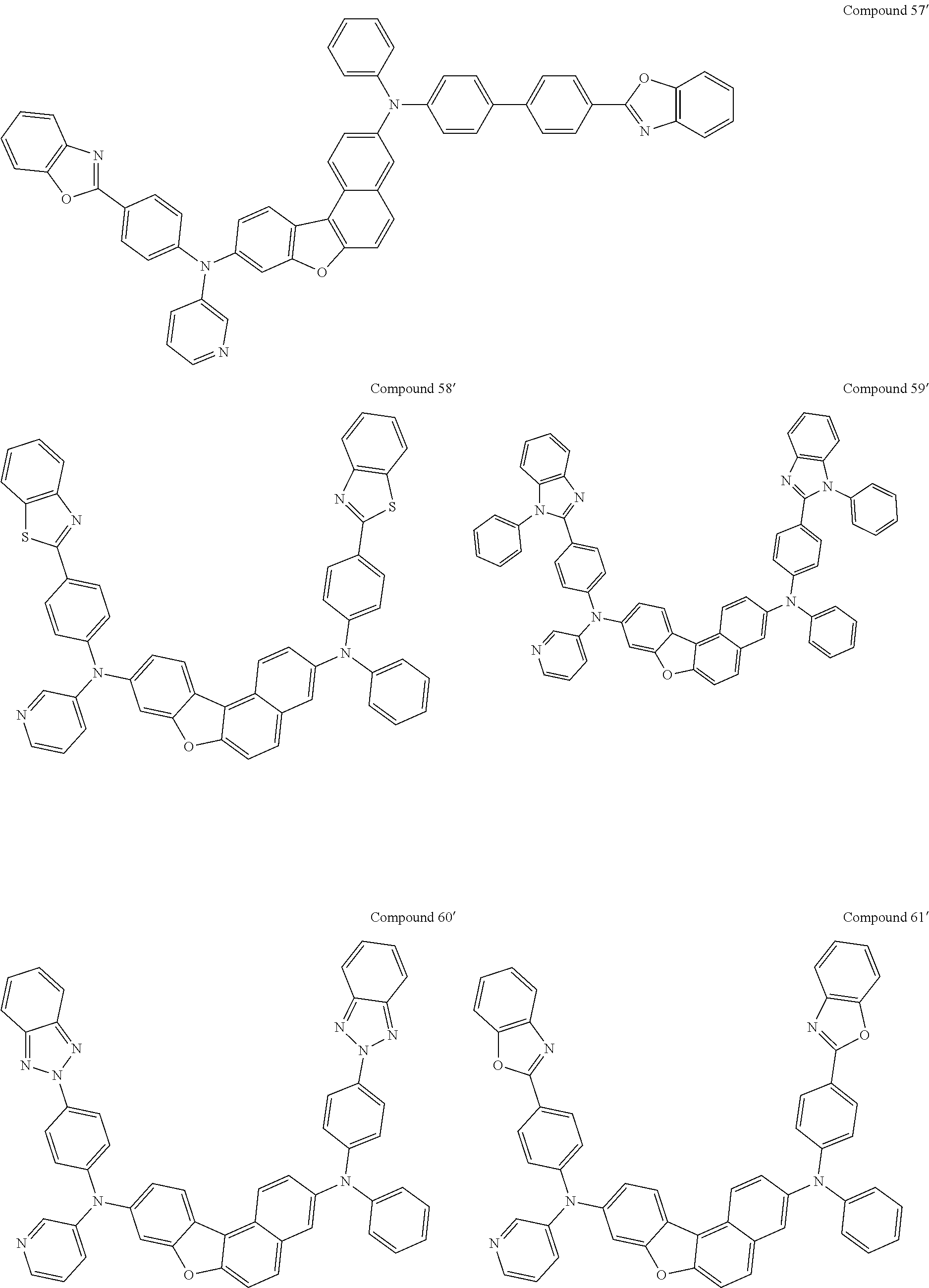

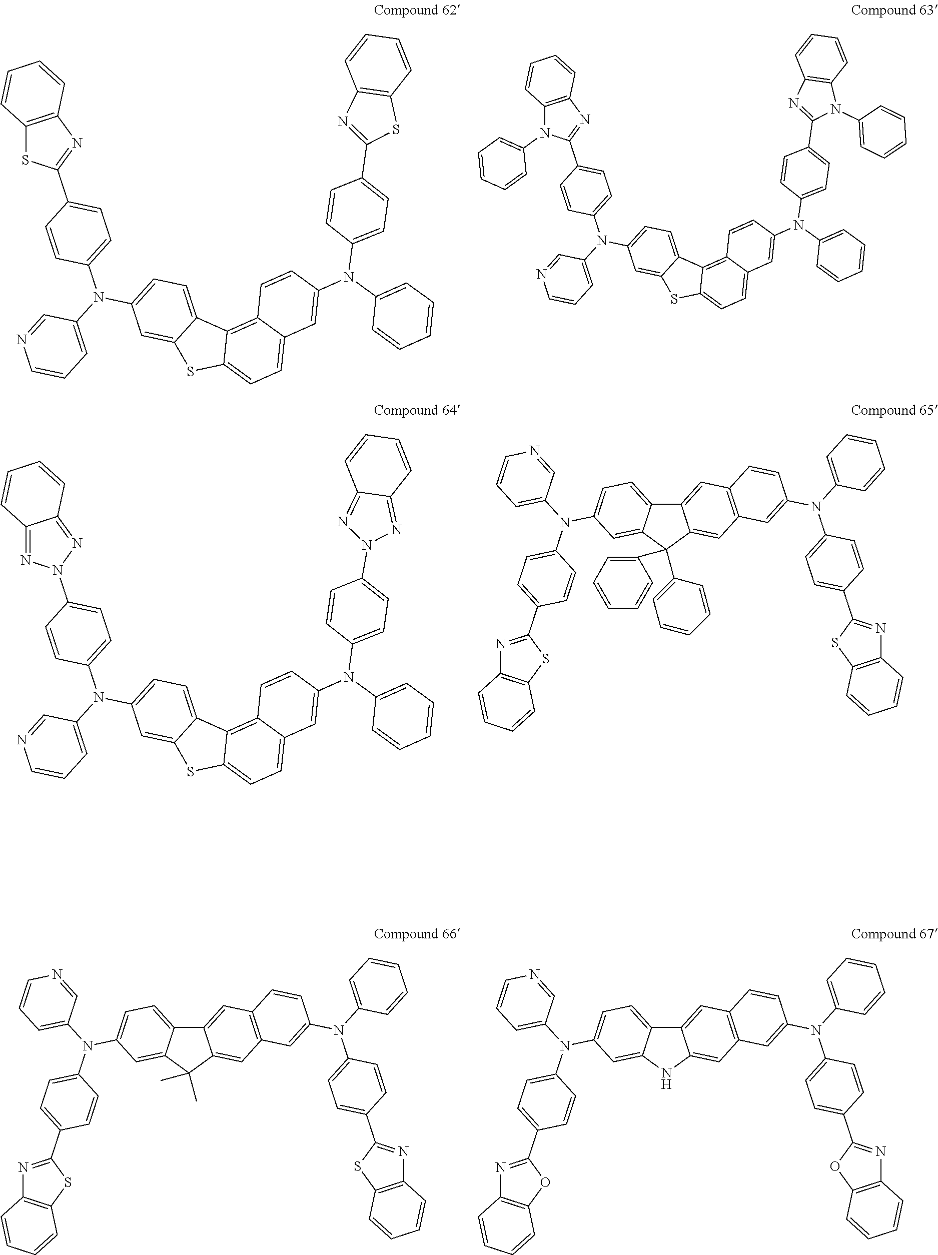

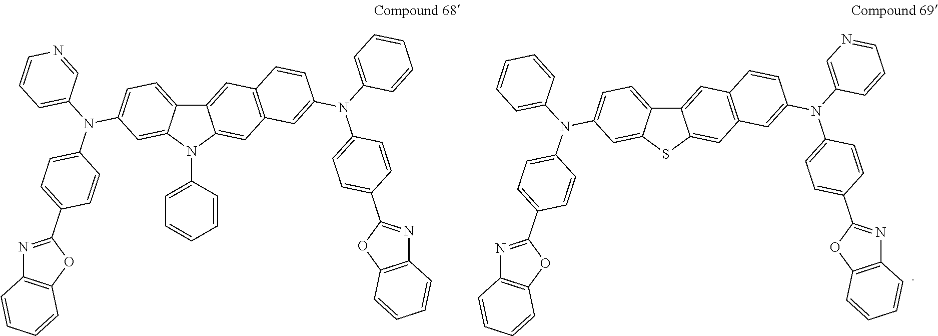

9. The diarylamine compound according to claim 1, wherein
a refractive index of the diarylamine compound at a wavelength of 460 nm is in a range of 2.08 to 2.25;
a refractive index of the aromatic amine compound at a wavelength of 530 nm is in a range of 1.92 to 2.16; and
a refractive index of the aromatic amine compound at a wavelength of 620 nm is in a range of 1.88 to 2.07.

10. The diarylamine compound according to claim 1, wherein a light absorption coefficient of the diarylamine compound at a wavelength of 400 nm is 0.84 or more, and the light absorption coefficient at a wavelength of 450 nm or more is 0.

11. The diarylamine compound according to claim 1, wherein a glass transition temperature of the diarylamine compound is 127° C. or above.

12. Use of the diarylamine compound according to claim 1 as a light extraction material.

13. A light extraction material, comprising the diarylamine compound according to claim 1.

14. An electroluminescent device, comprising the diarylamine compound according to claim 1.

15. The electroluminescent device according to claim 14, comprising: an anode, a hole injection layer, a hole transport layer, an electron block layer, an emitting layer, a hole block layer, an electron transport layer, an electron injection layer, a cathode and a light extraction layer.

16. The electroluminescent device according to claim 15, wherein
a) a material of the hole injection layer comprises transition metal oxides;
the transition metal oxides comprise any one or more of molybdenum oxides, titanium oxides, vanadium oxides, rhenium oxides, ruthenium oxides, chromium oxides, zirconium oxides, hafnium oxides, tantalum oxides, silver oxides, tungsten oxides, and manganese oxides; or
b) the material of the hole injection layer comprises a p-type dopant and a hole transport material;
the p-type dopant comprises any one or more of 2,3,6,7,10,11-hexocyano-1,4,5,8,9,12-hexaazabenzophenanthrene; 2,3,5,6-tetrafluoro-7,7′,8,8′-tetracyano-p-benzoquinone; and 1,2,3-tris[(cyano)(4-cyano-2,3,5,6-tetrafluorophenyl)methylene]cyclopropane; and
the hole transport material comprises any one or more of an arylamine hole transport material, a dimethylfluorene hole transport material, and a carbazole hole transport material.

17. The electroluminescent device according to claim 15, wherein a material of the hole transport layer comprises any one or more of an arylamine hole transport material, a dimethylfluorene hole transport material, and a carbazole hole transport material; and
a material of the electron block layer comprises any one or more of an arylamine electron block material, a dimethylfluorene electron block material, and a carbazole electron block material.

18. The electroluminescent device according to claim 15, wherein the electroluminescent device is a blue electroluminescent device, a green electroluminescent device, or a red electroluminescent device, a material of the emitting layer of the blue electroluminescent device comprises a blue luminescent material, a material of the emitting layer of the green electroluminescent device comprises a green luminescent material, and a material of the emitting layer of the red electroluminescent device comprises a red luminescent material;
the blue luminescent material comprises any one or more of a pyrene derivative-based blue luminescent material, an anthracene derivative-based blue luminescent material, a fluorene derivative-based blue luminescent material, a perylene derivative-based blue luminescent material, a styrylamine derivative-based blue luminescent material, and a metal complex-based blue luminescent material;
the green luminescent material comprises any one or more of a coumarin dye, a quinacridone derivative green luminescent material, a polycyclic aromatic hydrocarbon green luminescent material, a diamine anthracene derivative green luminescent material, a carbazole derivative green luminescent material, and a metal complex green luminescent material; and
the red luminescent material comprises any one or more of a DCM-based red luminescent material and a metal complex-based red luminescent material.

19. The electroluminescent device according to claim 15, wherein the material of the hole block layer comprises any one or more of hole block materials based on benzimidazole and its derivatives, hole block materials based on imidazopyridine and its derivatives, hole block materials based on benzimidazophenanthridine derivatives, hole block materials based on pyrimidine and its derivatives, hole block materials based on triazine derivatives, hole block materials based on pyridine and its derivatives, hole block materials based on pyrazine and its derivatives, hole block materials based on quinoxaline and its derivatives, hole block materials based on diazole and its derivatives, hole block materials based on quinoline and its derivatives, hole block materials based on isoquinoline derivatives, hole block materials based on phenanthroline derivatives, hole block materials based on diazaphosphole, hole block materials based on phosphine oxide, hole block materials based on aromatic ketone, lactams, and hole block materials based on boranes;
the material of the electron transport layer comprises any one or more of electron transport materials based on benzimidazole and its derivatives, electron transport materials based on imidazopyridine and its derivatives, electron transport materials based on benzimidazophenanthridine derivatives, electron transport materials based on pyrimidine and its derivatives, electron transport materials based on triazine derivatives, electron transport materials based on pyridine and its derivatives, electron transport materials based on pyrazine and its derivatives, electron transport materials based on quinoxaline and its derivatives, electron transport materials based on diazole and its derivatives, electron transport materials based on quinoline and its derivatives, electron transport materials based on isoquinoline derivatives, electron transport materials based on phenanthroline derivatives, electron transport materials based on diazaphosphole, electron transport materials based on phosphine oxide, electron transport materials based on aromatic ketone, lactams, and electron transport materials based on boranes; and
the material of the electron injection layer comprises any one or more of an alkali metal electron injection material and a metal electron injection material.

20. A display apparatus, comprising the electroluminescent device according to claim 14.
